(19) United States
(12) Patent Application Publication
(10) Pub. No.: US 2025/0340662 A1
(43) Pub. Date: Nov. 6, 2025

(54) BINDING PROTEIN

(71) Applicant: Akiram Therapeutics AB, Uppsala (SE)

(72) Inventors: Marika Nestor, Uppsala (SE); Helena Persson Lotsholm, Sigtuna (SE); Camilla Hofström, Upplands Väsby (SE); Maria Walle, Lund (SE); Mats Ohlin, Limhamn (SE)

(21) Appl. No.: 18/868,820

(22) PCT Filed: May 24, 2023
(86) PCT No.: PCT/EP2023/063870

§ 371 Date: Nov. 25, 2024

(30) Foreign Application Priority Data
May 25, 2022 (SE) 2250618-2

(51) Int. Cl. C07K 16/28; A61K 39/00; A61K 51/10; A61P 35/00; G01N 33/577

(52) U.S. Cl. CPC C07K 16/2884; A61K 51/1027; A61K 51/1093; A61P 35/00; G01N 33/577; A61K 2039/505; A61K 2121/00; A61K 2123/00; C07K 2317/34; C07K 2317/622; C07K 2317/92; G01N 2333/70585

(57) ABSTRACT

The current disclosure relates binding proteins that bind human CD44v6. The binding proteins may be joined to an agent to form conjugated binding, where the agent may be an imaging or therapeutic agent, such as a radioisotope. The binding proteins or conjugated biding proteins, or pharmaceutical compositions thereof, may be used in medical treatments, such as cancer therapies, or in diagnosis and medical imaging. The binding proteins may also be used for engineering cells to express a chimeric antigen receptor having a binding protein of the present disclosure as antigen binding domain.

### TECHNICAL FIELD

The present disclosure relates binding proteins that bind an epitope of human CD44v6. In some aspects, the binding proteins constitute CD44v6 binding antibodies, or fragments thereof, or conjugated binding proteins or monoclonal antibodies carrying imaging or therapeutic agents, such as antineoplastics agents. In some embodiments, the binding proteins, antibodies, conjugated biding proteins/antibodies, or pharmaceutical compositions thereof, are used in medical treatments, such as cancer therapies. In some embodiments, the binding proteins, antibodies, or conjugated biding proteins/antibodies are used in diagnosis or medical imaging. In other embodiments, the binding proteins are used for engineering cells to express a chimeric antigen receptor having a binding protein of the present disclosure as antigen binding domain.

### BACKGROUND

Cancer is one of the most prevalent deadly diseases, which despite recent advances in diagnosis and treatment still accounts for a substantial number of deaths each year. Monoclonal antibodies (mAbs), which modulate immune responses, or conjugated mAbs that carry antineoplastic agents, are providing highly effective and promising treatments for numerous cancers.

CD44 is a cell-surface glycoprotein involved in cell-cell interactions, cell proliferation, differentiation, adhesion and migration. The standard isoform CD44s comprises exons 1-5 and 16-20, while CD44 splice variants containing variable exons are designated CD44v. The alternatively spliced variant of CD44 comprising exon 6 is referred to as CD44v6. Exon v6 have been implicated in cancer, and is suggested to be related to metastatic spread, and CD44v6 have thus been identified as a potential target for cancer therapy.

A mAb against CD44v6 (BIWA-1 or VFF-18) for the treatment of squamous cell carcinomas has previously been developed (WO97/21104), as well as humanized mAb against CD44v6 (BIWA-4 or Bivatuzumab), used as a conjugated mAb in the treatment of inoperable recurrent or metastatic head and neck cancer (Postema E J, et al. Journal of Nuclear Medicine 2003, 44 (10): 1690-9).

Despite the previous developments, there is still a need for new and better binding proteins targeting CD44v6, which may be used in diagnosis and medical treatments, such as cancer therapies.

### SUMMARY

An object of the present disclosure is to provide novel and enhanced binding molecules which may be used in medical treatments, diagnosis and medical imaging. This object is obtained by a binding protein that specifically binds CD44v6 and comprises a binding domain of an antibody, the binding domain comprising a heavy chain variable domain (VH) and a light chain variable domain (VL), each comprising three complementarity determining regions (CDRs), wherein the amino acid sequences of the CDRs are selected from the group comprising: VHCDR1 as defined by SEQ ID NO: 1, VHCDR2 as defined by SEQ ID NO: 2; VHCDR3 as defined by SEQ ID NO: 3; VLCDR1 as defined by SEQ ID NO: 4; VLCDR2 as defined by X₁AS, where X₁ may be T, A, or S; VLCDR3 as defined by SEQ ID NO: 6; and CDR sequences having 95% or more, such as 96%, 97%, 98%, 99% or more, identity thereto, wherein said binding protein recognises an epitope of CD44v6 as defined by SEQ ID NO: (7). In some embodiments, the amino acid sequences of the CDRs of the binding protein is

VHCDR1 as defined by SEQ ID NO: 11, VHCDR2 as defined by SEQ ID NO: 19, VHCDR3 as defined by SEQ ID NO: 3, VLCDR1 as defined by SEQ ID NO: 26, VLCDR2 as defined by sequence TAS, and VLCDR3 as defined by SEQ ID NO: 6.

In some embodiments, the VHCDR1, VHCDR2 and VLCDR2 of the binding protein are present next to specific framework amino acids, wherein the CDR and framework amino acid (faa) sequences are selected from the group comprising: VHCDR1 and faa defined by SEQ ID NO: 8; VHCDR2 and faa as defined by SEQ ID NO: 9; VLCDR2 and faa as defined by SEQ ID NO: 10; and CDR sequences having 95% or more, such as 96%, 97%, 98%, 99% or more, identity thereto.

In some aspects, the binding protein is a monoclonal antibody, or an antigen-binding fragment selected from the group consisting of Fv fragments, Fab-like fragments and domain antibodies. In some embodiments, the Fv fragment is an scFv fragment. In some embodiments, the Fab-like fragment is a Fab or F(ab′)2 fragment. In some embodiments, the binding molecule is a monoclonal antibody of the IgG1 isotype, such as an IgG1 LALA antibody or IgG1 IAHA antibody. Typically, the binding protein is human or of human origin.

In some aspects, a conjugated binding protein is provided. The conjugated binding protein comprising: (i) at least one binding protein; and (ii) at least one agent.

The agent may be a therapeutic agent, such as cytotoxic agent. The cytotoxic agent is selected from radioisotopes, cytostatic drugs, toxins, and chemotherapeutic agents. In some embodiments, the therapeutic agent is a ¹⁷⁷Lu radioisotope.

The agent may be a detectable agent, such as a radioisotope, an enzyme, a fluorescent molecule, a dye, digoxigenin, or biotin. In some embodiments, the detectable agent is a ¹¹¹In radioisotope.

In some aspects, the agent is joined to the binding protein via a linker is in the form of a chelator. The chelator may be selected from the group consisting of derivatives of 1,4,7,10-tetraazacyclododecane-1,4,7,10, tetraacetic acid (DOTA), derivatives of deferoxamine (DFO), derivatives of diethylenetriaminepentaacetic avid (DTPA), derivatives of S-2-(4-Isothiocyanatobenzyl)-1,4,7-triazacyclononane-1,4,7-triacetic acid (NOTA), derivatives of (tBu) 4 (1-(1-carboxy-3-carbotertbutoxypropyl)-4,7,10-(carbotertbut-oxymethyl)-1,4,7,10-tetraazacyclododecane) (DOTAGA), derivatives of 1,4,8,11-tetraazacyclodocedan-1,4,8,11-tetraacetic acid (TETA), derivatives of 1,4,7-triazacyclononane, 1-glutaric acid-4,7-acetic acid (NODAGA), derivatives of 1,4,7-Triazacyclononane-1,4,7-triacetic acid (NOTA).

In some aspects, an engineered cell is provided. The cell may be engineered to express a chimeric antigen receptor (CAR), wherein the CAR comprises an antigen-binding domain, a transmembrane domain connected to the antigen binding domain by a hinge region, and an intracellular domain optionally connected to one or more co-stimulatory domains, wherein the antigen binding domain comprises an scFv fragment of a binding protein of the present disclosure.

According to some aspects, a pharmaceutical composition is provided. The pharmaceutical composition comprises a binding protein, a conjugated binding protein or an engineered cell as described above, and a pharmaceutically acceptable carrier or excipient.

The binding protein, conjugated binding protein, engineered cell, or pharmaceutical composition as described above may be for use in therapy. In some embodiments, the therapy is cancer therapy, including advanced thyroid cancer, head and neck cancer, pancreatic cancer, squamous cell carcinoma, Hodgkin lymphoma, colorectal cancer, liver cancer, cervical cancer, gastric cancer, ovarian cancer, lung cancer, bladder cancer, acute myeloid leukemia, chronic lymphocytic leukemia, multiple myeloma, breast cancer, hepatocellular cancer, and esophageal cancer, and metastatic cancers of the brain. In some embodiments, the cancer is advanced thyroid cancer.

According to some aspects, the disclosure proposes an in vitro (including in cellulo and ex vivo) method for detecting expression of the CD44 variant CD44v6, the method comprising: (i) contacting a binding protein, or a conjugated binding protein, as described above to a biological sample, such as a tissue sample or liquid, obtained from a subject, such that the binding protein or conjugated binding protein binds to an epitope of CD44v6 as defined by SEQ ID NO: 7 if present in the biological sample, (ii) washing the biological sample to remove unbound binding proteins or conjugated binding proteins, and (iii) detecting any binding proteins or conjugated binding proteins that have bound the epitope in the biological sample.

According to some aspects, the disclosure proposes an in vivo method for detecting expression of the CD44 variant CD44v6, the method comprising: (i) administering a conjugated binding protein to a subject, wherein the conjugated binding protein binds an epitope of CD44v6 as defined by SEQ ID NO: 7, and (ii) detecting that the conjugated binding protein has bound a cell expressing the epitope.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

The above, as well as additional objects, features and advantages of the present inventive concept, will be better understood through the following illustrative and non-limiting detailed description of different embodiments of the present inventive concept, with reference to the appended drawings, wherein:

FIG. 1 illustrates the generic concept of an antibody conjugated to an agent as payload.

FIG. 2 is a schematic representation of GST-CD44 (v3-v10) fusion protein, where the arrow marks a thrombin cleavage site.

FIG. 3 illustrates the basic mechanism of molecular radiotherapy using a monoclonal antibody liked to radioisotopes for targeting cancer cells.

FIG. 4 is an illustration of a CD44v6-targeting radiopharmaceutical for molecular radiotherapy of advanced thyroid cancer.

FIG. 5 illustrates the outcome of one treatment of a radiopharmaceutical of the present disclosure in mice with ATC, FIG. 5a showing tumor volume in the mice, and FIG. 5b the survival rate.

FIG. 6 is an illustration of the epitope of human CD44v6 bound by the present binding proteins, as compared to the previous BIWA-4 antibody.

FIG. 7 illustrates binding of ¹²⁵I-U-MN114-19 and ¹²⁵I-BIWA-4 to BHT-101 cells at 1 and 3 nM (U-MN114-19) and 1, 3 and 10 nM (BIWA-4).

FIG. 8 illustrates a direct comparison of a biodistribution with ¹²⁵I-U-MN114-19 with ¹²⁵I-BIWA-4. FIG. 8a shows tumor and blood curves of ¹²⁵I-U-MN114-19 and ¹²⁵I-BIWA-4 as % ID/g as IgG4 in ACT-1 xenografts. FIG. 8b shows Left: Tumor-to-organ ratios from biodistribution of ¹²⁵I-U-MN114-19 (IgG4) (N=12), Right: ¹²⁵I-BIWA-4 (IgG4) (N=11), Error bars represent SD.

FIG. 9 shows biodistributions (top) of ¹²⁵I-U-MN114-19 (IgG4) and ¹⁷⁷Lu-U-MN114-19 (IgG4), and tumor-to-organ ratios from said biodistributions (bottom).

FIG. 10 shows LigandTracer comparisons of different antibodies. FIG. 10a shows LigandTracer comparison of ¹²⁵I-labeled MN114-antibodies and ¹²⁵I-BIWA-4 on BHT-101 cells normalized to CPS at end of association. FIG. 10b shows comparison of U-MN114-19 (dark grey), AL-MN114-465 (black) and BIWA-4 (light grey). Two concentrations, 1 nM and 3 nM, for MN114-clones and three concentrations, 1 nM, 3 nM and 10 nM, for BIWA-4, were run for approximately 90 min each before starting the dissociation.

FIG. 11 illustrates the specificity in the presence of an excess 50-100-fold molar excess of non-radiolabeled antibody (50-fold of BIWA-4 for radiolabeled BIWA-4, 100-fold of U-MN114-19 for radiolabeled U-MN114-19, 100-fold of U-MN114-19 for radiolabeled AL-MN114-465).

FIG. 12 illustrates LigandTracer evaluation of antibody-retention in competition with 3-fold molar excess of non-radiolabeled antibody of ¹²⁵I-U-MN114-19 (10 nM) and ¹²⁵I-AL-MN114-444 (10 nM) on BHT-101 cells.

FIG. 13 shows biodistributions, Top: Biodistribution of ¹²⁵I-U-MN114-19 (IgG1 LALA). Bottom: Biodistribution of ¹²⁵I-U-MN114-19 (IgG1 LALA/IAHA). ACT-1 xenograft model, error bars represent SD, N=13 (LALA) and N=25 (LALA/IAHA).

FIG. 14 illustrates tumor retention, Left: Tumor retention from 24 h p.i. to 168 h p.i. of ¹²⁵I-U-MN114-19 (IgG1 LALA/IAHA) and ¹²⁵I-AL-MN114 variants (IgG1 LALA/IAHA). Right: Tumor retention from 24 h p.i. to 168 h p.i. of ¹⁷⁷Lu-U-MN114-19 (IgG1 LALA/IAHA) and ¹⁷⁷Lu-AL-MN114 variants (IgG1 LALA/IAHA). Error bars represent SD, n≥14, N=61.

FIG. 15 shows blood and tumor uptake presented as % ID/g of ¹⁷⁷Lu-labeled U-MN114-19 (left), AL-MN114-132 (middle) and AL-MN114-465 (right) in A431 xenografts. Error bars represent SD, n=16, N=48.

FIG. 16 illustrates tumor growth compared to the isotope control. Left column, top-to-bottom: ACT-1 tumor volume measurements, survival and animal weights. Right column, top-to-bottom: BHT-101 tumor volume measurements, survival and animal weights. Error bars represent SD, N=10 (5+5) for ACT-1 studies, N=8 (4+4) for BHT-101 studies.

FIG. 17 illustrates comparison of tumor uptake of ¹²⁵I-U-MN114-19 and ¹²⁵I-BIWA4 in ACT-1 xenografts.

FIG. 18 illustrates tumor growth following treatment with 10 MBq of ¹⁷⁷Lu-AL-MN114-465 or ¹⁷⁷Lu-BIWA4 in BHT-101 xenografts.

FIG. 19 illustrates time to complete response, where FIG. 19 (top) shows time to complete response of ¹⁷⁷Lu-AL-MN114-465 or ¹⁷⁷Lu-BIWA4 in BHT-101 xenografts, and FIG. 19 (bottom) shows time to partial response of ¹⁷⁷Lu-AL-MN114-465 or ¹⁷⁷Lu-BIWA4 in BHT-101 xenografts FIG. 20 illustrates the size/growth ratios of the tumors, where FIG. 20 (top) shows growth ratios of 3D multicellular tumor spheroids of BHT-101 cells treated with 60 kBq of either ¹⁷⁷Lu-AL-MN114-465, BIWA4 or an isotope control (ISO-c) antibody, and FIG. 20 (bottom) shows a one-way ANOVA of the size ratios at Day 10 post treatment.

The figures are not necessarily to scale, and generally only show parts that are necessary in order to elucidate the inventive concept, wherein other parts may be omitted or merely suggested.

### DETAILED DESCRIPTION

The present disclosure relates to new binding proteins, such as monoclonal antibodies or antigen binding fragments thereof, which selectively bind CD44v6, and to conjugated binding proteins carrying therapeutic agents, such as antibody drug conjugates (ADCs). The binding proteins, antibodies or conjugated antibodies may be used in medical treatments, such as cancer therapies, or in imaging applications, for in vitro and in vivo diagnosis. The binding proteins may also be used for engineering cells to express a chimeric antigen receptor having a binding protein of the present disclosure as antigen binding domain.

The aim of the present disclosure is to provide new and enhanced binding proteins specific for CD44v6, which may be used in therapy, diagnosis, medical imaging and cell engineering.

Aspects of the present disclosure will be described more fully hereinafter with reference to the accompanying drawings. The binding proteins, conjugated binding proteins/ADCs and methods disclosed herein can, however, be realized in many different forms and should not be construed as being limited to the aspects set forth herein. Like numbers in the drawings refer to like elements throughout.

The terminology used herein is for the purpose of describing particular aspects of the disclosure only, and is not intended to limit the disclosure. As used herein, the singular forms “a”, “an” and “the” are intended to include the plural forms as well, unless the context clearly indicates otherwise.

In some embodiments a non-limiting term “binding protein” is used. The term “binding protein” is used herein to denote a binding protein comprising a binding domain of an antibody (that is to say, a binding domain obtained or derived from an antibody, or based on a binding domain of an antibody). Thus, the binding protein is an antibody-based, or antibody-like, molecule comprising the binding site of, or a binding site derived from, an antibody. It is thus an immunological binding agent.

In some embodiments non-limiting terms “antibody” or “antigen binding fragment thereof” is used. The term “antibody” is used herein in its broadest sense, including both monoclonal and polyclonal antibodies. As is well known, antibodies are immunoglobulin molecules capable of specific binding to a target (an antigen), such as a protein, carbohydrate, polynucleotide, lipid, polypeptide or other, through at least one antigen recognition site located in the variable region of the immunoglobulin molecule. As used herein, the term “antibody” or “an antigen binding fragment thereof” encompasses not only full-length or intact polyclonal or monoclonal antibodies, but also antigen-binding fragments thereof, such as Fab, Fab′, F(ab′)2, Fab3, Fv and variants thereof, fusion proteins comprising one or more antibody portions, humanized antibodies, chimeric antibodies, minibodies, diabodies, triabodies, tetrabodies, linear antibodies, single chain antibodies, multispecific antibodies (e.g. bispecific antibodies) and any other modified configuration of the immunoglobulin molecule that comprises an antigen recognition site of the required specificity, including glycosylation variants of antibodies, amino acid sequence variants of antibodies and covalently modified antibodies.

As is known to the skilled person, antibodies are proteins which comprise four polypeptide chains: two heavy chains and two light chains. Typically, the heavy chains are identical to each other and the light chains are identical to each other. The light chains are shorter (and thus lighter) than the heavy chains. The heavy chains comprise four or five domains: at the N-terminus a variable (VH) domain is located, followed by three or four constant domains (from N-terminus to C-terminus CH1, CH2, CH3 and, where present, CH4, respectively). The light chains comprise two domains: at the N-terminus a variable (VL) domain is located and at the C-terminus a constant (CL) domain is located. In the heavy chain an unstructured hinge region is located between the CH1 and CH2 domains. The two heavy chains of an antibody are joined by disulphide bonds formed between cysteine residues present in the hinge region, and each heavy chain is joined to one light chain by a disulphide bond between cysteine residues present in the CH1 and CL domains, respectively. In mammals two types of light chain are produced, known as lambda (λ) and kappa (κ). For kappa light chains, the variable and constant domains can be referred to as V_{K} and C_{K} domains, respectively. Whether a light chain is a λ or κ light chain is determined by its constant region: the constant regions of λ and κ light chains differ, but are the same in all light chains of the same type in any given species. Depending on the amino acid sequence of the constant domain of its heavy chains, antibodies are assigned to different classes. There are six major classes of antibodies: IgA, IgD, IgE, IgG, IgM and IgY, and several of these may be further divided into subclasses, e.g., IgG1, IgG2, IgG3, IgG4, IgA1 and IgA2. The term “full-length antibody” as used herein, refers to an antibody of any class, such as IgD, IgE, IgG, IgA, IgM or IgY (or any sub-class thereof). The term “antigen binding fragment” refers to a portion or region of an antibody molecule, or a derivative thereof, that retains all or a significant part of the antigen binding of the corresponding full-length antibody. In some embodiments, the heavy chain of the antibodies may comprise VH+CH1+Hinge+CH2+CH3, and the light chain VL+CL. In preferred embodiments, the antibodies have the IgG1 LALA format, and the CH1 is defined by SEQ ID NO: 117, the CH2 is defined by SEQ ID NO: 119, the CH3 is SEQ ID NO: 120, the CL is defined by SEQ ID NO: 116 and the hinge by SEQ ID NO: 118.

As briefly listed above, examples of antigen binding fragments include, but are not limited to: (1) a Fab fragment, which is a monovalent fragment having a VL-CL chain and a VH-CH chain; (2) a Fab′ fragment, which is a Fab fragment with the heavy chain hinge region, (3) a F(ab′)2 fragment, which is a dimer of Fab′ fragments joined by the heavy chain hinge region, for example linked by a disulfide bridge at the hinge region; (4) an Fc fragment; (5) an Fv fragment, which is the minimum antibody fragment having the VL and VH domains of a single arm of an antibody; (6) a single chain Fv (scFv) fragment, which is a single polypeptide chain in which the VH and VL domains of an scFv are linked by a peptide linker; (7) an (scFv) 2, which comprises two VH domains and two VL domains, which are associated through the two VH domains via disulfide bridges and (8) domain antibodies, which can be antibody single variable domain (VH or VL) polypeptides that specifically bind antigens. Antigen binding fragments can be prepared via routine methods. For example, F(ab′)2 fragments can be produced by pepsin digestion of a full-length antibody molecule, and Fab fragments can be generated by reducing the disulfide bridges of F(ab′)2 fragments. Alternatively, fragments can be prepared via recombinant technology by expressing the heavy and light chain fragments in suitable host cells (e.g., E. coli, yeast, mammalian, plant or insect cells) and having them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. For example, a flexible linker may be incorporated between the two variable regions. Accordingly, throughout the description the generic terms “binding protein”, or “antibody” is used. These terms are used in their broadest sense and thus also incorporate all variants and fragments described above and below. In some embodiments the binding protein is a monoclonal antibody or an antigen binding fragment selected from the group consisting of Fv fragments (e.g. single chain Fv and disulphide-bonded Fv), Fab-like fragments (e.g. Fab fragments, Fab′ fragments and F(ab)2 fragments) and domain antibodies (e.g. single VH variable domains or VL variable domains).

Thus, the constant regions of the heavy chains are the same in all antibodies of any given isotype in a species, but differ between isotypes. The specificity of an antibody is determined by the sequence of its variable region. The sequence of variable regions varies between antibodies of the same type in any individual. In particular, both the light and heavy chains of an antibody comprise three hypervariable complementarity-determining regions (CDRs). In a pair of a light chain and a heavy chain, the CDRs of the two chains form the antigen-binding site. The CDR sequences determine the specificity of an antibody. A pair of a light chain variable region and a heavy chain variable region, comprising an (antigen) binding site, is known as an (antigen) binding domain. The three CDRs of a heavy chain are known as VHCDR1, VHCDR2 and VHCDR3, from N-terminus to C-terminus, and the three CDRs of a light chain are known as VLCDR1, VLCDR2 and VLCDR3, from N-terminus to C-terminus.

In an antibody, as described above, the CDR sequences are located in the variable domains of the heavy and light chains. The CDR sequences sit within a polypeptide framework, which positions the CDRs appropriately for antigen binding. Thus, the remainder of the variable domains (i.e. the parts of the variable domain sequences which do not form a part of any one of the CDRs) constitute framework regions. The N-terminus of a mature variable domain forms framework region 1 (FR1); the polypeptide sequence between CDR1 and CDR2 forms FR2; the polypeptide sequence between CDR2 and CDR3 forms FR3; and the polypeptide sequence linking CDR3 to the constant domain forms FR4. In a binding protein of the invention the variable region framework regions may have any appropriate amino acid sequence such that the binding protein binds to CD44v6 via its CDRs.

If the binding protein is an antibody, the antibody may be of any isotype and sub-type. Thus, it may be an IgA, IgD, IgE, IgG, or IgM antibody. The heavy-chain constant domains that correspond to the different isotypes of immunoglobulins are termed α, δ, ε, γ and μ, respectively. The subunit structures and three-dimensional configurations of different isotypes of immunoglobulins are well known. Preferably the antibody is an IgG antibody. As noted above, there are four sub-types of IgG antibody: IgG1, IgG2, IgG3 and IgG4. The IgG anti-CD44v6 antibody of the invention may be of any IgG sub-type, i.e. it may be an IgG1, IgG2, IgG3 or IgG4 antibody. In a preferred embodiment, the antibody is an IgG1 or IgG4 antibody, such as an IgG1 LALA or and IgG1 IAHA antibody. In IgG1 LALA the leucines (L) have been substituted for alanines (A) in amino acid positions 234 and 235 in the Fc region. The LALA mutation removes Fc mediated binding to the Fcγ receptor of immune cells which diminishes effector functions. Removing the binding thereby evades immune reactions, i.e. decreases immunogenicity mediated by Fc-effector functions such as ADCC and CDC and the risk of the biopharmaceutical causing unwanted off target and on target side effects. In an IAHA mutation isoleucine (I) has been substituted for alanine (A) and histidine (H) for alanine (A) at amino acid positions 253 and 310 in the Fc region of the antibody. Thus, in this embodiment, the binding proteins may be antibodies designed as a “silent Fc” no Fc-gamma receptor interaction antibody through the LALA mutation to ablate ADCC/CDC activity. Moreover, the antibodies have been characterized for low risk of immunogenicity as assessed in in silico T-cell epitope prediction analysis The IAHA double mutation in the Fc region diminishes the interaction with FcRn (FcRn binding), which in turn deceases its circulation time in the blood (DOI: 10.1080/19420862.2016.1156285).

As detailed above, antibody light chains belong to either the kappa (κ) and lambda (λ) types. The binding protein of the present invention may contain κ or λ light chains. In a particular embodiment the binding protein of the present invention comprises a κ light chain.

Alternatively, the binding protein may be a binding fragment of an antibody (i.e. an antibody fragment), that is a fragment which retains the ability of the antibody to bind specifically to CD44v6. Such fragments are well-known, and examples include Fab′, Fab, F(ab′)₂, Fv, Fd, or dAb fragments, which may be prepared according to techniques well known in the art.

A Fab fragment consists of the antigen binding domain of an antibody, i.e. an individual antibody may be seen to contain two Fab fragments, each consisting of a light chain and its conjoined N-terminal section of a heavy chain. Thus, a Fab fragment contains an entire light chain and the VH and CH1 domains of the heavy chain to which it is bound. Fab fragments may be obtained by digesting an antibody with papain.

F(ab′)₂ fragments consist of the two Fab fragments of an antibody, plus the hinge regions of the heavy domains, including the disulphide bonds linking the two heavy chains together. In other words, a F(ab′)₂ fragment can be seen as two covalently joined Fab fragments. F(ab′)₂ fragments may be obtained by digesting an antibody with pepsin. Reduction of F(ab′)₂ fragments yields two Fab′ fragments, which can be seen as Fab fragments containing an additional sulfhydryl group which can be useful for conjugation of the fragment to other molecules.

Alternatively, the binding protein may be a synthetic or artificial construct, i.e. an antibody-like molecule which comprises a binding domain, but which is genetically engineered or artificially constructed. This includes chimeric or CDR-grafted antibodies, as well as single chain antibodies and other constructs, e.g. scFv, dsFv, ds-scFv, dimers, minibodies, diabodies, single domain antibodies (DABs), TandAbs dimers and heavy chain antibodies such as V_{H}H, etc. In a particular embodiment the artificial construct is a single chain variable fragment (scFv). An scFv is a fusion protein in which a single polypeptide comprises both the V_{H} and V_{L} domains of an antibody. scFv fragments generally include a peptide linker covalently joining the V_{H} and V_{L} regions, which contributes to the stability of the molecule. The linker may comprise from 1 to 20 amino acids, such as for example 1, 2, 3 or 4 amino acids, 5, 10 or 15 amino acids, or other intermediate numbers in the range 1 to 20 as convenient. The peptide linker may be formed from any generally convenient amino acid residues, such as glycine and/or serine, as familiar to the person skilled in the art. However, it is not essential that a linker be present, and the V_{L} domain may be linked to the V_{H} domain by a peptide bond. An scFv typically comprises, N-terminal to C-terminal, a V_{H} region linked to a V_{L} region by a linker sequence. The preparation of scFv molecules is well known in the art.

A binding domain of an antibody is composed of a light chain variable domain and a heavy chain variable domain (a classical bivalent antibody has two binding domains). A binding protein may thus be a native antibody or a fragment thereof, or an artificial or synthetic antibody, or an antibody construct, or derivative (e.g. a single chain antibody, as discussed further below). In summary, the binding protein of the invention comprises a binding domain of an antibody, said binding domain of an antibody comprising a light chain variable domain and a heavy chain variable domain.

As used herein, the term “capable of binding X”, wherein X is an antigen, refers to a property of an antibody or binding fragment thereof which may be tested for example by ELISA, by use of surface plasmon resonance (SPR) technology, by use of the Kinetic Exclusion Assay (KinExA®) or by bio-layer interferometry (BLI). The skilled person is aware of said methods and others.

The term “specificity”, sometimes referred to as “selectivity,” of the binding protein for a target refers to a binding protein which will bind to the target with high affinity, but typically not to other antigens. A selective or specific binding protein/antibody will not, or to a low extent, cross-react with other targets than the intended antigen. Thus, by binding “specifically” it is meant that the binding protein binds to its target (i.e. CD44v6) in a manner that can be distinguished from binding to non-target molecules, more particularly that the binding protein binds its target (CD44v6) with greater binding affinity than with which it binds other molecules. That is, the binding protein does not bind to other, non-target, molecules, or does not do so to an appreciable or significant degree, or binds with lower affinity to such other molecules than with which it binds CD44v6. A binding protein “that specifically binds” CD44v6 may alternatively be referred to as “directed against” or “that recognises” CD44v6. In other words, CD44v6 is the antigen of the binding protein of the present invention, and the binding protein is thus an “antigen binding protein” in the sense that it binds CD44v6 as its antigen.

The binding protein of the invention may be conjugated to an agent to form a conjugated binding protein. In one aspect, the agent may be a detectable agent, such as a label of some sort, and the conjugated binding protein be used in imaging. In other aspects, the conjugated binding protein may be formed by linking the binding protein to a therapeutic agent. In said case, the conjugated binding protein may be formed as, or functioning similar to, an antibody drug conjugate (ADC), and may be used in therapy.

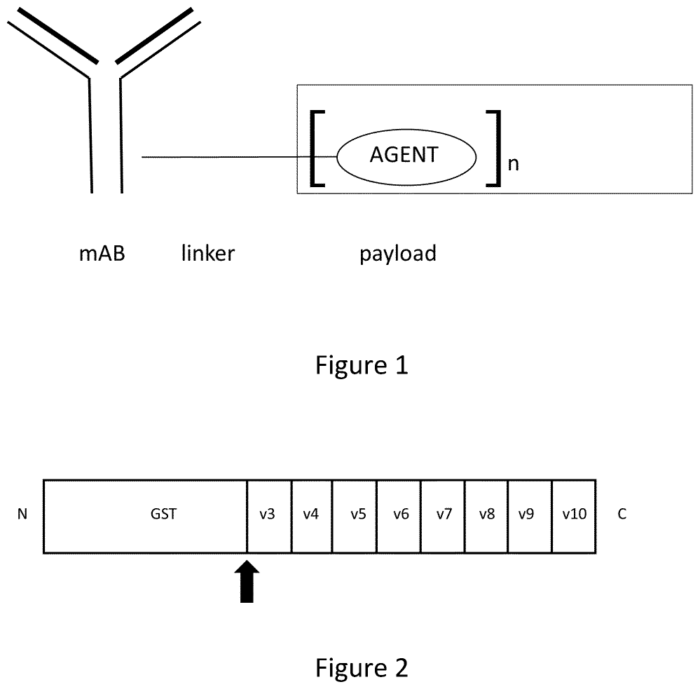

ADCs may deliver highly potent cytotoxic anticancer agents to cancer cells by joining them to monoclonal antibodies by biodegradable, stable linkers and discriminate between cancer and normal tissue. ADCs may thus combine monoclonal antibodies specific to surface antigens present on particular tumor cells with highly potent anti-cancer agents linked via a chemical linker. The ADCs typically consist of three parts: an antibody specific to the target associated antigen (selective for a tumor-associated antigen that has restricted or no expression on normal healthy cells), a payload designed to kill target cancer cells (a potent cytotoxic agent designed to induce target cell death after being internalized in the tumor cell and released), and a chemical linker to attach the payload to the antibody (a linker that is stable in circulation, but releases the cytotoxic agent in target cells), where the linkers are either cleavable or non-cleavable. ADCs are typically monoclonal antibodies covalently linked to small molecule drugs that target the specific cancer cell to reduce systemic toxicity, increase the cell-killing potential of monoclonal antibodies, and confer higher tumor selectivity, which results in higher tumor selectivity and limited systemic exposure, and thus higher drug tolerability. ADCs deliver the therapeutic agent via a linker attached to a monoclonal antibody that binds to a specific target expressed on cancer cells. After binding to the target (cancer protein or receptor), the ADC releases a cytotoxic drug into the cancer cell. The chemical “linkers” that join together the antibodies and cytotoxic drugs are highly stable to prevent cleaving (splitting) before the ADC enters the tumour. The anticancer drugs penetrate the tumour and cause cell death either by damaging the DNA of cancer cells or by preventing new cancer cells from forming and spreading. Thus, the ADCs binds proteins on the surface of cancer cells, is internalized, and release the drug while internalized, killing the cancer cell. A schematic drawing of an ADC is shown in FIG. 1, showing a mAb carrying a payload via a linker, where the agent may be present in several copies as indicated by the letter n (number of individual agents attached to the mAb).

By “therapy” as used herein is meant the treatment of any medical condition. Such treatment may be prophylactic (i.e. preventative), curative (or treatment intended to be curative), or palliative (i.e. treatment designed merely to limit, relieve or improve the symptoms of a condition). Thus, “therapy” or “treating” of a disorder, such as cancer/cancer tumors, by means of a binding protein or conjugated binding protein as used herein, are referring to preventing or ameliorating a certain disorder or medical condition, or to cure it. In the case of cancer and tumors, the treatments may shrink or abolish the present tumors, or they may halt or prevent the further spread of the tumors. An amount adequate to accomplish this is defined as a “therapeutically effective amount”. Effective amounts for a given purpose will depend on the disease or condition to be treated, its severity and the size/weight and general state of the subject. Thus, the binding proteins or conjugated binding proteins as described herein may be used in the treatment/therapy of any condition in which the target antigen is expressed/overexpressed in a subject, to ameliorate said conditions, and may be administered systemically or locally, and by any suitable method known in the art. A subject, as defined herein, refers to any mammal, e.g. a farm animal such as a cow, horse, sheep, pig or goat, a pet animal such as a rabbit, cat or dog, or a primate such as a monkey, chimpanzee, gorilla or human. Most preferably the subject is a human being.

Prophylactic treatment may include the prevention of a condition, or a delay in the development or onset of a condition. For example, the conjugated binding proteins may be used to prevent an infection, or to reduce the extent to which an infection may develop, or to prevent, delay or reduce the extent of a cancer developing, or recurring, or for example to prevent or reduce the extent of metastasis

By the term “diagnosis” or “diagnosing” as used herein is meant a process of determining if a disease or condition, in such as cancer, is present in a subject tested. A diagnosis, in the sense of diagnostic procedure, can be regarded as an attempt at classification of an individual's condition into separate and distinct categories that allow medical decisions about treatment and prognosis to be made. Subsequently, a diagnostic opinion is often described in terms of a disease or other condition. The initial task is to detect a medical indication to perform a diagnostic procedure, such as detection of any deviation from what is known to be normal. A diagnostic procedure may be performed in vitro, using the binding proteins or conjugated binding proteins herein in, for example, an enzyme-linked immunoassays (ELISA), radioimmunoassays, immunohistochemical methods or western blots, or it may be performed in vivo, where the binding proteins may carry a detectable agent, such as a label, e.g. for imaging of a tumor in vivo. The binding proteins may be combined with radioactive isotopes for imaging, such as immune scintigraphy.

The binding proteins or conjugated binding proteins may thus be used in medical imaging, which may be used in diagnosis or prognosis. With the term “prognosis” or “prognosing” as used herein is meant a prediction or estimate of the chance of recovery or survival from a disease when treated. Prognosis with cancer can depend on several factors, such as the stage of disease at diagnosis, type and subtype of cancer, the molecular profile of the tumor, and even gender. The diagnosis/prognosis may also be used to differentiate patient into different subgroups depending on nature or aggressiveness of a disorder, such as cancer. It may further be used for treatment planning, i.e. in determining a dosage regimen. In case the conjugated binding protein comprises a radionuclide, dosimetry may be used, where the diagnosis/prognosis could include determining the radiation dose by measurement, calculation, or a combination of measurement and calculation of the absorbed dose (radiation energy deposited in tissue divided by mass of the tissue) via binding/uptake/internalization of the conjugated binding protein.

The CD44 cell-surface glycoprotein plays a role in the facilitation of cell-cell and cell-matrix interactions through its affinity for hyaluronic acid. In addition, it is known to impart adhesion and is also involved in the assembly of growth factors on the cell surface, for example, EGFR and HER4. Dysfunction and/or altered expression of the protein causes various pathogenic phenotypes. The term “CD44” refers to CD44 from any species. Thus, it may be human CD44 or its equivalent or corresponding molecule in other species, most notably other mammals. Human CD44 has the UniProt accession number P16070. CD44 transcripts undergo complex alternative splicing, resulting in functionally different isoforms, where CD44s is the standard isoform and CD44v variant, as illustrated in FIG. 2. CD44 proteins are encoded by a single and highly conserved gene consisting of 20 exons, where exons 1-5, 16-18, and 20 encode the smallest, the standard, and the hematopoietic isoform CD44s. The exons that are lacking in CD44s are called CD44 exon isoform variants (referred to as CD44v1-10). Thus, ten variant exons, 6-15 (v1-v10), which are in the middle of the CD44 gene, can be alternatively spliced to yield a wide variety of CD44 variant (CD44v) isoforms, where one is the CD44 variant 6, isoform CD44v6. Additionally, 19 different splice variants have been found that are generated by alternative splicing of the CD44 mRNA, all of which are expressed at various levels in different tissues, and the roles of these variants are not fully understood. Preferably the CD44 is human CD44v6, such that the binding protein of the invention specifically binds human CD44v6.

CD44v6 is a non-internalizing cancer-associated splice variant of CD44 (hyaluronic acid receptor). High CD44v6 expression has been found in several cancers, and is associated with a poor prognosis and accelerated, aggressive disease. CD44v6 expression in normal tissue is restricted to the suprabasal stratum spinosum epithelial layers, more specifically in the keratinocytes. Thus, while CD44 is widely expressed in most vertebrate cells, the expression of CD44v6 is restricted to only a few tissues and has been considered to be associated with tumor progression and metastasis. Consequently, the low level of expression in healthy tissue, coupled with the overexpression on a variety of different cancer types, renders CD44v6 a promising target for molecular radiotherapy.

The binding protein herein may be joined, e.g. by genetic fusion, conjugated or chemically linked to an “agent”, i.e. a moiety with a certain property, to form a conjugated or fused binding protein, where the binding protein and agent may be directly joined to one another, such as when coupling an iodine (I) radioisotope to the binding protein, or may be joined via a linker (i.e. joined indirectly to one another), such as when coupling a lutetium (Lu) to the binding protein. The agent may be coupled using a chelator (a form of indirect joining), where the chelator is joined to the binding protein and chelates the agent. The agent may be radioisotope, a photoactivatable compound, a radioactive compound, an enzyme, a fluorescent dye, a biotin molecule, a toxin, a cytotoxic agent, a prodrug, a binding molecule with a different specificity, a cytokine or another immunomodulatory or cytotoxic polypeptide.

The agent may be a therapeutic agent or a detectable imaging agent. The therapeutic agents or active pharmaceutical ingredients (API) joined, conjugated or linked to the binding protein may be a cytotoxic agent that comprises or consists of one or more radioisotopes and/or one or more cytotoxic drugs. The term “radioisotope” may also be referred to as “radionuclide”, and refers to a nuclide that has excess nuclear energy, making it unstable, and prone to undergo radioactive decay. The one or more radioisotopes is or are each independently selected from the group consisting of beta-emitters, auger-emitters, conversion electron-emitters, alpha-emitters, and low photon energy-emitters, and may each independently have an emission pattern of locally absorbed energy that creates a high dose absorbance in the vicinity of the agent. The one or more radioisotopes are each independently selected from the group consisting of long-range beta-emitters, such as ⁹⁰Y, ³²P, ¹⁸⁶Re/¹⁸⁸Re; ¹⁶⁶Ho, ⁷⁶As/⁷⁷As, ¹⁵³Sm; medium range beta-emitters, such as ¹³¹I, ¹⁷⁷Lu, ⁶⁷Cu, ¹⁶¹Tb, ⁴⁷Sc; low-energy beta-emitters, such as ⁴⁵Ca, ³⁵S or ¹⁴C; conversion or auger-emitters, such as ⁵¹Cr, ⁶⁷Ga, ⁹⁹TC^{m}, ¹¹¹In, ¹²³I, ¹²⁵I, ²⁰¹Tl, and alpha-emitters, such as ²¹²Bi, ²¹²Pb, ²¹³Bi, ²²³Ac, ²²⁵Ac, ²²⁷Th, ¹⁴⁹Tb and ²¹¹At.

The binding proteins or conjugated binding proteins including a therapeutic agent of the present disclosure may be used in medicine and therapy of any condition or disorder which shows a CD44v6 expression. In some embodiments, the condition or disorder is a cancer, such as advanced thyroid cancer, head and neck cancer, pancreatic cancer, squamous cell carcinoma, Hodgkin lymphoma, colorectal cancer, liver cancer, cervical cancer, gastric cancer, ovarian cancer, lung cancer, bladder cancer, acute myeloid leukemia, chronic lymphocytic leukemia, multiple myeloma, breast cancer, hepatocellular cancer, and esophageal cancer, and metastatic cancers of the brain, including metastasizing forms of said other cancers, also when metastases have already formed. As CD44v6 has been linked to angiogenesis, the formation of new blood vessels from already existing vessels, which is essential to tumor growth and also other conditions, the present binding proteins may also act as anti-angiogenesis agents in the treatment of cancers and other angiogenesis related disorders.

In other aspects, the binding protein may be linked to an imaging agent. Molecular imaging combining imaging agents with targeting moieties in the form of a binding protein may be used to specifically image diseased sites in the body. The binding proteins may be used in molecular imaging to target imaging agents, such as radionuclides, to the cell of interest in vivo. This gives the ability to monitor disease progression and to predict response to a specific therapeutic agent, thus enabling diagnostics and response prediction for any tissue and disease where the antigen is expressed/overexpressed. The imaging/detectable agents may be radioisotopes, enzymes, fluorescent molecules, dyes, digoxigenin, and biotin, among others. The detectable agent may be detectable by an imaging technique such as SPECT, PET, MRI, optical or ultrasound imaging. When the detectable agents are radioisotopes, they may be selected from ¹¹¹In, ^{99m}Tc, ⁶⁷Ga, ⁶⁸Ga, ⁷²As, ⁸⁹Zr, ¹²³I, ¹²⁵I, ¹²⁴I, ⁴⁷Sc and ²⁰¹Tl. The conjugated binding protein may comprise a pair of detectable and cytotoxic radioisotopes, such as ⁸⁶γ/⁹⁰γ, ¹¹¹In/¹⁷⁷Lu or ¹²⁵I/²¹¹At, wherein the radioisotope is capable of simultaneously acting in a multi-modal manner as a detectable agent and also as a cytotoxic agent.

The binding proteins have been shown to bind CD44v6 with a high affinity, and may thus be used as a stand-alone cancer therapeutic or as a conjugated binding protein, as described above. The binding proteins of the present disclosure may also be used for designing chimeric antigen receptors (CARs) against CD44v6 to obtain CD44v6-targeted CAR cells, such as CAR-T cells. These CD44v6-targeted CAR T-cells can then be used in therapy to eliminate CD44v6 expressing cells, such as cancer cells. For example, a CAR T-cell is made by isolating T cells from a subject, inserting a gene for the CAR in the T-cells to create a CAR T-cell expressing a CAR protein, where the CARs are hybrids of T-cell and antibody receptors comprising 4 distinct regions; an extracellular domain which recognizes the antigen (typically an scFv fragment of an antibody) connected to a transmembrane domain by a hinge (spacer), where the transmembrane domain has a hydrophobic alpha-helix structure, and wherein the transmembrane domain is connected to an endodomain (intracellular domain), which undergoes conformational changes following antigen recognition, which triggers downstream signalling pathways to induce immune responses. The endodomain may also comprise one or more co-stimulatory domains to enhance the anti-tumor activity. Thus, the present disclosure provides cell engineered to express a chimeric antigen receptor (CAR), wherein the CAR comprises an antigen-binding domain, a transmembrane domain connected to the antigen binding domain by a hinge region, and an intracellular domain optionally connected to one or more co-stimulatory domains, wherein the antigen binding domain comprises an scFv fragment of the binding protein. The cell may be a human cell, an immune effector cell, such as a T cell, an NK cell or a macrophage.

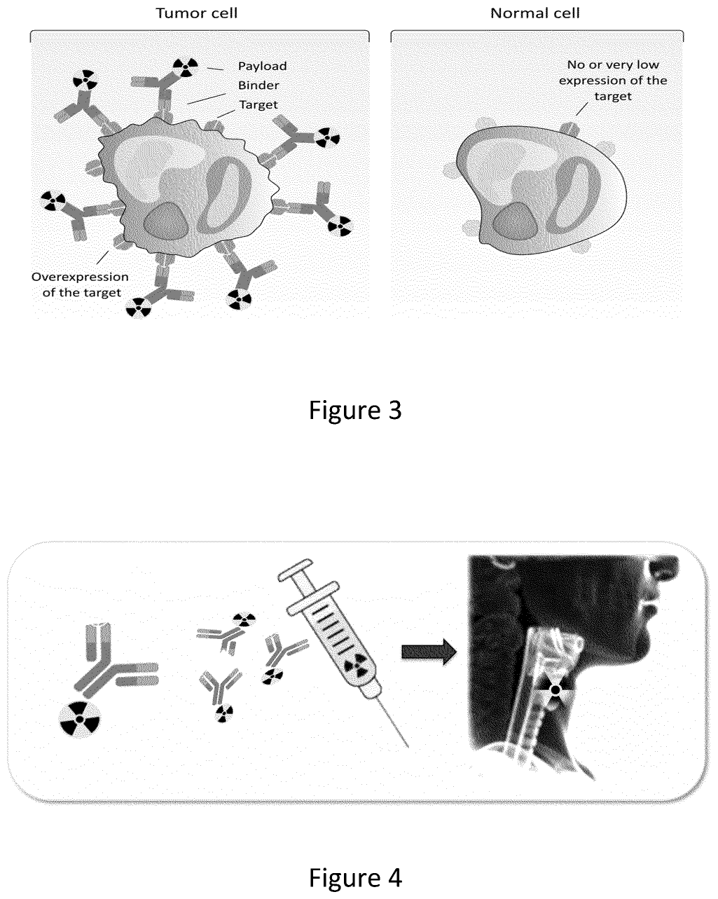

The present invention provides new enhanced binding proteins, which bind human CD44v6. The binding proteins of the invention has been evaluated for binding affinity and therapeutic effect, and it has been determined that they provide enhanced performance compares to other binding proteins in the art. One object of the present disclosure is to provide novel enhanced tumor targeting agents for improved therapy, such as radiotherapy, of cancer, to be able to find and characterize tumor/cancer cells, and also destroy them. Binding proteins may be joined/linked to one or more agents as payload as illustrated in FIG. 2, and by recognizing and binding a target present/overexpressed on tumor cells, they may either locate the tumor cell if the agent is e.g. an imaging or detectable agent, or they may kill the target cell by carrying a therapeutic agent, such as one or more radioisotopes, where radiation is the effector, as illustrated in FIG. 3, where the radioisotope conjugated binding proteins may be referred to as radiopharmaceuticals. The binding proteins are introduced into the body by various means (such as injection or ingestion) localize the specific antigen (CD44v6), bind the receptor, stay on tumor cell surface or become internalized into the tumor cell, and expose their payload of radioisotopes to the tissue, where the radiation emitted by the radioisotopes destroys the cancer cells, which is referred to as molecular radiotherapy (MRT) or radionuclide therapy (RNT).

The binding proteins of the present disclosure may be used to treat a number of disorders which are linked to expression/overexpression of the present target antigen, CD44v6, as discussed above and below. Particularly, subject suffering from thyroid cancer, especially advanced thyroid carcinoma (TC), including anaplastic (ATC) and iodine refractory TC, could benefit enormously from these treatments, as they are orphan diseases resistant to standard cancer treatments and hence no efficacious treatments are available. Thyroid cancer is in most cases a curable disease by surgical treatment followed by adjuvant treatment with radioactive iodine. There are however a number of cases that are refractory to current treatments, and these patients have very poor prognosis. The median survival after diagnosis is only 5 months for ATC, and thus the unmet clinical need is enormous. It has been established that CD44v6 is highly expressed on the tumor cells in a substantial proportion of these patients, and thus a relevant target. As the tumor cells express CD44v6, advanced thyroid carcinoma mat be treated with an effective CD44v6-targeting radiopharmaceutical for molecular radiotherapy, as illustrated in FIG. 4. The binding proteins of the present disclosure, such as CD44v6 specific mAbs, are radionuclide labeled to bring therapeutic quantities of radioactivity to CD44v6 expressing tumor cells while sparing normal, non-CD44v6 expressing tissues. In some embodiments, the radiolabeled binding protein conjugate contains a DOTA chelate with the radionuclide ¹⁷⁷Lu for therapy or ¹¹¹In for imaging. ¹¹¹In/¹⁷⁷Lu-DOTA is a well characterized theranostic pair complex, and Lutathera (¹⁷⁷Lu-DOTATATE) is approved in both US and EU for treatment of neuroendocrine cancer.

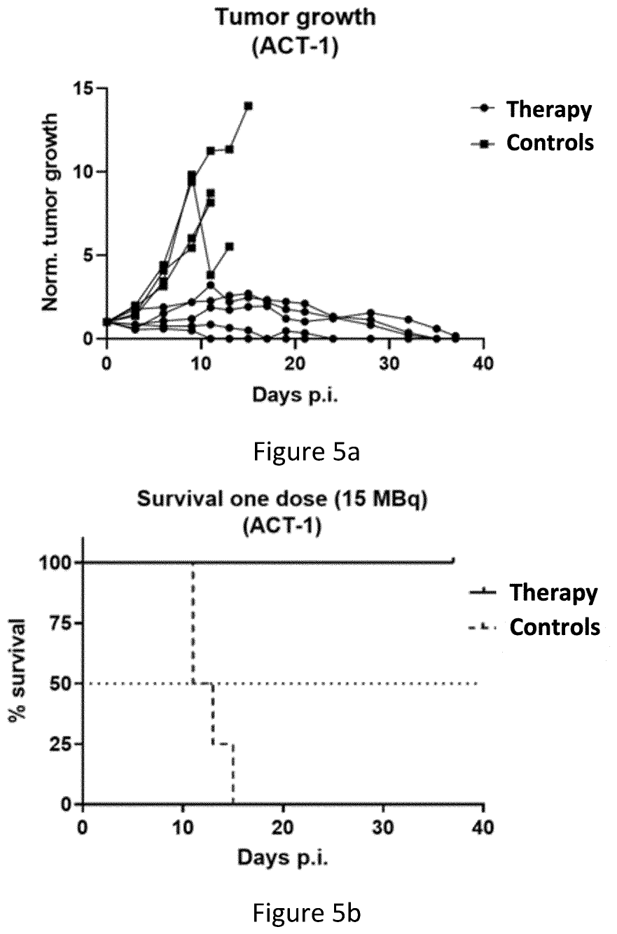

The target is previously clinically validated for radioimmunotherapy, having a tumor specific uptake, and being well tolerated. Experimental data, as summarized herein, is further set out in more detail in the example section below. PK/PD studies in mice have been performed and suggest suitable half-life, normal tissue distribution and tumor targeting capacity of the binding proteins. The experimental data also confirms dramatic effect with no observed toxicity in ATC-bearing mice, as shown in FIG. 5a, showing tumor volume in the mice up to 40 days of treatment with one dose of a radiopharmaceutical of the present disclosure (having ¹⁷⁷Lu as therapeutic agent), and the survival after one such dose in FIG. 5b. A clear and thoroughly characterized antigen-specific binding of the present binding proteins with no indications of off-target binding or cross reactivity has been demonstrated. Radioconjugates have been evaluated in three species (mouse, rabbit, cynomolgus), and dosimetric evaluations have demonstrated a favorable dosimetry, with bone marrow being the dose limiting organ, as expected. These studies also validate low CD44v6-specific normal tissue uptake in studies in rabbit and cynomolgus monkey, with no radioactivity accumulation normal tissue or active uptake in bone marrow, data not shown.

Experimental data of target binding specificity testing, as set out in more detail in the example section below, show binding of the binding proteins, and conjugated (radiolabeled) binding proteins, with high specificity and affinity to CD44v6, with low risk of off-target binding, where in silico and in vitro evaluations of off target binding and selectivity showed no such occurrence. Toxicology investigations in silico, in vitro, and in vivo using three different animal models demonstrate no toxic effects from corresponding therapeutic levels of a non-radioactive antibody, low risk of antibody-mediated effects such as T-cell activation, and low risk of off target binding. Pharmacokinetic evaluations in three different animal models also demonstrate feasible dosimetry and pharmacokinetic properties of the conjugate. SPR measurements demonstrates specific binding to CD44v6 with low nanomolar affinity (with and without DOTA-conjugation), mapping of the binding epitope, validated by SPR measurements, show that the binding proteins bind the epitope as defined by SEQ ID NO: 7. Species specificity evaluation, demonstrating binding to target in rabbit-, cynomolgus- and human CD44v6-peptides, validated by SPR measurements.

Thus, the binding proteins of the present invention may be linked/joined to an agent for imaging and/or therapy. As an example, ¹⁷⁷Lu-conjugated binding proteins could provide efficacious treatment of CD44v6-expressing radioiodine refractory thyroid cancers. Such conjugated binding proteins has been shown to bind CD44v6 with high affinity as demonstrated by radio-immunoassays on cultured thyroid cancer cells and squamous cell carcinoma cells. Real-time kinetic measurements on cultured thyroid cancer cells, demonstrates specific binding of said conjugated binding proteins to CD44v6 with high affinity on antigen-positive cells, and no binding to antigen-negative cells. In vivo biodistribution evaluations of ¹²⁵I/¹⁷⁷Lu-conjugated binding proteins in five and three xenografted mice models of varying tumor target expression respectively, showed antigen-dependent tumor uptake and no off-target binding.

Pharmacodynamic investigation therapy data on a ¹⁷⁷Lu-conjugated antibody in thyroid cancer mouse xenografts demonstrated ¹⁷⁷Lu-dependent and antigen-specific therapeutic effects (reduced tumor growth and even complete remissions), with no observed toxicity, at a single dose of 16.5 MBq/50 μg radiolabeled antibody. Data from two thyroid cancer mouse models show dose-dependent and antigen-specific therapeutic effects, with no signs of toxicity (i.e. no mouse weight loss or altered behavior). Experimental data has further shown radiodelivery potency corresponding to and also surpassing the clinically tested monoclonal antibody BIWA-4 in terms of cellular uptake in vitro and in vivo. Besides improved antigen affinity, a more suitable radionuclide label and a fully human format, the introduced LALA mutations of the binding proteins ensure lack of ADCC/CDC functions and decrease in vivo off-target uptake, for example in liver. SPR measurements of binding proteins of the present disclosure demonstrates severely reduced FcγR1-binding for silenced ADCC/CDC of the LALA constructs. These improvements provide clear advantages over previous humanized antibody-based CD44v6-targeted radiopharmaceuticals. Even though ¹⁷⁷Lu has been used as the radioisotope for therapy in the present experiments, any other radioisotope suitable for use in such treatments could be used, and is expected to give similar results.

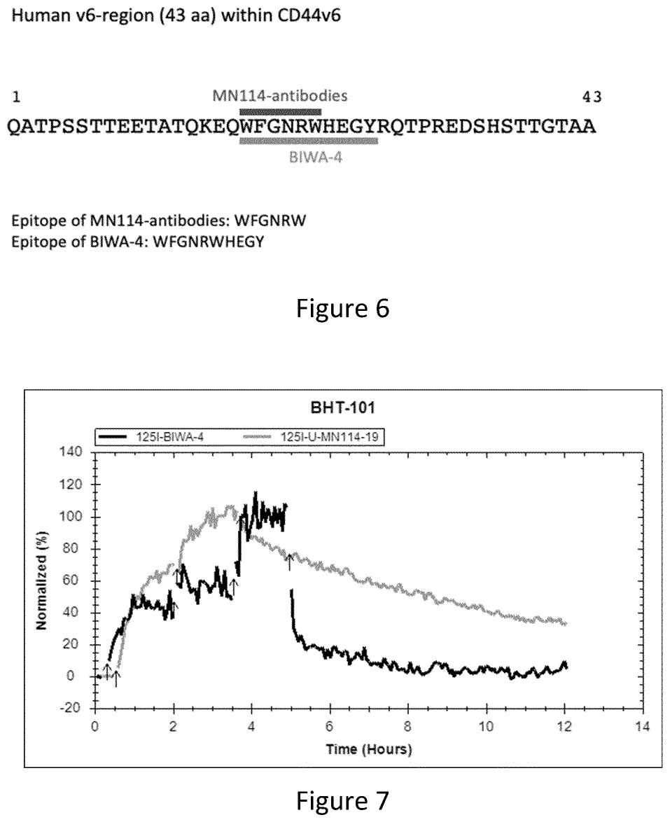

Accordingly, the present invention provides new enhanced binding proteins, which bind human CD44v6. Exon 6 of the human CD44-gene has the amino acid sequence QATPSSTTEETATQKEQWFGNRWHEGYRQTPREDSHSTTGTAA (SEQ ID NO: 115). The binding proteins are specific to an epitope coded by exon v6 of CD44, particularly to an epitope of the amino acid sequence WFGNRW (SEQ ID NO: 7). The binding proteins of the invention comprises a binding domain of an antibody, the binding domain comprising a heavy chain variable domain (VH) and a light chain variable domain (VL), or derivatives thereof, the VH and VL each comprising three complementarity determining regions (CDRs), i.e. a total of six CDRs, where the binding protein specifically binds CD44v6, and more particularly, they recognise an epitope of CD44v6 as defined by SEQ ID NO: (7). In some embodiments, the invention relates to 21 binding protein variants, which have similar sequences and all recognize the same epitope. The epitope bound by the present binding proteins, as compared to the BIWA-4 epitope WFGNRWHEGY (SEQ ID NO: 5), is illustrated in FIG. 6. Thus, the binding proteins may comprise a binding domain of an antibody comprising a heavy chain variable domain (VH) and a light chain variable domain (VL), or any derivatives thereof, wherein the derivatives may be e.g. single-domain antibodies, comprising a single monomeric variable antibody domain. In some embodiments the binding proteins are IgG antibodies devoid of light chains, which consist of two heavy chains attached to variable domains (V_{H}H).

The light and heavy chain variable domains comprise 3 CDRs each: the light chain variable domain comprises VLCDR1, VLCDR2 and VLCDR3, and the heavy chain variable domain comprises VHCDR1, VHCDR2 and VHCDR3. The six CDRs have the following amino acid sequences:
VHCDR1 as defined by SEQ ID NO: 1;
VHCDR2 as defined by SEQ ID NO: 2;
VHCDR3 as defined by SEQ ID NO: 3;
VLCDR1 as defined by SEQ ID NO: 4;
VLCDR2 as defined by X₁AS, where X₁ may be T, A, or S;
VLCDR3 as defined by SEQ ID NO: 6.
   wherein VHCDR3 and VLCDR3 are identical in all 21 binding protein variants, while VHCDR1, VHCDR2, VLCDR1 and VLCDR2 comprises some variations, as indicated in Table 1 below. Besides the CDRs according to SEQ ID NO: 1-6, the invention also encompass CDR sequences having 95% or more, such as 96%, 97%, 98%, 99% or more, identity thereto that also bind the same epitope of CD44v6.

TABLE 1

| CDR | SEQ ID NO | SEQUENCE |
|---|---|---|
| VHCDR1 | SEQ ID NO: 1 | GFX₃FX₅X₆X₇A |
| VHCDR2 | SEQ ID NO: 2 | ISX₃X₄GX₆ST |
| VHCDR3 | SEQ ID NO: 3 | ARHYYSDSDYRSSAAMDY |
| VLCDR1 | SEQ ID NO: 4 | QX₂₁X₄X₅Y |
| VLCDR2 | N/A | X₁AS |
| VLCDR3 | SEQ ID NO: 6 | QQDLYLLT |

The sequences of the CDRs of the binding proteins are shown in Table 1 in one letter amino acid code, where VHCDR1, VHCDR2, VLCDR1 and VLCDR2 comprises variations in the sequence between the 21 binding protein variants, as indicated by X. In VHCDR1, X₃ may be S or T, X₅ may be S, R or G, X₆ may be S or N, and X₇ may be Y or F. In VHCDR2 X₃ may be A or G, X₄ may be S or G, and X₆ may be T, S, Y, R or G. In VLCDR1 X₂ may be S, N or T, X₄ may be A, S or G, and X₅ may be S or N. In VLCDR2 X₁ may be A, S or T, such that VLCDR2 is AAS, SAS or TAS.

In some aspects, some CDRs are surrounded by specific amino acids, referred to as framework amino acids (faa), which are conserved between the different binding proteins, with some small variations. Accordingly, the VHCDR1, VHCDR2 and VLCDR2 of the binding protein may be present next to specific framework amino acids, wherein the CDR and framework amino acid sequences are selected from the group comprising:
VHCDR1 and faa defined by SEQ ID NO: 8;
VHCDR2 and faa as defined by SEQ ID NO: 9;
VLCDR2 and faa as defined by SEQ ID NO: 10;
   as indicated in Table 2 below. Besides the CDRs+faa according to SEQ ID NO: 8-10, the invention also encompass CDR sequences having 95% or more, such as 96%, 97%, 98%, 99% or more, identity thereto that also bind the same epitope of CD44v6.

TABLE 2

| CDR | SEQ ID NO | SEQUENCE |
|---|---|---|
| VHCDR1 and faa | SEQ ID NO: 8 | GFX₃FX₅X₆X₇AMS |
| VHCDR2 and faa | SEQ ID NO: 9 | X₁ISX₄X₅GX₇STX₁₀ |
| VLCDR2 and faa | SEQ ID NO: 10 | X₁ASX₄ |

For VHCDR1 and faa, the sequence is the same as for VHCDR1, but with two additional framework amino acids at the end, MS. In VHCDR2 and faa, an additional faa is added at the beginning and one at the end of the sequence, where X₁ may be A or T, and X₁₀ may be Y or F. The remaining unknowns in the middle correspond to the VHCDR2 sequence of Table 1 above. In VLCDR2 and faa, the additional faa X₄ may be S, T, N or I, the remaining unknowns correspond to the sequence of Table 1 above.

In some aspects, 21 different binding proteins are presented, having the individual CDR combinations as presented in Tables 3-4 below, where U-MN114-19 is the parental clone, and the other 20 binding proteins affinity maturated versions thereof.

TABLE 3

| Clone name | VHCDR1 | VHCDR2 |
|---|---|---|
| U-MN114-19 | GFTFSSYA (SEQ ID NO: 12) | ISGSGGST (SEQ ID NO: 32) |
| AL-MN114-465 | GFSFGSYA (SEQ ID NO: 11) | ISAGGSST (SEQ ID NO: (19) |
| AL-MN114-451 | GFTFSSYA (SEQ ID NO: 12) | ISAGGYST (SEQ ID NO: 22) |
| AL-MN114-452 | GFSFSSYA (SEQ ID NO: 13) | ISAGGSST (SEQ ID NO: 19) |
| AL-MN114-460 | GFSFGSYA (SEQ ID NO: 11) | ISAGGSST (SEQ ID NO: 19) |
| AL-MN114-423 | GFTFSSYA (SEQ ID NO: 12) | ISAGGTST (SEQ ID NO: 21) |
| AL-MN114-145 | GFSFSSFA (SEQ ID NO: 14) | ISAGGSST (SEQ ID NO: 19) |
| AL-MN114-429 | GFSFSSFA (SEQ ID NO: 14) | ISAGGSST (SEQ ID NO: 19) |
| AL-MN114-435 | GFTFSSYA (SEQ ID NO: 12) | ISAGGSST (SEQ ID NO: 19) |
| AL-MN114-439 | GFSFGSFA (SEQ ID NO: 15) | ISAGGSST (SEQ ID NO: 19) |
| AL-MN114-71 | GFSFRSFA (SEQ ID NO: 16) | ISGGGSST (SEQ ID NO: 20) |
| AL-MN114-79 | GFTFRSFA (SEQ ID NO: 17) | ISAGGSST (SEQ ID NO: 19) |
| AL-MN114-93 | GFSFGSFA (SEQ ID NO: 15) | ISAGGSST (SEQ ID NO: 19) |
| AL-MN114-126 | GFTFSSYA (SEQ ID NO: 12) | ISAGGSST (SEQ ID NO: 19) |
| AL-MN114-42 | GFTFSSYA (SEQ ID NO: 12) | ISGGGYST (SEQ ID NO: 23) |
| AL-MN114-18 | GFSFGSFA (SEQ ID NO: 15) | ISGGGSST (SEQ ID NO: 20) |
| AL-MN114-19 | GFSFGSYA (SEQ ID NO: 11) | ISGGGSST (SEQ ID NO: 20) |
| AL-MN114-132 | GFTFSSYA (SEQ ID NO: 12) | ISGGGSST (SEQ ID NO: 20) |
| AL-MN114-23 | GFSFRNFA (SEQ ID NO: 18) | ISGGGGST (SEQ ID NO: 24) |
| AL-MN114-14 | GFTFSSYA (SEQ ID NO: 12) | ISGSGRST (SEQ ID NO: 25) |
| AL-MN114-444 | GFTFRSFA (SEQ ID NO: 17) | ISGGGSST (SEQ ID NO: 20) |

Table 3 shows the CDRs H1 and H2 for the respective 21 different binding proteins. The CDR for H3 (VHCDR3) for all 21 binding proteins is defined by sequence ARHYYSDSDYRSSAAMDY (SEQ ID NO:3).

TABLE 4

| Clone name | VLCDR1 | VLCDR2 | VLCDR3 |
|---|---|---|---|
| U-MN114-19 | QSISSY (SEQ ID NO: 26) | AAS (SEQ ID NO: N/A) | QQDLYLLT (SEQ ID NO: 6) |
| AL-MN114-465 | QSISSY (SEQ ID NO: 26) | TAS (SEQ ID NO: N/A) | QQDLYLLT (SEQ ID NO: 6) |
| AL-MN114-451 | QSIGSY (SEQ ID NO: 27) | TAS (SEQ ID NO: N/A) | QQDLYLLT (SEQ ID NO: 6) |
| AL-MN114-452 | QSIGSY (SEQ ID NO: 27) | SAS (SEQ ID NO: N/A) | QQDLYLLT (SEQ ID NO: 6) |
| AL-MN114-460 | QSIGSY (SEQ ID NO: 27) | SAS (SEQ ID NO: N/A) | QQDLYLLT (SEQ ID NO: 6) |
| AL-MN114-423 | QSISSY (SEQ ID NO: 26) | AAS (SEQ ID NO: N/A) | QQDLYLLT (SEQ ID NO: 6) |
| AL-MN114-145 | QSIGSY (SEQ ID NO: 27) | SAS (SEQ ID NO: N/A) | QQDLYLLT (SEQ ID NO: 6) |
| AL-MN114-429 | QSISSY (SEQ ID NO: 26) | AAS (SEQ ID NO: N/A) | QQDLYLLT (SEQ ID NO: 6) |
| AL-MN114-435 | QSISSY (SEQ ID NO: 26) | AAS (SEQ ID NO: N/A) | QQDLYLLT (SEQ ID NO: 6) |
| AL-MN114-439 | QSISSY (SEQ ID NO: 26) | AAS (SEQ ID NO: N/A) | QQDLYLLT (SEQ ID NO: 6) |
| AL-MN114-71 | QSISSY (SEQ ID NO: 26) | AAS (SEQ ID NO: N/A) | QQDLYLLT (SEQ ID NO: 6) |
| AL-MN114-79 | QSIANY (SEQ ID NO: 28) | AAS (SEQ ID NO: N/A) | QQDLYLLT (SEQ ID NO: 6) |
| AL-MN114-93 | QSISSY (SEQ ID NO: 26) | AAS (SEQ ID NO: N/A) | QQDLYLLT (SEQ ID NO: 6) |
| AL-MN114-126 | QSISSY (SEQ ID NO: 26) | AAS (SEQ ID NO: N/A) | QQDLYLLT (SEQ ID NO: 6) |
| AL-MN114-42 | QTIGSY (SEQ ID NO: 29) | SAS (SEQ ID NO: N/A) | QQDLYLLT (SEQ ID NO: 6) |
| AL-MN114-18 | QTIASY (SEQ ID NO: 30) | TAS (SEQ ID NO: N/A) | QQDLYLLT (SEQ ID NO: 6) |
| AL-MN114-19 | QSISSY (SEQ ID NO: 26) | SAS (SEQ ID NO: N/A) | QQDLYLLT (SEQ ID NO: 6) |
| AL-MN114-132 | QSISSY (SEQ ID NO: 26) | AAS (SEQ ID NO: N/A) | QQDLYLLT (SEQ ID NO: 6) |
| AL-MN114-23 | QNISSY (SEQ ID NO: 31) | SAS (SEQ ID NO: N/A) | QQDLYLLT (SEQ ID NO: 6) |
| AL-MN114-14 | QSISSY (SEQ ID NO: 26) | AAS (SEQ ID NO: N/A) | QQDLYLLT (SEQ ID NO: 6) |
| AL-MN114-444 | QSIGSY (SEQ ID NO: 27) | SAS (SEQ ID NO: N/A) | QQDLYLLT (SEQ ID NO: 6) |

Table 4 shows the CDRs L1, L2 and L3 for the respective 21 different binding proteins.

In some aspects, 21 different binding proteins are presented, having the individual CDR combinations as described above, and surrounding framework amino acids for VHCDR1, VHCDR2 and VLCDR2 as presented in Tables 5 and 6 below.

TABLE 5

| Clone name | VHCDR1 + faa | VHCDR2 + faa |
|---|---|---|
| U-MN114-19 | GFTFSSYAMS (SEQ ID NO: 122) | AISGSGGSTY (SEQ ID NO: 33) |
| AL-MN114-465 | GFSFGSYAMS (SEQ ID NO: 121) | AISAGGSSTF (SEQ ID NO: 129) |
| AL-MN114-451 | GFTFSSYAMS (SEQ ID NO: 122) | AISAGGYSTY (SEQ ID NO: 134) |
| AL-MN114-452 | GFSFSSYAMS (SEQ ID NO: 123) | AISAGGSSTF (SEQ ID NO: 129) |
| AL-MN114-460 | GFSFGSYAMS (SEQ ID NO: 121) | AISAGGSSTY (SEQ ID NO: 132) |
| AL-MN114-423 | GFTFSSYAMS (SEQ ID NO: 122) | AISAGGTSTY (SEQ ID NO: 133) |
| AL-MN114-145 | GFSFSSFAMS (SEQ ID NO: 124) | AISAGGSSTF (SEQ ID NO: 129) |
| AL-MN114-429 | GFSFSSFAMS (SEQ ID NO: 124) | AISAGGSSTY (SEQ ID NO: 132) |
| AL-MN114-435 | GFTFSSYAMS (SEQ ID NO: 122) | AISAGGSSTF (SEQ ID NO: 129) |
| AL-MN114-439 | GFSFGSFAMS (SEQ ID NO: 125) | AISAGGSSTF (SEQ ID NO: 129) |
| AL-MN114-71 | GFSFRSFAMS (SEQ ID NO: 126) | AISGGGSSTF (SEQ ID NO: 130) |
| AL-MN114-79 | GFTFRSFAMS (SEQ ID NO: 127) | TISAGGSSTF (SEQ ID NO: 135) |
| AL-MN114-93 | GFSFGSFAMS (SEQ ID NO: 125) | AISAGGSSTY (SEQ ID NO: 132) |
| AL-MN114-126 | GFTFSSYAMS (SEQ ID NO: 122) | AISAGGSSTY (SEQ ID NO: 132) |
| AL-MN114-42 | GFTFSSYAMS (SEQ ID NO: 122) | AISGGGYSTY (SEQ ID NO: 136) |
| AL-MN114-18 | GFSFGSFAMS (SEQ ID NO: 125) | AISGGGSSTY (SEQ ID NO: 131) |
| AL-MN114-19 | GFSFGSYAMS (SEQ ID NO: 121) | AISGGGSSTF (SEQ ID NO: 130) |
| AL-MN114-132 | GFTFSSYAMS (SEQ ID NO: 122) | AISGGGSSTY (SEQ ID NO: 131) |
| AL-MN114-23 | GFSFRNFAMS (SEQ ID NO: 128) | AISGGGGSTF (SEQ ID NO: 137) |
| AL-MN114-14 | GFTFSSYAMS (SEQ ID NO: 122) | AISGSGRSTF (SEQ ID NO: 138) |
| AL-MN114-444 | GFTFRSFAMS (SEQ ID NO: 127) | AISGGGSSTF (SEQ ID NO: 130) |

TABLE 6

| Clone name | VLCDR2 + faa |
|---|---|
| U-MN114-19 | AASS (SEQ ID NO: 141) |
| AL-MN114-465 | TASS (SEQ ID NO: 139) |
| AL-MN114-451 | TAST (SEQ ID NO: 142) |
| AL-MN114-452 | SASS (SEQ ID NO: 140) |
| AL-MN114-460 | SASS (SEQ ID NO: 140) |
| AL-MN114-423 | AASS (SEQ ID NO: 141) |
| AL-MN114-145 | SASN (SEQ ID NO: 143) |
| AL-MN114-429 | AASS (SEQ ID NO: 141) |
| AL-MN114-435 | AASS (SEQ ID NO: 141) |
| AL-MN114-439 | AASS (SEQ ID NO: 141) |
| AL-MN114-71 | AAST (SEQ ID NO: 144) |
| AL-MN114-79 | AAST (SEQ ID NO: 144) |
| AL-MN114-93 | AASS (SEQ ID NO: 141) |
| AL-MN114-126 | AASS (SEQ ID NO: 141) |
| AL-MN114-42 | SASI (SEQ ID NO: 145) |
| AL-MN114-18 | TASN (SEQ ID NO: 146) |
| AL-MN114-19 | SASS (SEQ ID NO: 140) |
| AL-MN114-132 | AASS (SEQ ID NO: 141) |
| AL-MN114-23 | SASI (SEQ ID NO: 145) |
| AL-MN114-14 | AASS (SEQ ID NO: 141) |
| AL-MN114-444 | SASS (SEQ ID NO: 140) |

The binding proteins of the invention may be synthesised by any method known in the art. Preferably, the binding proteins are synthesised using a protein expression system, such as a cellular expression system using prokaryotic (e.g. bacterial) cells or eukaryotic (e.g. yeast, fungus, insect or mammalian) cells. An alternative protein expression system is a cell-free, in vitro expression system, in which a nucleotide sequence encoding the binding protein is transcribed into mRNA, and the mRNA translated into a protein, in vitro. Cell-free expression system kits are widely available, and can be purchased from e.g. Thermo Fisher Scientific. Alternatively, binding proteins may be chemically synthesised in a non-biological system. Liquid-phase synthesis or solid-phase synthesis may be used to generate polypeptides which may form or be comprised within the binding protein of the invention.

The skilled person can readily produce binding proteins using appropriate methodology common in the art. In particular, the binding proteins may be recombinantly expressed in mammalian cells, such as CHO cells. A binding protein synthesised in a protein expression system may be purified using standard techniques in the art, e.g. it may be synthesised with an affinity tag and purified by affinity chromatography. If the binding protein is an antibody, it can be purified using affinity chromatography using one or more antibody-binding proteins, such as Protein G, Protein A, Protein A/G or Protein L.

As noted above, the binding proteins are antibody-based, or antibody-like, molecules. Thus, a binding protein may be a native antibody or a fragment thereof, or an artificial or synthetic antibody, or an antibody construct or derivative (e.g. a single chain antibody). In a preferred embodiment, the binding protein is a human protein (of human origin as compared to humanized), in particular a human monoclonal antibody, antibody fragment or scFv. A human binding protein may comprise VH and VL regions in which both framework and CDR regions are derived from human germline immunoglobulin sequences, and also a human constant region, if a constant region is contained in the protein. Such proteins may however include amino acids not encoded by human germline Ig sequences, for example mutations introduced by random or site-specific mutagenesis.

As detailed above, the binding proteins of the invention comprises a binding domain of an antibody, the binding domain comprising a heavy chain variable domain (or variable region) and a light chain variable domain. Thus, in a particular embodiment the binding protein of the invention comprises:
(i) a heavy chain variable domain (VH) comprising (or consisting of) the amino acid sequence set forth in any one of SEQ ID NO: 35-54 and 147, or a variant thereof; and
(ii) a light chain variable domain (VL) comprising (or consisting of) the amino acid sequence set forth in any one of SEQ ID NO: 55-74 and 148, or a variant thereof.

In some embodiments, the binding proteins comprises a heavy chains and a light chains of comprising variable and constant regions, where the binding protein of the invention comprises:
(i) a heavy chain comprising (or consisting of) the amino acid sequence set forth in any one of SEQ ID NO: 34, 75-93 and 149, or a variant thereof; and
(ii) a light chain comprising (or consisting of) the amino acid sequence set forth in any one of SEQ ID NO: 94-113 and 150, or a variant thereof.

A variant is defined as sequences having 80% or more, such as 85%, 90%, 95% or more, identity thereto. This is with the proviso that the CDR sequences of the variants are unaltered in view of the antibody variant defined by the VH or VL domain, i.e. comprise no sequence variation, or wherein the sequence variation of the CDR amino acid sequences are at most 5%, such as 4%, 3%, 2%, 1% or less, or is such that the CDR sequence variations have a sequence identity to the defined sequences that is at least 95%, such as 96%, 97%, 98%, 99%, or more. Binding proteins with variants of the sequences of the variable and/or constant domains are functional variants, having the activities described above (i.e. they specifically bind the defined epitope of CD44v6). Variant sequences may be modified relative to the native sequences by substitution, insertion and/or deletion of one or more amino acids.

Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet., 16, (6) pp 276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32 (5): 1792-1797, 2004) may be used for multiple sequence alignments, though any other appropriate programme may be used. Whether the alignment is pairwise or multiple, it must be performed globally (i.e. across the entirety of the reference sequence) rather than locally. Sequence alignments and % identity calculations may be determined using for instance standard Clustal Omega parameters: matrix Gonnet, gap opening penalty 6, gap extension penalty 1. Alternatively, the standard EMBOSS Needle parameters may be used: matrix BLOSUM62, gap opening penalty 10, gap extension penalty 0.5. Any other suitable parameters may alternatively be used.

In some aspects, the present disclosure encompasses conjugated binding proteins, such as antibody drug conjugates. Thus, the present disclosure provides a conjugated binding protein comprising: (i) at least one binding protein as described above and below; and (ii) at least one agent, wherein the at least one agent is joined to the binding protein, wherein the binding protein and agent are directly or indirectly joined.

In a further aspect, the invention provides a pharmaceutical composition comprising a binding protein of the invention, as described above, or a conjugated binding protein, as described above, or an engineered CAR-cell, as described above. In addition, the pharmaceutical composition also comprises at least one pharmaceutically acceptable carrier or excipient. As used herein, “pharmaceutically acceptable carrier or excipient” includes any and all solvents, dispersion media, coatings, antibacterial and antifungal agents, isotonic and absorption delaying agents and the like that are physiologically compatible.

Preferably, the carrier or excipient is suitable for parenteral, e.g. intradermal, intravenous, intramuscular or subcutaneous administration (e.g. by injection or infusion). Depending on the route of administration, the binding protein or conjugated binding protein may be coated in a material to protect them from the action of acids and other natural conditions that may inactivate or denature it.

Preferred pharmaceutically-acceptable carriers comprise aqueous carriers or diluents. Examples of suitable aqueous carriers that may be employed in the pharmaceutical compositions, kits and products include water, buffered water and saline. Examples of other carriers include ethanol, polyols (such as glycerol, propylene glycol, polyethylene glycol, and the like), and suitable mixtures thereof, vegetable oils and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, sodium chloride and the like.

The binding proteins, conjugated binding proteins, or pharmaceutical compositions comprising the binding proteins or conjugated binding proteins may be administered via one or more routes of administration using one or more of a variety of methods known in the art.

As will be appreciated by the skilled artisan, the route and/or mode of administration will vary depending upon the desired results. Preferred routes of administration include intravenous, intramuscular, intradermal, intraperitoneal, subcutaneous, spinal or other parenteral routes of administration, for example by injection or infusion, e.g. directly to the site of a tumour. The phrase “parenteral administration” as used herein means modes of administration other than enteral and topical administration, usually by injection. Alternatively, a non-parenteral route may be used, such as a topical, epidermal or mucosal route of administration. Local administration is preferred, including peritumoral, juxtatumoral, intratumoral, intralesional, perilesional, intra cavity infusion, intravesicle administration, and inhalation. However, the antigen binding protein, conjugate of engineered cells may also be administered systemically.

A suitable dosage of a specific binding protein, conjugated binding protein, or pharmaceutical composition of the invention may be determined by a skilled medical practitioner. Actual dosage levels of the active ingredients in the pharmaceutical compositions and products of the present invention may be varied so as to obtain an amount of the active ingredient which is effective to achieve the desired therapeutic response for a particular subject, i.e. patient, without being toxic to the patient. The selected dosage level will depend upon a variety of pharmacokinetic factors including the activity of the particular protein/conjugate employed, the route of administration, the time of administration, the rate of excretion of the protein, the duration of the treatment, other drugs, compounds and/or materials used in combination with the particular compositions employed, the age, sex, weight, condition, general health and prior medical history of the patient being treated, and like factors well known in the medical arts.

A suitable dose of a binding protein or conjugated binding protein of the invention may be, for example, in the range of from about 0.1 μg/kg to about 100 mg/kg body weight of the patient to be treated. For example, a suitable dosage may be from about 1 μg/kg to about 20 mg/kg body weight per dosing or from about 10 μg/kg to about 10 mg/kg body weight per dosing. For a conjugated binding protein carrying radioisotopes, the doses may be given at certain intervals, such as bi-weekly (every fortnight). For other purposes, shorter intervals, such as daily doses, may be more suitable. Suitable intervals for different types of treatments would be apparent to the skilled person.

Dosage regimens may be adjusted to provide the optimum desired response (e.g. a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The binding protein or conjugate/composition may be administered in a single dose or in multiple doses. The multiple doses may be administered via the same or different routes and to the same or different locations. Alternatively, they can be administered as a sustained release formulation, in which case less frequent administration is required. Dosage and frequency may vary depending on the half-life of the administered species in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms of disease. In an exemplary dosage regime, the conjugated binding protein is administered to the subject once a week, once a fortnight or once every three weeks, in a cycle repeated from 2 to 10 times.

Even though the splice variant CD44v6 is mainly expressed in tumor cells, showing homogenous expression of CD44v6 in many cancers, and only among normal tissues being expressed in a subset of epithelial tissues (keratinocytes), a pretargeting treatment scheme may be used. In a pretargeting treatment scheme, the binding protein itself does not carry the payload (agent), but it is carried by a second molecule. The binding protein, which in this embodiment comprises a molecular attachment tag is administered to a subject, and allowed to bind the target (CD44v6). The unbound binding proteins are then allowed to be cleared from the system naturally or upon injection of a clearing agent, and after the unbound binding proteins have left the circulation, the second molecule carrying the payload, i.e. binding the therapeutic agent, is administered. The second binding molecule binds the molecular attachment tag of the target bound binding protein, hence delivering the payload to the target. Thus, a method of treating a subject in need thereof is provided, the method comprising administering a first binding protein comprising a molecular attachment tag, allowing any unbound binding proteins to leave the circulation of the subject, administering a therapeutically effective amount of a second molecule, wherein the second molecule is joined to a therapeutic agent, and wherein the second molecule bind the first binding protein, thereby delivering the therapeutic agent to the CD44v6 epitope bound by first binding protein.

The content of this disclosure thus enables treatment, imaging and diagnosis of disorders linked to CD44v6 expression, such as cancer, by administering conjugated binding proteins of the invention. In the drawings and specification, there have been disclosed exemplary aspects of the disclosure. However, many variations and modifications can be made to these aspects without substantially departing from the principles of the present disclosure. Thus, the disclosure should be regarded as illustrative rather than restrictive, and not as being limited to the particular aspects discussed above. Accordingly, although specific terms are employed, they are used in a generic and descriptive sense only and not for purposes of limitation.

The description of the example embodiments provided herein have been presented for purposes of illustration. The description is not intended to be exhaustive or to limit example embodiments to the precise form disclosed, and modifications and variations are possible in light of the above teachings or may be acquired from practice of various alternatives to the provided embodiments. The examples discussed herein were chosen and described in order to explain the principles and the nature of various example embodiments and its practical application to enable one skilled in the art to utilize the example embodiments in various manners and with various modifications as are suited to the particular use contemplated. The features of the embodiments described herein may be combined in all possible combinations of methods, products, and systems. It should be appreciated that the example embodiments presented herein may be practiced in any combination with each other. It should also be noted that the word “comprising” does not necessarily exclude the presence of other elements or steps than those listed and the words “a” or “an” preceding an element do not exclude the presence of a plurality of such elements. It should further be noted that any reference signs do not limit the scope of the claims, that the example embodiments may be realized in the broadest sense of the claims.

### EXAMPLES

In brief, CD44v6-binding antibodies have been selected and affinity maturated. The candidates were epitope-mapped and extensively evaluated both in vitro and in vivo, validated radionuclide labeling, in vivo kinetics and dosimetry as well as performed successful molecular radiotherapy experiments in several thyroid carcinoma mouse xenografts.

The tables 7-9 below summarize the relevant in silico, in vitro and in vivo experiments performed on selected MN114 antibodies of the present disclosure, after selections and format conversions, wherein selected example experiments are outlined in more detail below. Related to pharmacology, toxicology and pharmacokinetics, many of these experiments are also described in more detail in the sections below. If nothing else is stated in the following, the antibody format is IgG1 LALA.

TABLE 7
Experiments performed on selected non-radionuclide labeled MN114 antibodies

| Experiment, | | | |
|---|---|---|---|
| in silico | | | |
| & in vitro | Aim | Methods | Result |
| Binding | Assess CD44v6 binding | SPR, ELISA | Clear target binding for all selected clones and |
| specificity, | specificity and affinity for | | formats. Improved affinity to CD44v6 peptide for |
| affinity | various clones and formats | | selected AL-MN114 clones compared to |
| | | | parental U-MN114-19. Selected MN114 clones |
| | | | displayed higher affinity to target than BIWA4. |
| Affinity, | Affinity and stability of | SPR, HPLC | DOTA conjugation does not alter affinity. No |
| stability | DOTA-conjugated Ab | SEC, Nano | aggregation over time detected. Generally |
| | stored at −80° C., −20° C. | DSF, SDS | stable. HPLC SEC: HMW component observed |
| | and +4° C. and analyzed at | PAGE, DLS | at −20° C. |
| | day 0, 7, 16 and 23. | | |
| Affinity | CD44v6 species specificity | SPR | Abs not cross reactive to mouse, sheep or pig |
| | | | CD44v6, but cross reactive to CD44v6 in rabbit |
| | | | and cynomolgus monkey. BIWA-4 only cross |
| | | | reactive to cynomolgus. |
| Affinity | FcRn affinity, human, | SPR | AL-MN114-465 affinity to human, mouse and |
| | mouse, rabbit | | rabbit FcRn at pH 6, respectively: 1.1E−07, |
| | | | 3.4E−07, 2.1E−07. |
| Affinity | FcyR affinity | SPR | Drastically depleted FcyR1 binding for AL- |
| | | | MN114-465 (K_{D}: 2.7E−08M) compared to |
| | | | Herceptin (K_{D}: 1.8E−10M) |
| Sequencing | Detailing the different | SPR | Defined common six aa epitope for MN114 |
| and | variants | | clones. Partly overlapping with the ten aa |
| epitope | | | epitope for BIWA4. |
| mapping | | | |
| Biophysical | Analysis of samples stored | HPLC SEC, | No red flags. Monomeric. Defined melting |
| analysis | at 4° C. 2-3 months | Nano DSF, | points. Highly similar SDS PAGE & PI. |
| | | SDS PAGE, | |
| | | IEF | |
| In silico | Assess potential T-cell | In silico | No major increase in immunogenicity risk. In |
| Immuno- | epitopes | | this comparison the MN114 candidates, as well |
| genicity | | | as BIWA4, ranked closer to Avastin and |
| assessment | | | Herceptin, both showing low clinical |
| | | | immunogenicity, compared to Adalimumab. |
| In silico | In silico analysis of epitope | In silico | low probability of cross-reactivity for MN114 |
| evaluation | sequence and potential | | clones |
| of | cross binding to other | | |
| selectivity | proteins | | |
| Off target | Binding to “cross-reactivity | ELISA | Signals for U-MN114-19 from the cross- |
| binding | antigens” | | reactivity antigen panel assay remained low or |
| | | | moderate. |
| Cell | Assess the cell viability | XTT cell | Viability studies demonstrated no effect from |
| viability | effects of non-radiolabeled | viability | AL-MN114-465 or U-MN114-19 72 h post |
| | Abs (1-10 000 nM) on | assay | treatment start. |
| | cultured human | | |
| | immortalized keratinocytes | | |
| | and thyroid cancer cells | | |
| Molecular | Assess potential molecular | Olink PLA | No significant effects on the 92 various markers |
| effects of | effects on keratinocytes | analysis | in the Olink Target 96 Oncology II panel at 24 |
| antibody | from antibody binding | | and 72 h post treatment with 1 μM antibody |
| binding | | | |

TABLE 8
In vitro experiments performed on selected radionuclide-labeled MN114 antibodies

| | | | Radio- | |
|---|---|---|---|---|
| Experiment | Aim | Methods | nuclide | Result |
| Affinity | Assess binding | Ligand- | ¹²⁵I | Low nanomolar affinity confirmed for all |
| | affinity in real time | Tracer | | variants. Top MN114 clones |
| | on cultured cancer | | | demonstrated higher affinity compared to |
| | cells | | | BIWA4 on selected cells |
| Affinity | Assess binding | Ligand- | ¹⁷⁷Lu, ¹¹¹In | Low nanomolar affinity confirmed, |
| | affinity in real time | Tracer | | improved cellular retention for selected |
| | on cultured cancer | | | AL-MN114-clones compared to U- |
| | cells | | | MN114-19 |
| Specificity | To confirm | Cell-based | ¹²⁵I | Antigen specific binding on cells |
| | specificity to target | competition | | confirmed. Top MN114 clones |
| | on cells | on Ligand- | | outperformed BIWA4 on selected cells |
| | | Tracer | | |
| Specificity | To confirm | Blocking | ¹⁷⁷Lu, | Antigen specific binding on cells |
| | specificity to target | radio-assays | ¹²⁵I | confirmed on several cell lines with |
| | on cells | | | varying antigen expression levels |
| Stability: | Assess stability of | Serum | ¹⁷⁷Lu | Stability of radioconjugate confirmed |
| radio- | ¹⁷⁷Lu-labeling over | (human) | | |
| labeling | time | stability at | | |
| | | 37° C., EDTA- | | |
| | | challenge | | |

TABLE 9
In vivo experiments performed on selected radionuclide-labeled MN114 antibodies

| | | | Xeno- | |
|---|---|---|---|---|
| | | Radio- | grafted | |
| Experiment | Aim | nuclide | cell line | Result |
| Therapy, | ¹⁷⁷Lu-therapy vs. | ¹⁷⁷Lu | ACT-1 | All treated mice cured (15 MBq/50 μg |
| xenografted mice | Non-radiolabeled | | | antibody), no signs of toxicity |
| | Ab | | | |
| Therapy, | ¹⁷⁷Lu-therapy vs. | ¹⁷⁷Lu | BHT-101 | Reduced tumor growth, increased median |
| xenografted mice | ¹⁷⁷Lu-unspecific Ab | | | survival and maximum survival (7 MBq/ |
| | | | | 50 μg antibody) compared to ¹⁷⁷Lu-B1-8 |
| | | | | (Isotope control antibody) |
| Therapy, | ¹⁷⁷Lu-therapy vs. | ¹⁷⁷Lu | BHT-101 | All treated mice cured (10 MBq/50 μg), |
| xenografted mice | ¹⁷⁷Lu-BIWA4 | | | response rates of therapy with ¹⁷⁷Lu-AL- |
| | therapy | | | MN114-465 were superior to those of |
| | | | | ¹⁷⁷Lu-BIWA4, shortening time to both |
| | | | | partial and complete responses. |
| Biodistribution | Comparison of ¹²⁵I | ¹²⁵I, | ACT-1 | Validated in vivo tumor targeting. Identical |
| studies, dual | and ¹⁷⁷Lu and | ¹⁷⁷Lu | | blood kinetics for ¹⁷⁷Lu and ¹²⁵I-labeled |
| nuclide, | general distribution | | | conjugates, but higher tumor uptake for |
| xenografted mice | profile | | | ¹⁷⁷Lu-labeled conjugates |
| Biodistribution | Comparison of U- | ¹²⁵I | ACT-1 | Validated in vivo tumor targeting. U- |
| studies with ¹²⁵I, | MN114-19 IgG4, | | | MN114-19 demonstrated significantly |
| xenografted mice | BIWA-4 IgG4, IgG1 | | | higher tumor uptake compared to BIWA-4. |
| | LALA IgG1 and | | | Confirmed faster blood clearance for the |
| | LALA/IAHA | | | IAHA conjugate. |
| Biodistribution | U-MN114-19vs AL- | ¹⁷⁷Lu | A431, | Validated in vivo tumor targeting. |
| xenografted mice, | MN114-465in | | BHT-101 | Favorable dosimetry profile. |
| dosimetry | xenografts with | | | |
| | varying antigen | | | |
| | expression | | | |
| Titration mice | Assess effects of | ¹⁷⁷Lu | n/a | No difference in normal tissue uptake or |
| ¹⁷⁷Lu | varying doses of | | | clearance for different antibody doses (15- |
| | antibody on uptake | | | 100 μg). |
| | in e.g. liver and | | | |
| | spleen | | | |
| Blood kinetics in | Confirm blood | ¹²⁵I | n/a | Blood kinetics comparable to tumor |
| immune- | kinetic profile in | | | bearing mice. |
| competent mice | immune-competent | | | |
| ¹²⁵I | animals | | | |
| Imaging | SPECT/CT | ¹⁷⁷Lu | ACT-1, | Tumors clearly visible at 14 days p.i. |
| xenografted mice | | | BHT-101 | (ACT-1) and 5 days p.i. (BHT-101) |
| Rabbit | Pilot of rabbit as | ¹⁷⁷Lu | n/a | Comparable blood kinetics to mouse and |
| biodistribution | model system | | | cynomolgus. No active bone marrow |
| pilot, ¹⁷⁷Lu | | | | uptake. Low uptake in normal tissue. |
| Cynomolgus | ⁶⁸Ga PET/CT | ⁶⁸Ga | n/a | Comparable blood kinetics to mouse and |
| imaging | imaging study | | | rabbit. No accumulation in normal tissue |
| | | | | during assessed time span. |

TABLE 10
List of proteins and peptides used in various examples below

| Name | Short name | Source | Characteristics |
|---|---|---|---|
| Human CD44v6 | CD44v6 | ProMab, custom | Produced in HKB11 cells, Fc- |
| iso4-Fc | | ordered | fused, C-terminal Hisx10. |
| Human CD44v6 | Bio-CD44v6 | ProMab, custom | Produced in HKB11 cells, Fc- |
| iso4-Fc | | ordered | fused, C-terminal Hisx10. |
| | | | Chemically biotinylated. |
| Human CD44 iso6- | CD44 | ProMab, custom | Produced in HKB11 cells, Fc- |
| Fc | | ordered | fused, C-terminal Hisx10. |
| Human CD44 iso6- | Bio-CD44 | ProMab, custom | Produced in HKB11 cells, Fc- |
| Fc | | ordered | fused, C-terminal Hisx10. |
| | | | Chemically biotinylated. |
| Human v6-peptide | Hm v6-peptide | Bachem #4108947 | 43 amino acids, peptide |
| | | | synthesized, N-terminal biotin |
| Cynomolgus v6- | Cm v6-peptide | Bachem #4142466 | 43 amino acids, peptide |
| peptide | | | synthesized, N-terminal biotin |
| Rabbit v6-peptide | Rb v6-peptide | Bachem #4148522 | 43 amino acids, peptide |
| | | | synthesized, N-terminal biotin |

### Example 1, Phage Display Selection on Human CD444v6 Using a Human scFv Library

Phage display selections were performed to enable isolation of scFv fragments with specificity for human CD44v6.

### Phage Display Selection

Biopanning was performed using four rounds of enrichment employing a naive human synthetic scFv phage library, SciLifeLib2 (SciLifeLab, Stockholm, Sweden), similar in design and construction to previously reported (Säll et al., Protein Eng Des Sel (2016) 29:427-437). In brief, human germline genes IGHV3-23 and IGKV1-39 were used as library scaffold and Kunkel mutagenesis was employed to introduce diversity into four of the six CDRs, namely VHCDR1, VHCDR2, VHCDR3 and VLCDR3. The selection was performed using chemically biotinylated human CD44v6, referred to as bio-CD44v6, and streptavidin-coated magnetic beads (Dynabeads M-280, ThermoFisher Scientific, #11206D). The selection pressure was increased by gradually decreasing the antigen amount (200 nM to 10 nM) and by increasing the intensity and number of washes between the different rounds. To remove non-specific or streptavidin binders, and to increase the likelihood of selecting v6-specific binders, pre-selection was performed by incubating the phage stock on streptavidin-beads with biotinylated human CD44 (isoform 6), referred to as bio-CD44, prior to selection round 1, 2 and 3. See table 10 for specifications of various antigens used. Also, 2% bovine serum albumin (BSA) was included as blocking agent throughout the selection procedure. Antigen-bound phages were eluted using a trypsin-aprotinin approach. The entire selection process was automated and performed with a Kingfisher Flex robot. Recovered phages were propagated in XL1 blue E. coli, either on agar plates at 37° C. overnight (Round 1) or in solution at 30° C. overnight (Rounds 2, 3 and 4). Phage stocks were made by infecting with an excess of M13K07 helper phage (New England Biolabs, #N0315S) and scFv expression induced by the addition of IPTG. The overnight cultures were PEG/NaCl-precipitated, resuspended in selection buffer and used for the next round of selection.

Re-Cloning and Expression of scFv

To allow production of soluble scFv fragments, phagemid DNA from selection rounds 3 and 4 was isolated. In pools, the genes encoding the scFv fragments were restriction enzyme digested and sub-cloned into a screening vector that provided a signal for secretion of scFv fragments along with a triple-FLAG tag and a hexahistidine (His) tag at the C-terminal. The constructs were subsequently transformed into TOP10 E. coli. Single colonies were picked, cultivated and IPTG-induced for soluble scFv expression in 96-well format. In total, 189 scFv clones present in bacterial supernatant were prepared for ELISA screen.

### ELISA Screen

CD44v6 and two negative control proteins, CD44 and streptavidin, were immobilized into a 384-well ELISA plate, either directly or indirectly through streptavidin at a concentration of 1 μg/ml. ScFv clones present in bacterial supernatant were diluted 1:10 in block buffer (PBS supplemented with 0.5% BSA+0.05% Tween20) and allowed to bind to the coated proteins. Detection of binding was enabled through an HRP-conjugated a-FLAG M2 antibody (Sigma-Aldrich #A8592) followed by incubation with TMB-ELSA substrate (ThermoFisher Scientific #34029). The colorimetric signal development was stopped by adding 1 M sulphuric acid and plates were analyzed at wavelength 450 nm. All samples were assayed in duplicates.

### DNA Sequencing

95 scFv clones showing binding to CD44v6 and/or CD44 were sent for Sanger DNA sequencing by Eurofins/GATC Biotech (Cologne, Germany).

### Results

A phage display selection was carried out on antigen human CD44v6 using scFv phage library SciLifeLib 2. Following re-cloning of selected scFv fragments into a screening vector, a total of 189 scFv clones were picked from selection round 3 and 4. ELISA screen resulted in 95 potential scFv positive hits. DNA sequencing of these hits resulted in the identification of 17 sequence unique scFv clones.

### Example 2, Kinetic Screen of 17 Sequence Unique scFvs by SPR

A kinetic screen-based approach by surface plasmon resonance (SPR) was performed on the 17 sequence unique scFv clones from Example 1 to enable ranking of the different clones.

### Materials and Methods

A kinetic screen was performed on a Biacore T200 instrument (Cytiva). An a-FLAG M2 antibody (Sigma-Aldrich #F1804), functioning as a capture ligand, was immobilized onto all four surfaces of a CM5-S amine sensor chip according to manufacturer's recommendations. The scFv clones present in bacterial supernatant were injected and captured onto the chip surfaces, followed by injection of 50 nM CD44v6 or 50 nM CD44 (negative control). The surfaces were regenerated with 10 mM glycin-HCl pH 2.1. All experiments were performed at 25° C. in running buffer (HBS supplemented with 0.05% Tween20, pH 7.5). By subtracting the response curve of a reference surface (an a-FLAG M2 antibody immobilized surface) and to a blank run (running buffer injected instead of antigen) response curve sensorgrams were obtained. Data was analyzed using the Biacore T200 Evaluation 3.1 software and a 1:1 Langmuir binding model.

### Results

Eleven scFv clones out of the 17 clones analyzed were considered promising based on binding to CD44v6. More specifically, a high binding response and favorable slow off-rate were considered.

### Example 3, Conversion to Full Antibody Format

11 scFv clones that specifically seemed to bind the v6 region of CD44v6 were selected for conversion to full-length human IgG4 S228P (EU numbering) antibody format. The rational for including these particular clones were based on performance in a panel of binding assays (see Examples 1 and 2). In addition, positive control BIWA-4 and isotype control antibodies were similarly converted.

In-Fusion Cloning, Transfection into HEK293, Expression and Purification

The VH and VL region of the selected scFv clones were PCR amplified and inserted into in house constructed vector pHAT-hIgG4-S241P using the In-Fusion HD Plus Cloning Kit (Clontech #638909). Transfection of plasmid DNA into expiHEK293 cells was performed using an ExpiFectamine™ 293 TransfectionKit (ThermoFisher Scientific #A 14525) in 4 ml cultures. The cultures were harvested after 5 days of cultivation (37° C., 6% CO2, 80% rH, 400 rpm) and the antibodies were purified on Protein A conjugated magnetic beads (ThermoFisher Scientific #88846) using a Kingfisher Flex instrument. Buffer was exchange to PBS, pH 7.5, using a 96-well spin desalting plate (ThermoFisher Scientific #87775). SDS-PAGE was performed to determine purity and integrity of the purified antibodies and concentrations were determined using an Implen NP80 UV-Vis Spectrophotometer.

### ELISA

CD44v6, human (hm) v6-peptide and negative controls CD44 and streptavidin, were immobilized into a 384-well ELISA plate, either directly or indirectly through streptavidin at a concentration of 1 μg/ml. Purified IgG4 S228P clones were diluted to 1, 0.2 or 0.04 g/ml in block buffer (PBS supplemented with 0.5% BSA+0.05% Tween20) and allowed to bind to the coated proteins. Detection of binding was enabled through an HRP-conjugated α-human kappa antibody (Southern Biotech #9230), followed by incubation with TMB-ELISA substrate (ThermoFisher Scientific #34029). The colorimetric signal development was stopped by adding 1 M sulphuric acid and plates were analyzed at wavelength 450 nm. All samples were assayed in duplicates.

### Results

All 11 scFv clones and BIWA-4 were successfully re-cloned to human IgG4 S228P format, expressed in expiHEK293 cells and purified by Protein A-conjugated magnetic beads on a Kingfisher Flex instrument. All antibodies were of the expected molecular weight with acceptable level of purity, as analyzed by SDS-PAGE, and ELISA confirmed retained binding to the v6-region of CD44v6, i.e. positive signals were obtained for CD44v6 and hm v6-peptide, whereas no binding was detected on CD44 and streptavidin. No binding was detected for isotype controls.

### Example 4, Kinetic Measurements of Novel Anti-CD44v6 IgG4 Antibodies

The kinetic constants of purified IgG4 clones (Example 3) to CD44 and v6-peptide were determined by surface plasmon resonance (SPR) using a single cycle kinetic (SCK) approach.

### Materials and Methods

Kinetic measurements were performed on a BIAcore T200 instrument (Cytiva) using a SCK approach. An α-human Kappa antibody (GE Healthcare #28958325), functioning as a capture ligand, was immobilized onto all four surfaces of a CM5-S amine sensor chip according to manufacturer's recommendations. Antibodies were injected and captured at equal response units (RU). A four-fold dilution series of CD44v6, consisting of five concentrations ranging between 80 nM-0.3 nM, were prepared in running buffer and sequentially injected over the chip surfaces. Single injections of 100 nM CD44 (negative control) was also performed.

For kinetic measurements of human v6-peptide, a streptavidin (SA) sensor chip was used to immobilize the v6-peptide at approximately 20 RU. A five-fold dilution series of each antibody, consisting of five concentrations ranging between 50 nM-0.08 nM, were prepared in running buffer and sequentially injected over the chip surfaces.

All experiments were performed at 25° C. in running buffer (HBS supplemented with 0.05% Tween20, pH 7.5) and the chip surfaces were regenerated with 10 mM glycine-HCl, pH 2.1. By subtracting the response curve of a reference surface (an α-human Kappa antibody immobilized surface or a streptavidin immobilized surface) and to a blank run (running buffer injected instead of antigen or antibody) response curve sensorgrams were obtained. Data was analyzed using the Biacore T200 Evaluation 3.1 software and a 1:1 Langmuir binding model.

### Results

Converted U-MN114-19 displayed retained binding towards CD44v6 and hm v6-peptide. Reference antibody BIWA-4 hIgG4 also displayed binding, as expected, towards CD44v6 and hm v6-peptide. Apparent affinities (denoted as appKD) were in the low to sub-nanomolar range. Compared to reference BIWA4 hIgG4, U-MN114-19 binds with higher affinity to both CD44v6 and hm v6-peptide, Table 11.

TABLE 11
Measured kinetic parameters, ka (M − 1 s − 1), kd (s − 1), K_{D} (M)
U-MN114-19 IgG4 and BIWA-4 IgG4 towards CD44v6 and hm v6-peptide.

| | CD44v6 | | | Human v6-peptide | | |
|---|---|---|---|---|---|---|
| Clone name | ka (1/Ms) | kd (1/s) | ^{app}K_{D} | ka (1/Ms) | kd (1/s) | ^{app}K_{D} |
| U-MN114-19 IgG4 | 3.2E+05 | 1.0E−03 | 3.3E−09 | 7.3E+05 | 1.8E−04 | 2.8E−10 |
| S228P | | | | | | |
| BIWA-4 IgG4 S228P | 6.5E+03 | 3.1E−04 | 4.8E−08 | 1.9E+05 | 1.7E−04 | 8.8E−10 |

### Example 5, Cell Binding of IgG4 U-MN114-19

All clones successfully converted and produced as IgG4 were assessed for cell binding following radiolabeling. For simplicity, data from U-MN114-19 and BIWA4 will be presented in this example.

### Materials and Methods

Radioiodination was performed with Pierce Iodination tubes according to manufacturer's protocol. In short, 2-5 MBq of 125| (PerkinElmer) was added to washed (1 mL PBS) Pierce Iodination tubes (ThermoFisher) containing 50 μL of PBS. The iodine was incubated in the tubes for 6 min with gentle swirling every 30 s before transferal to Eppendorf tubes containing 10 μg of MN114-antibody (hIgG4). The antibody/iodine reaction was incubated at 37° C. and 350 rpm for 15 min. Labeling yields were determined with ITLC. For ¹⁷⁷Lu-labeling, U-MN114-19 and BIWA-4 were first conjugated with DOTA using p-SCN-Bn-DOTA: A buffer switch from PBS to Na₂HPO₄ (0.1 M, pH 7.5-7.9, metal-free H₂0) was performed using an Amicon® Ultra 0.5 mL centrifugal filter (Sigma) according to manufacturer's instructions. A ten-fold molar excess of p-SCN-Bn-DOTA, dissolved in Na₂HPO₄ (0.1 M, ph 7.5-7.9, metal-free H₂0), was added to the antibodies and incubated for 4 h at 37° C. and 350 rpm. Following incubation, the antibodies were purified from excess p-SCN-Bn-DOTA using the Amicon® Ultra filters. Radiolabeling with ¹⁷⁷Lu (Curium Pharma) was performed by adding 5-10 MBq of ¹⁷⁷Lu to typically 50 μg of antibody and incubating for 2 h at 42° C. at 350 rpm. Labeling yields were determined by ITLC.

All LigandTracer experiments were carried out according to manufacturer's standard protocols. In short, 3-5*10⁵ BHT-101 or ACT-1 cells were seeded in a 10 cm petri dish and incubated at 37° C. and 5% CO₂ at least 24 h prior to the start of experiments. The dishes were placed in LigandTracer instruments (grey or yellow) and the baseline was established for approximately 30 min prior to adding the first concentration. Each concentration (1 nM and 3 nM or 1 nM, 3 nM and 10 nM for U-MN114-19 and 1 nM, 3 nM and 10 nM for BIWA-4) was run for approximately 90 min at RT. All medium containing radioactive antibodies was removed and the retention was started in 3 mL of fresh medium. For kinetic measurements, the total experimental run time was 12 h.

### Results

U-MN114-19 demonstrated low affinity binding to CD44v6-positive cell lines with varying antigen expression levels following labeling with both ¹²⁵I and ¹⁷⁷Lu, a representative example of iodinated antibodies on BHT-101 cells is shown in FIG. 7. On all cell lines, U-MN114-19 demonstrated lower or equal Kp compared to that of BIWA-4. On high-antigen expressing cell lines, the differences were marginal but on low/medium antigen expressing cell lines (e.g. BHT-101), U-MN119-14 was superior (FIG. 7).

### Example 6, Biodistribution Comparison Between U-MN114-19 and BIWA-4

The primary candidate from the U-MN114 antibody selection (U-MN114-19) was evaluated in vivo in balb/c nu/nu mice in a direct comparison with BIWA-4.

### Materials and Methods

Animal studies were performed according to Swedish Laws and regulations using ethical permits C33/16, C9/16 and 10966/20. For inoculation of tumor cells (ACT-1), following cell harvesting with trypsin, approximately 10⁷ cells per mouse were injected in 100 μL of serum-free medium in the right posterior flank of the mice. Radiolabeling was performed as described in Example 5. Radiolabeled U-MN114-19 or BIWA4 (both IgG4 formats) was injected intravenously (i.v.) in the tail vein 8-10 days post inoculation with tumor cells. A total of 15 μg of U-MN114-19 or BIWA4 was injected, consisting of 1-2 μg of radiolabeled antibody and 13-14 μg of non-radiolabeled antibody for a total of 15 μg/50 μL per mouse. The injected activity was between 100-300 kBq per mouse. Animals were euthanized and dissected at 24 h, 48 h, and 192 h (8 d) post injection (p.i.). The organs were analyzed in a Wizard1460 well-counter (PerkinElmer) and % ID was calculated for organ weight (g).

### Results

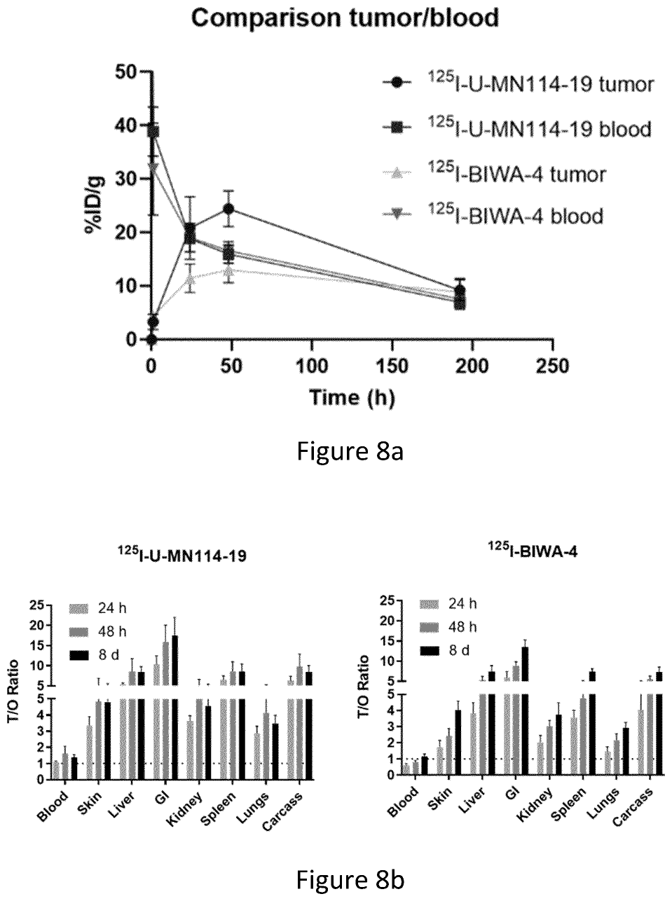

Biodistribution with ¹²⁵I-U-MN114-19 was evaluated repeatedly with reproducible results. In a in direct comparison with ¹²⁵I-BIWA-4 (FIGS. 8a and 8b), the peak tumor uptake of ¹²⁵I-U-MN114-19 was significantly higher than that of ¹²⁵I-BIWA4, and the total area under curve in tumors was significantly greater for ¹²⁵I-U-MN114-19 compared to ¹²⁵I-BIWA4.

### Conclusion

¹²⁵I-U-MN114-19 demonstrated superior tumor-to-blood ratios and greater peak tumor uptake than ¹²⁵I-BIWA4 in ACT-1 tumor bearing mice. The biodistribution results did not reveal any off-target binding or accumulation of the antibody, indicating a stable and specific compound. Results suggest a greater therapeutic utility with U-MN114-19 over BIWA4.

### Example 7, Dual-Nuclide Biodistribution of U-MN114-19

A dual-nuclide study using ¹²⁵I and ¹⁷⁷Lu was used to determine whether the antibody would be more effective in terms of tumor targeting, total tumor dose and safety with a halogen or radiometal. The data was later utilized for a dosimetry calculation (data not shown).

### Materials and Methods

Animal studies were carried out as described in Example 6, using the ACT-1 xenograft model. Radiolabeling was performed as in Example 5. A total of 15 μg antibody was injected per mouse, consisting of 1 μg of ¹²⁵I-U-MN114-19 (100 kBq) and 1 μg of ¹⁷⁷Lu-U-MN114-19 (100 kBq) in the same injection, diluted with 13 μg of unlabeled U-MN114-19. Animals were euthanized and dissected at 1 h, 24 h, 48 h, and 168 h post injection (p.i.). The organs were analyzed in a Wizard1460 well-counter (PerkinElmer) and % ID was calculated for organ weight (g).

### Results

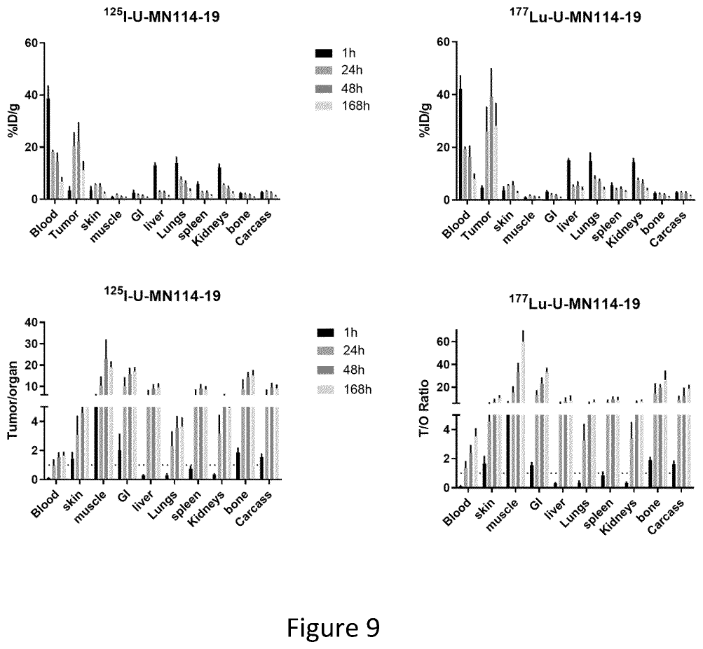

Peak tumor uptake of ¹⁷⁷Lu-U-MN114-19 was significantly greater than of ¹²⁵I-U-MN114-19 at all time points, resulting in superior tumor-to-blood ratios (FIG. 9). The dosimetry assessment based on the biodistribution data verified that ¹⁷⁷Lu would be a more suitable therapeutic nuclide moving forward. FIG. 9 shows; Top left: Biodistribution of ¹²⁵I-U-MN114-19 (IgG4), Bottom left: Tumor-to-organ ratios from biodistribution of ¹⁷⁷Lu-U-MN114-19 (IgG4) in ACT-1 xenografts. Top right: Biodistribution of ¹⁷⁷Lu-U-MN114-19 (IgG4), Bottom right: Tumor-to-organ ratios of ¹⁷⁷Lu-U-MN114-19 (IgG4). Error bars represent SD, N=15.

### Conclusion

¹⁷⁷Lu-U-MN114-19 was superior to ¹²⁵I-U-MN114-19 regarding peak tumor uptake and tumor-to-blood ratios. The dual-nuclide study suggested that ¹⁷⁷Lu would likely be the most effective therapeutic radionuclide in future studies due to the significantly greater tumor uptake and lower cross-fire dose to healthy tissues compared to ¹³¹I.

### Example 8, Affinity Maturation of U-MN114-19

Maturation of clone U-MN114-19 for the purpose of generating clones with improved affinity towards the v6-region within CD44v6.

### Library Design and Construction

Since U-MN114-19 originally originated in an scFv library (Example 1), the scFv format was chosen as scaffold for library generation. The affinity maturation library, denoted MN114-19-Lib1 (Lib1), were diversified in four of the six CDR-loops; VHCDR1, VHCDR2, VLCDR1 and VLCDR2. The CDRH3 of VH and VL are generally considered the most important regions for antigen binding. It was reasoned that these loops are likely also important for the target interaction of U-MN114-19 and were therefore kept constant. Next generation sequencing of natural repertoires has revealed that antibody evolution through somatic hypermutation occur through defined paths based on the germline gene origin of the antibody (DOI: 10.3389/fimmu.2018.01391). The spatial and chemical diversity introduced into the Lib1 was inspired by these in vivo evolution patterns. The 13 most substituted positions in VHCDR1 and VHCDR2 of natural IGHV3-23 repertoires were targeted for mutation and, analogously, the 5 most mutated positions in VLCDR1 and VLCDR2 of IGKV1-39 were targeted. Also, amino acids composition at each of these positions were motivated by the natural repertoires. Altogether, the procedure resulted in targeting of 18 positions in MN114-19 Lib1, creating a combinatorial theoretical diversity of approximately 3.2×10⁸ variants.

The library diversity was introduced into the scaffold genes using Kunkel mutagenesis basically as described (Fellouse F A, Sidhu, S. S. (2007)). To assess whether the intended diversity had been incorporated, TOP10 E. coli cells were chemically transformed with a small aliquot of the generated Kunkel DNA and 96 clones were picked and sent for sequencing (GATC, Germany). The remaining DNA was subsequently electroporated into SS320 cells (Lucigen, Middleton, WI, USA), yielding highly diverse libraries containing approximately 1.5×10⁹ clones, as measured by the number of bacterial colonies obtained after transformation. The transformed SS320 cells were harvested and stored with 15% glycerol at −80° C. The bacterial glycerol stock was used to inoculate a total of 600 ml 2×YT with antibiotics selective for both the phagemid and the F′ episome. The bacteria were grown until exponential phase and then infected by M13KO7 helper phages (New England Biolabs, Ipswich, MA, USA) using a multiple of infection of five. The cultures were propagated overnight and scFv displaying phages were harvested by standard polyethylene glycol (PEG)/NaCl precipitation.

### Phage Display Selection

Phage display selection was performed using three or four rounds of enrichment employing MN114-19 Lib1. For biotinylated targets (CD44v6 and hm v6-peptide), the selection was performed using streptavidin-coated magnetic beads (as described in Example 1). Analogously, protein G-coated magnetic beads (Thermofisher Scientific #10004D) were used to capture Fc-fused CD44v6 (non-biotinylated). The selection tracks were designed so that only CD44v6 or hm v6-peptide were used throughout the selection rounds, or both antigens were alternated between different selection rounds, resulting in a scheme covering a total of four distinct selection tracks. The selection pressure was increased between the different selection rounds by decreasing the antigen amount (50-1 or 0.05 nM) and by increasing the intensity and number of washes (5-8). A negative selection (pre-selection) prior to round 1 was performed using Bio-CD44. Elution of antigen-bound phages was performed using a trypsin-aprotonin approach. The entire phage display selection process, except the phage-target antigen incubation step, was automated and performed with a Kingfisher Flex robot (ThermoFisher Scientific).

Re-Cloning and Expression of AL-MN114 scFv Clones

Phagemid DNA from selection round 2 and 3, or 2, 3 and 4, of each selection track was isolated, enzyme digested and sub-cloned in pool into in-house screening vector pHAT-6 to enable soluble expression of AL-MN114 scFv clones in fusion with a triple FLAG-tag and a hexahistidine tag (His×6) at the C-terminal. Vector constructs were subsequently transformed into E. coli TOP10 cells. Single colony clones were picked, cultivated and IPTG-induced for soluble scFv expression in 96-well format. In total, 468 scFv clones were prepared to be assayed in a primary ELISA screen.

### ELISA Screen

Antigens Bio-CD44v6 and hm v6-peptide, together with negative control Bio-CD44, were in-directly coated through streptavidin into a 384-ELISA well plate at 1 μg/ml in PBS. 3×FLAG-tagged scFv clones, present in bacterial supernatant, were diluted 1:7 in block buffer (PBS 0.5% BSA+0.05% Tween20) and allowed to bind to coated antigens. Detection of binding was enabled through an HRP-conjugated a-FLAG M2 antibody (Sigma-Aldrich #A8592) or an HRP-conjugated α-human KAPPA antibody (Southern biotech #2060-05) followed by incubation with TMB ELISA substrate (ThermoFisher Scientific #34029). The colorimetric-signal development was stopped by adding 1 M sulfuric acid and plates were analyzed at wavelength 450 mm (abs 450 nm) on a Spectramax plus instrument (Molecular Devices). All clones were assayed in duplicates from which a mean Abs 450 nm-value was calculated, and background subtracted with the mean Abs 450 nm-value of a blank sample (block buffer added instead of a scFv clone).

### DNA Sequencing

Clones displaying binding towards CD44v6 and hm v6-peptide were sent for Sanger DNA sequencing to Eurofins genomics (Ebersberg, Germany).

### Results

A total of 18 positions were targeted in the gene encoding the U-MN114-19 scFv in order to create MN114-Lib1. Sequencing of 96 randomly picked clones confirmed the introduction of the intended diversity.

Following phage display selections and ELISA screen of the 468 scFv clones, 354 positive hits were identified and only showing binding towards the v6-region within CD44v6 and not towards negative control antigen CD44. DNA sequencing of the 354 positive hits resulted in the identification of 247 sequence unique clones. These 247 clones were denoted AL-MN114, followed by a unique numerical number.

### Example 9, Kinetic Measurements of New Selected AL-MN114 scFv Clones

The 247 sequence unique AL-MN114 scFv clones (Example 8) were further analyzed by SPR in an initial kinetic screen for binding to hm v6-peptide. The most promising 95 clones, ranked on apparent off-rates, were further subjected to more fine kinetic measurements using a SCK approach towards CD44v6, human v6-peptide and cynomolgus v6-peptide.

### Materials and Methods

A kinetic screen and SCK measurements were performed on a BIAcore T200 instrument (Cytiva). An a-FLAG M2 antibody (Merck #F1804), functioning as a capture ligand, was immobilized onto all 4 surfaces of a CM5 series S sensor chip using EDC/NHS amine coupling chemistry according to manufacturer's recommendations. All experiments were performed at 25° C. in running buffer (HBS supplemented with 0.05% Tween20, pH 7.5) 3×FLAG-tagged AL-MN114 scFv clones present in bacterial supernatant were diluted in running buffer to obtain equal capture RU levels. For the kinetic screen, 100 nM hm v6-peptide (Table 10, Example 13) was prepared in running buffer and injected over an AL-MN114 scFv-captured surface and allowed to bind. For SCK measurements, a 3-fold dilution series of hm v6-peptide and cm v6-peptide (Table 10, Example 13), comprised of five concentrations ranging between 200-2.5 nM, were prepared in running buffer. Each dilution series was sequentially injected, starting from lowest to highest antigen concentration. All chip surfaces were regenerated with 10 mM glycin-HCl, pH 2.1.

By subtracting the response curve of a reference surface (an a-FLAG M2 antibody immobilized surface) and to a blank run (running buffer injected instead of antigen) response curve sensorgrams were obtained. Data was analyzed using the Biacore T200 Evaluation 3.1 software and a 1:1 Langmuir binding model.

### Results

The SCK measurements of clones correlated well with the apparent affinities obtained during kinetic screen. The most promising clones displayed an almost 5-fold improvement in off-rates compared to the parental clone U-MN114-19. The most promising twenty AL-MN114 scFv clones were converted into hIgG4 format as previously described (Example 3) and further characterized.

### Example 10, Characterization of AL-MN114 hIgG4 Clones on Cancer Cells

This experiment describes time-resolved interaction analysis (LigandTracer) and kinetic properties of the assessed radiolabeled U-MN114-19, AL-MN114 antibodies and BIWA4.

### Materials and Methods

Radiolabeling, LigandTracer cell seeding and experiments were carried out as described in Example 5.

### Results

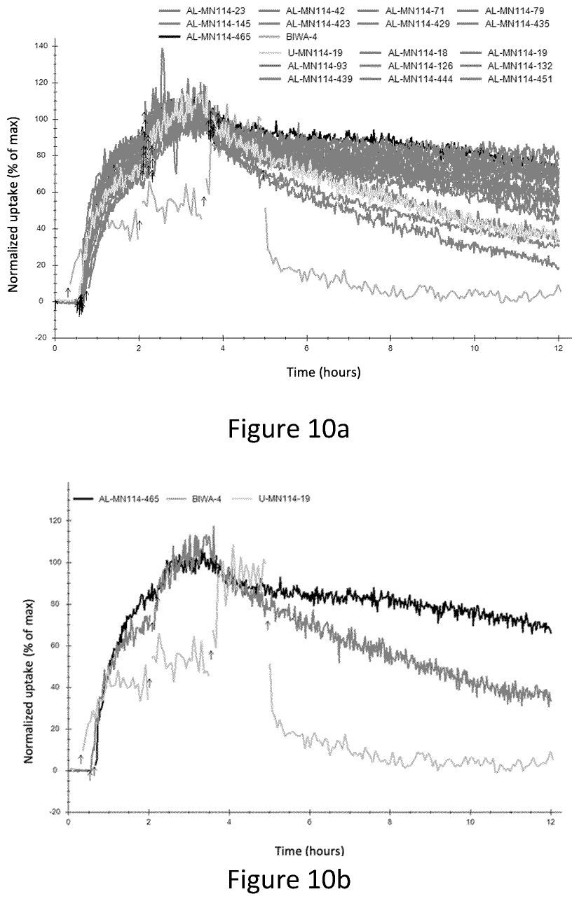

The anaplastic thyroid cancer cell line BHT-101 was used for LigandTracer evaluation of the affinity-matured AL-MN114-antibodies (hIgG4). The cell line was chosen based on its CD44v6-antigen level (medium), in order to better detect differences in affinity. All ¹²⁵I-MN114-antibodies were superior to ¹²⁵I-BIWA4 in affinity (FIG. 10). The majority of ¹²⁵I-AL-MN114 clones demonstrated a superior retention than the parental clone, ¹²⁵I-U-MN114-19 (Table 12). As for the ¹⁷⁷Lu-labeled antibodies measured on LigandTracer on A431 cells (high antigen expressing cell line), both U-MN114-19 and AL-MN114-465 were superior to BIWA4 in affinity (Table 13). Additionally, both ¹²⁵I/¹⁷⁷Lu-AL-MN114-465 had a superior retention compared to BIWA4 and U-MN114-19.

TABLE 12
Kinetic evaluation of LigandTracer data from ¹²⁵I-labeled
MN114 antibodies and BIWA-4 on BHT-101 cells.

| Antibody | kₐ | k_{d} | K_{D} | Model |
|---|---|---|---|---|
| ¹²⁵I-U-MN114-19 | 2*10⁵ | 3*10⁻⁵ | 2*10⁻¹⁰ | 1:1 |
| ¹²⁵I-AL-MN114-18 | 5*10⁵ | 1*10⁻⁵ | 2*10⁻¹¹ | 1:1 |
| ¹²⁵I-AL-MN114-19 | 3*10⁵ | 1*10⁻⁵ | 4*10⁻¹¹ | 1:1 |
| ¹²⁵I-AL-MN114-23 | 4*10⁵ | 2*10⁻⁵ | 4*10⁻¹¹ | 1:1 |
| ¹²⁵I-AL-MN114-42 | 5*10⁵ | 2*10⁻⁵ | 4*10⁻¹¹ | 1:1 |
| ¹²⁵I-AL-MN114-71 | 4*10⁵ | 8*10⁻⁶ | 2*10⁻¹¹ | 1:1 |
| ¹²⁵I-AL-MN114-79 | 3*10⁵ | 9*10⁻⁶ | 3*10⁻¹¹ | 1:1 |
| ¹²⁵I-AL-MN114-93 | 4*10⁵ | 5*10⁻⁵ | 1*10⁻¹⁰ | 1:1 |
| ¹²⁵I-AL-MN114-126 | 2*10⁵ | 8*10⁻⁶ | 4*10⁻¹¹ | 1:1 |
| ¹²⁵I-AL-MN114-132 | 4*10⁵ | 1*10⁻⁵ | 4*10⁻¹¹ | 1:1 |
| ¹²⁵I-AL-MN114-145 | 3*10⁵ | 4*10⁻⁵ | 1*10⁻¹⁰ | 1:1 |
| ¹²⁵I-AL-MN114-423 | 3*10⁵ | 3*10⁻⁵ | 1*10⁻¹⁰ | 1:1 |
| ¹²⁵I-AL-MN114-429 | 3*10⁵ | 2*10⁻⁵ | 6*10⁻¹¹ | 1:1 |
| ¹²⁵I-AL-MN114-435 | 5*10⁵ | 9*10⁻⁶ | 2*10⁻¹¹ | 1:1 |
| ¹²⁵I-AL-MN114-439 | 3*10⁵ | 8*10⁻⁶ | 2*10⁻¹¹ | 1:1 |
| ¹²⁵I-AL-MN114-444 | 2*10⁵ | 4*10⁻⁶ | 2*10⁻¹¹ | 1:1 |
| ¹²⁵I-AL-MN114-451 | 3*10⁵ | 9*10⁻⁶ | 3*10⁻¹¹ | 1:1 |
| ¹²⁵I-AL-MN114-465 | 4*10⁵ | 1*10⁻⁵ | 3*10⁻¹¹ | 1:1 |
| ¹²⁵I-BIWA4* | 6*10⁵ | 6*10⁻⁴ | 9*10⁻⁹ | 1:1 |

*BIWA4 affinity calculated from three added concentrations as opposed to two (1 nM, 3 nM and 10 nM).

TABLE 13
Kinetic evaluation and comparison of LigandTracer data
of ¹⁷⁷Lu-labeled U-MN114-19, AL-MN114-465 and BIWA4
on A431 cells (high antigen expressing cell line).

| Antibody | kₐ | k_{d} | K_{D} | Model |
|---|---|---|---|---|
| ¹⁷⁷Lu-BIWA4 | 3*10⁴ | 10*10⁻⁶ | 3*10⁻¹⁰ | 1:1 |
| ¹⁷⁷Lu-AL-MN114- | 2*10⁵ | 5*10⁻⁶ | 3*10⁻¹¹ | 1:1 |
| 465 | | | | |
| ¹⁷⁷Lu-U-MN114-19 | 1*10⁵ | 1*10⁻⁵ | 9*10⁻¹¹ | 1:1 |

### Conclusion

Affinity maturation resulted in improved affinities, surpassing that of the parental clone as well as the comparator antibody BIWA4. Four AL-MN114 clones were chosen for hIgG1 conversion and small-scale production: AL-MN114-71, AL-MN114-132, AL-MN114-444 and AL-MN114-465.

### Example 11, Conversion to Full Antibody IgG1 Format

AL-MN114-71, AL-MN114-132, AL-MN114-444, AL-MN114-465, U-MN114-19 and BIWA4 were converted to human IgG1 LALA and/or human IgG1 LALA IAHA formats, see Table 14. For simplicity, the conversion into IgG1 LALA format is described below.

InFusion Cloning, Transfection into HEK293, Expression and Purification

The VH and VL region of AL-MN114-132, AL-MN114-465 and BIWA4 were PCR amplified and inserted into in house constructed vector pHAT-hIgG1-LALA using the In-Fusion HD Plus Cloning Kit (Clontech #638909). Transfection of plasmid DNA into expiHEK293 cells was performed using an ExpiFectamine™ 293 TransfectionKit (ThermoFisher Scientific #A14525) in 230 mL cultures. The cultures were harvested after 5 days of cultivation (37° C., 7% CO2, 70% rH, 105 rpm) and the antibodies were purified by affinity chromatography using a HiTrap PrismA column (Cytiva) followed by buffer exchange to PBS, pH 7.4, using on a HiTrap desalting column. Endotoxin levels were <0.25 EU/mg as determined by LALA chromogenic endotoxin assay. SDS-PAGE was performed to determine purity and integrity of the purified antibodies and concentrations were determined using an Implen NP80 UV-Vis Spectrophotometer. In addition, size exclusion chromatography was run on each of the purified antibody on an Agilent Bio SEC-3.

TABLE 14
Clones converted into full format
IgG1 LALA and/or IgG1 LALA IAHA.

| Clone name | IgG1 Format |
|---|---|
| U-MN114-19 | IgG1 LALA, IgG LALA IAHA |
| AL-MN114-71 | IgG1 LALA IAHA |
| AL-MN114-132 | IgG1 LALA, IgG LALA IAHA |
| AILMN114-444 | IgG1 LALA IAHA |
| AL-MN114-465 | IgG1 LALA, IgG LALA IAHA |
| BIWA4 | IgG1 LALA, IgG LALA IAHA |

### Results

Selected clones were successfully converted into IgG1 LALA and/or IgG1 LALA IAHA formats, produced and purified.

### Example 12, Epitope Mapping

Antibody clones from Example 12 were epitope mapped within the human v6-region by ELISA, using a peptide-based approach. A peptide array comprised of 29 synthesized peptides, covering the 43 amino acids long human v6-region were ordered from JPT peptides (Germany). Each peptide was 15 amino acids long with 1 amino acid shift carrying a biotin moiety at the N-terminal.

### Materials and Methods

The peptide array and the full length human v6-peptide (43 aa) were coated into a 384-ELISA well plates through streptavidin (1 μg/mL). Purified antibodies (Example 11) were all diluted to 1 μg/mL in block buffer (PBS supplemented with 0.5% BSA+0.05% Tween20) and allowed to bind to coated peptides. Detection of binding was enabled through an HRP-conjugated α-human kappa antibody (Southern Biotech #9230), followed by incubation with TMB-ELISA substrate (ThermoFisher Scientific #34029). The colorimetric signal development was stopped by adding 1 M sulphuric acid and plates were analyzed at wavelength 450 nm. Each sample was assayed in duplicates from which a mean absorbance value was calculated and background subtraction with the mean absorbance value of a blank well (block buffer added instead of antibody).

### Results

Results showed that all MN114 clones displayed the same six amino acid long epitope, amino acids WFGNRW (SEQ ID NO: 7) located at position 18-23 within the human v6-regions. BIWA-4 displayed a ten amino acid long partly overlapping epitope, amino acids WFGNRWHEGY (SEQ ID NO: 5) located at position 18-27 within the human v6-regions.

### Conclusion

The assessed MN114 antibody clones share an identical six amino acid long epitope, partly overlapping with the ten amino acid long epitope of BIWA-4.

### Example 13, Kinetic Measurements of IgG1 Clones

Kinetic measurements following conversion of AL-MN114-71, -132, -444, -465 and BIWA4, into the IgG1 format was performed by SPR using a SCK approach. The parental clone U-MN114-19 as IgG1 was also included.

### Materials and Methods

The SPR measurements were performed on a BIAcore T200 instrument (Cytiva) using a SCK approach. Each antibody was directly immobilized onto a separate surface of a CM5 series S sensor chip using EDC/NHS amine coupling chemistry according to manufacturer's recommendations. Immobilization levels were set to 1500 RU. All experiments were performed at 25° C. in running buffer (HBS, 0.05% Tween20, pH 7.5). A three-fold dilution series of CD44v6 and negative control CD44, comprised of five concentrations ranging between 10-0.12 nM, were prepared in running buffer. A four-fold dilution series of human, cynomolgus and rabbit v6-peptide, respectively, comprised of five concentrations ranging between 100-0.39 nM, were also prepared in running buffer. Each dilution series were sequentially injected, starting with the lowest concentration first, over an immobilized antibody clone and chip surfaces were regenerated with 10 mM glycin-HCl, pH 2.1. See table 10 for specifications of antigens used in SPR measurements. By subtracting the response curve of a reference surface (an activated and deactivated chip surface) and to a blank run (running buffer injected instead of antigen) response curve sensorgrams were obtained. Data was analyzed using the Biacore T200 Evaluation 3.1 software and a 1:1 Langmuir binding model.

### Results

All antibody clones demonstrating binding towards CD44v6 but not towards CD44 (control antigen). The obtained apparent affinities, appKD, towards CD44v6 were in the sub-nanomolar range, ^{app}K_{D}=1-2 nM, Table 15. Binding of antibody clones were also detected towards human (hm) v6-peptide, cynomolgus (cm) v6-peptide and rabbit (rb) v6-peptide, in contrast to BIWA4, which did not demonstrate binding to rb-v6-peptide. This data correlates well with performed epitope mapping (Example 12), which demonstrated that all novel antibodies share a six amino acid long epitope present in cynomolgus and rabbit v6-region. The epitope found for BIWA4 is not present in rabbit v6-region, only in cynomolgus v6-region. Introduced LALA or LALA IAHA Fc-alterations are expected to have no impact on the antigen-binding part of an antibody.

TABLE 15
Measured kinetic parameters, ka (1/Ms), kd (1/s),
K_{D} (M) or ^{app}K_{D} (M) of IgG1 antibodies towards CD44v6,
CD44, hm v6-peptide, cm v6-peptide and rb v6-peptide.

| | CD44v6 | | |
|---|---|---|---|
| Clone name | ka (1/Ms) | kd (1/s) | ^{app}K_{D}(M) |
| U-MN114-19 IgG1 LALA | 1.3E+05 | 1.5E−04 | 1.2E−09 |
| U-MN114-19 IgG1 LALA IAHA | 2.5E+06 | 1.2E−04 | 5.0E−11 |
| AL-MN114-71 IgG1 LALA IAHA | 2.3E+05 | 2.6E−04 | 1.1E−09 |
| AL-MN114-132 IgG1 LALA | 1.9E+06 | 1.2E−04 | 6.2E−11 |
| AL-MN114-132 IgG1 LALA IAHA | 1.4E+05 | 2.1E−04 | 1.5E−09 |
| AL-MN114-444 IgG1 LALA IAHA | 3.2E+05 | 3.2E−04 | 1.0E−09 |
| AL-MN114-465 IgG1 LALA | 1.3E+06 | 1.0E−04 | 7.7E−11 |
| AL-MN114-465 IgG1 LALA IAHA | 1.7E+05 | 2.7E−04 | 1.6E−09 |
| BIWA4 IgG1 LALA | 3.7E+05 | 2.7E−04 | 7.2E−10 |
| BIWA4 IgG1 LALA IAHA | 2.4E+04 | 3.3E−04 | 1.4E−08 |
| Clone name | ka (1/Ms) | kd (1/s) | K_{D}(M) |
| | Human v6-peptide | | |
| U-MN114-19 IgG1 LALA | 8.3E+05 | 4.9E−02 | 5.8E−08 |
| U-MN114-19 IgG1 LALA IAHA | 1.1E+06 | 1.9E−02 | 1.7E−08 |
| AL-MN114-71 IgG1 LALA IAHA | 6.2E+05 | 1.1E−02 | 1.7E−08 |
| AL-MN114-132 IgG1 LALA | 1.1E+06 | 1.3E−02 | 1.2E−08 |
| AL-MN114-132 IgG1 LALA IAHA | 7.3E+05 | 1.6E−02 | 2.2E−08 |
| AL-MN114-444 IgG1 LALA IAHA | 7.1E+05 | 8.3E−03 | 1.2E−08 |
| AL-MN114-465 IgG1 LALA | 9.3E+05 | 8.0E−03 | 8.6E−09 |
| AL-MN114-465 IgG1 LALA IAHA | 6.8E+05 | 9.3E−03 | 1.4E−08 |
| BIWA4 IgG1 LALA | 2.4E+05 | 3.5E−03 | 1.5E−08 |
| BIWA4 IgG1 LALA IAHA | 3.6E+04 | 2.7E−03 | 7.3E−08 |
| | Cynomolgus v6-peptide | | |
| U-MN114-19 IgG1 LALA | 1.0E+06 | 4.6E−02 | 4.4E−08 |
| U-MN114-19 IgG1 LALA IAHA | 1.7E+06 | 3.4E−02 | 2.0E−08 |
| AL-MN114-71 IgG1 LALA IAHA | 7.1E+05 | 9.5E−03 | 1.3E−08 |
| AL-MN114-132 IgG1 LALA | 1.3E+06 | 9.6E−03 | 7.3E−09 |
| AL-MN114-132 IgG1 LALA IAHA | 8.6E+05 | 1.4E−02 | 1.7E−08 |
| AL-MN114-444 IgG1 LALA IAHA | 7.2E+05 | 7.9E−03 | 1.1E−08 |
| AL-MN114-465 IgG1 LALA | 9.9E+05 | 6.2E−03 | 6.2E−09 |
| AL-MN114-465 IgG1 LALA IAHA | 6.8E+05 | 8.5E−03 | 1.3E−08 |
| BIWA4 IgG1 LALA | 2.8E+05 | 3.5E−03 | 1.2E−08 |
| BIWA4 IgG1 LALA IAHA | 8.3E+04 | 3.6E−03 | 4.4E−08 |
| | Rabbit v6-peptide | | |
| U-MN114-19 IgG1 LALA | 2.0E+06 | 1.5E−01 | 7.9E−08 |
| U-MN114-19 IgG1 LALA IAHA | 2.2E+06 | 6.7E−02 | 3.1E−08 |
| AL-MN114-71 IgG1 LALA IAHA | 7.1E+05 | 1.6E−02 | 2.2E−08 |
| AL-MN114-132 IgG1 LALA | 1.7E+06 | 2.3E−02 | 1.3E−08 |
| AL-MN114-132 IgG1 LALA IAHA | 9.9E+05 | 3.7E−02 | 3.7E−08 |
| AL-MN114-444 IgG1 LALA IAHA | 8.2E+05 | 1.2E−02 | 1.5E−08 |
| AL-MN114-465 IgG1 LALA | 1.2E+06 | 1.0E−02 | 8.2E−09 |
| AL-MN114-465 IgG1 LALA IAHA | 8.3E+05 | 1.5E−02 | 1.9E−08 |
| BIWA4 IgG1 LALA | — | — | — |
| BIWA4 IgG1 LALA IAHA | — | — | — |

| | CD44 | | |
|---|---|---|---|
| Clone name | ka (1/Ms) | kd (1/s) | ^{app}K_{D}(M) |
| U-MN114-19 IgG1 LALA | — | — | — |
| U-MN114-19 IgG1 LALA IAHA | — | — | — |
| AL-MN114-71 IgG1 LALA IAHA | — | — | — |
| AL-MN114-132 IgG1 LALA | — | — | — |
| AL-MN114-132 IgG1 LALA IAHA | — | — | — |
| AL-MN114-444 IgG1 LALA IAHA | — | — | — |
| AL-MN114-465 IgG1 LALA | — | — | — |
| AL-MN114-465 IgG1 LALA IAHA | — | — | — |
| BIWA4 IgG1 LALA | — | — | — |
| BIWA4 IgG1 LALA IAHA | — | — | — |

— No binding detected

### Conclusion

AL-MN114-71, -132, -444, -465 and parental clone U-MN114-19 displayed binding towards hm v6-peptide, cm v6-peptide, rb v6-peptide and CD44v6 iso4.

### Example 14, Specificity Following Radiolabeling

The specificity of radiolabeled MN114-antibodies, as evaluated by a specificity assay where radiolabeled antibodies compete for antigen binding with a molar excess of unlabeled antibodies, was assessed using two ATC cell lines.

### Materials and Methods

Radioiodination with ¹²⁵I and radiolabeling with ¹⁷⁷Lu was performed as described in Example 5. ACT-1 and BHT-101 (3-5*10⁴ cells per well) were seeded in 48-well plates at least 24 h prior to the start of the experiments and incubated at 37° C. and 5% CO2. 30 nM of radiolabeled antibodies or 30 nM of radiolabeled antibody in solution with 3 UM of excess unlabeled antibody, were added per well (100 μL) and incubated at 37° C. and 5% CcO₂ for 24 h. After incubation, cells were washed with PBS three times and harvested using 100 μL of trypsin per well. Cells were counted and CPM, as measured in a Wizard1460 well counter (PerkinElmer), were adjusted for cell count, resulting in data presented as CPM/100000 cells. LigandTracer dishes were seeded as described in Example 7. For the competition assay, 10 nM of ¹²⁵I-U-MN114-19 and ¹²⁵I-AL-MN114-444 was incubated for 2 h before adding 30 nM of unlabeled antibody.

### Results

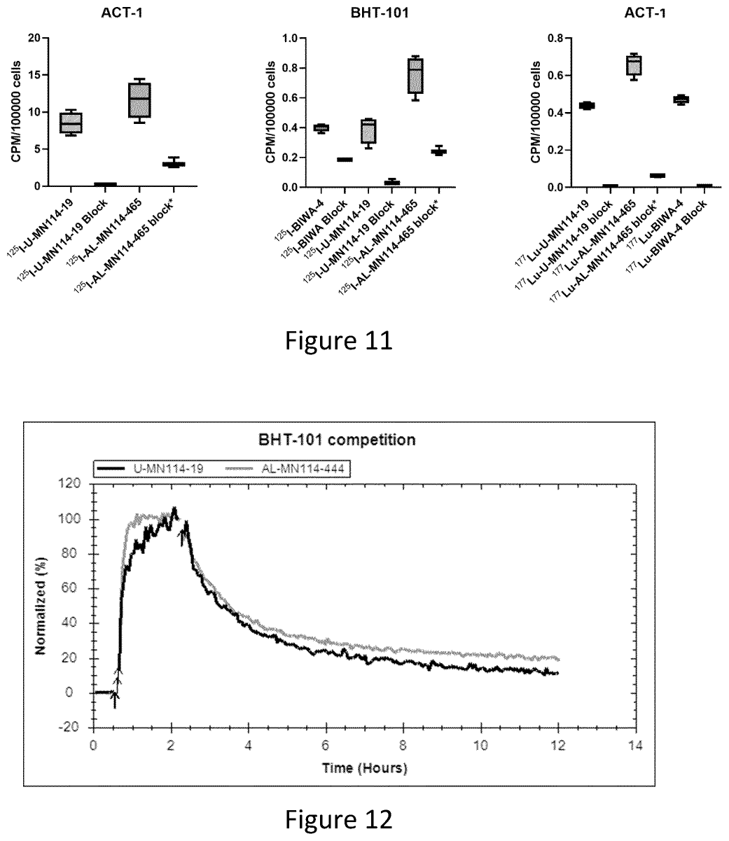

All radiolabeled antibodies retained specificity following labeling with both ¹²⁵I and ¹⁷⁷Lu (FIG. 11) on both ACT-1 and BHT-101 cells, where specificity of AL-MN114-465 was performed in the presence of 100-fold molar excess of the parental antibody, U-MN114-19. The total CPM/100000 cells was greater for AL-MN114-465 than for both BIWA4 and U-MN114-19, indicating greater uptake on both cell lines of AL-MN114-465, regardless of radiolabeling method. Similarly, the affinity-matured AL-MN114-444 had a slower dissociation rate than U-MN114-19, once again indicating the superior retention and affinity of the affinity-matured clones (FIG. 12).

### Conclusion

Both U-MN114-19 and affinity matured variants (AI-MN114-465) bind with greater specificity and superior affinity than BIWA4 and in an antigen-dependent manner. Additionally, AL-MN114-444 had an improved retention in the presence of a three-fold molar excess of unlabeled antibody than U-MN114-19.

### Example 15, Biodistribution of IgG1 LALA and LALA/IAHA Formats in Mice

Four selected AL-MN114 clones were assessed in vivo in tumor xenografts both as LALA and LALA/IAHA formats with both ¹²⁵I and ¹⁷⁷Lu.

### Material and Methods

Animal studies were carried out as described in Example 6, using the ACT-1 or A431 xenograft model. Radiolabeling was performed as in Example 5. For the ¹²⁵I-comparison of LALA versus LALA/IAHA, a total of 15 μg was injected per mouse, consisting of 1 μg of ¹²⁵I-U-MN114-19 (100 kBq) 14 μg of unlabeled U-MN114-19 (IgG1 LALA or IgG1 LALA/IAHA). Animals were euthanized and dissected at 1 h, 24 h, 48 h, and 168 h p.i. (IgG1 LALA) and 1 h, 4 h, 24 h, 48 h, 72 h, 96 h and 168 h p.i. (IgG1 LALA/IAHA). The organs were analyzed in a Wizard1460 well-counter (PerkinElmer) and % ID was calculated for organ weight (g). For dual nuclide studies of U-MN114-19 and AL-MN114-71, 132, 444 and 465, a total of 15 μg was injected per mouse, consisting of 1 μg of ¹²⁵I-U-MN114-19 (100 kBq) and 1 μg of ¹⁷⁷Lu-U-MN114-19 (100 kBq) in the same injection, diluted with 13 μg of unlabeled U-MN114-19. For ¹⁷⁷Lu-labeled IgG1 LALA antibodies, 1 μg of labeled antibody and 14 μg of unlabeled antibody was injected per animal in 50 μL. Animals were euthanized and dissected at 24 h, 48 h, 96 h and 168 h post injection (p.i.). The organs were analyzed in a Wizard1460 well-counter (PerkinElmer) and % ID was calculated for organ weight (g).

### Results

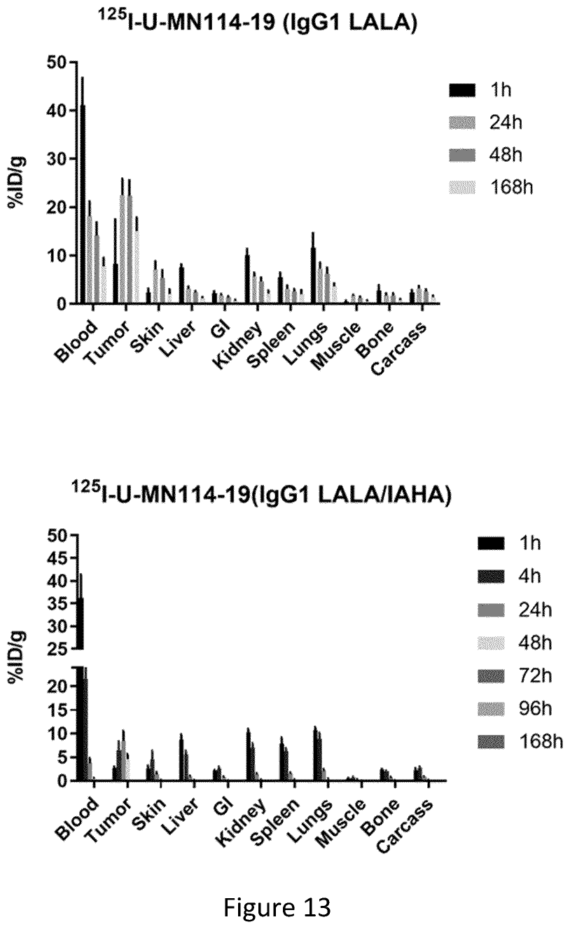

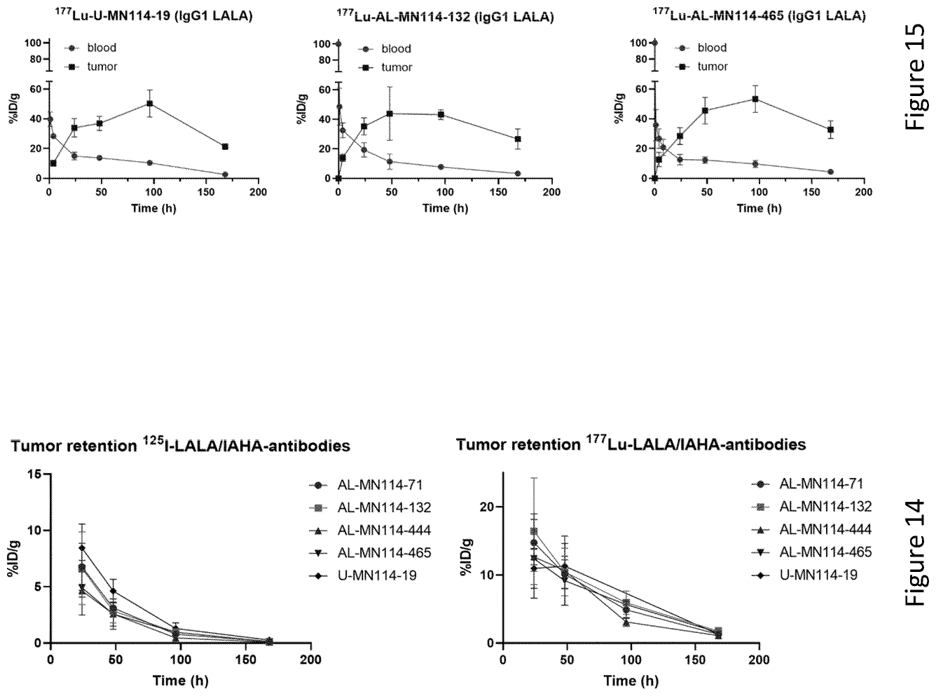

The IgG1 LALA/IAHA format demonstrated higher tumor-to-blood ratios compared to the IgG1 LALA format, whereas the absolute tumor dose was higher with the LALA formats (FIG. 13). The affinity matured IgG1 clones evaluated in vivo and U-MN114-19 all displayed good tumor uptake and retention. ¹⁷⁷Lu-labeled antibodies had a higher peak tumor uptake and improved retention compared to the ¹²⁵I-labeled antibodies (FIG. 14).

Two AL-MN114-clones were evaluated as IgG1 LALA in vivo using ¹⁷⁷Lu and compared to U-MN114-19 IgG1 LALA, all displaying favorable biodistribution profiles, with higher tumor uptake compared to blood. The affinity matured variants displayed a better tumor to blood ratio compared to the parental version (FIG. 15).

### Conclusion

The assessed affinity matured IgG1 clones all displayed good tumor uptake and retention in vivo. ¹⁷⁷Lu-labeled antibodies demonstrated improved tumor uptake and retention compared to ¹²⁵I-labeled antibodies.

### Example 16, Therapy Studies in Mice

Two separate studies on two different xenograft models, ACT-1 and BHT-101, evaluated the therapeutic ability of U-MN114-19 labeled with ¹⁷⁷Lu.

### Material and Methods

Animal studies were carried out as described in Example 6, using the ACT-1 and BHT-101 xenograft models. The xenograft models were chosen based on their antigen expression levels. ACT-1 has an estimated 10-fold greater expression level of CD44v6 than that of BHT-101. For ACT-1 xenografts, radiolabeling was performed as in Example 5, with the exception of injected activity of ¹⁷⁷Lu and amount of antibody injected per antibody. For therapy studies, approximately 15 MBq of ¹⁷⁷Lu-U-MN114-19 (50 μg, IgG4) was injected per animal. In control animals, 50 μg of unlabeled U-MN114-19 (IgG4) was injected. For BHT-101 xenografts, 10⁷ cells were inoculated in the right posterior flank as described in Example 6. For therapy of BHT-101 tumors, approximately 7 MBq of ¹⁷⁷Lu-U-MN114-19 (50 μg, IgG4) was injected per animal. For controls, approximately 7 MBq of ¹⁷⁷Lu-isotope control ab (IgG4) was injected per animal. Tumors were measured and tumor volume calculated through (H×L×W)*0.52. Animal weights were monitored for general health.

### Results

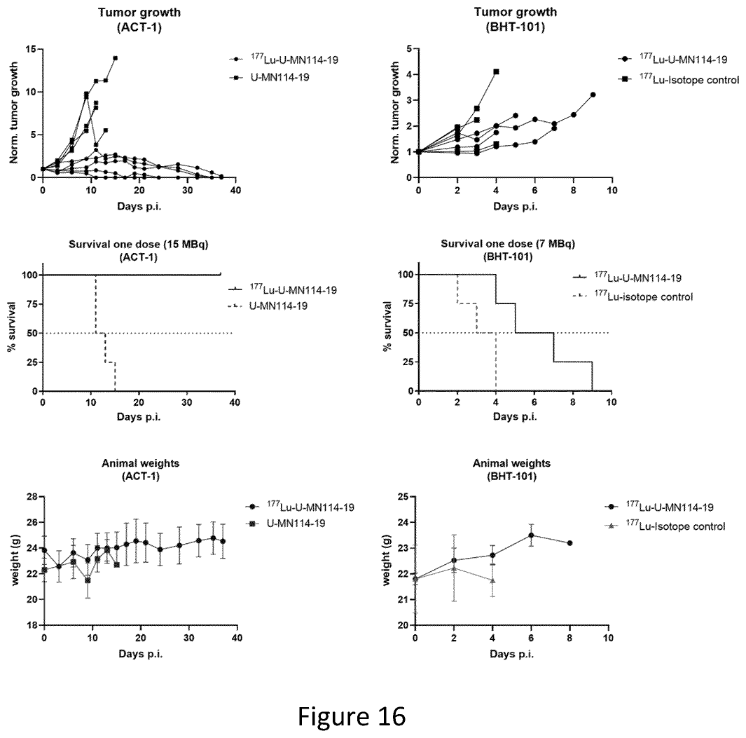

In the ACT-1 study, the single dose (15 MBq) resulted in complete remission in all treated animals, with no signs of tumor regrowth by the end of the study. In the BHT-101 study, the 7 MBq-dose resulted in delay of tumor growth compared to the isotope control, nearly doubling median tumor survival (FIG. 16).

### Conclusion

The therapy studies demonstrate the therapeutic potential in both high-(ACT-1) and medium (BHT-101) tumor models.

### Example 17, Comparisons with BIWA4

To establish the efficiency of the current antibodies compared to the prior art, a series of experiments was conducted. For example, the affinity and treatment efficiency were tested.

### A. Affinity

Regarding the affinity, the approximate dissociation constant K_{D} (^{app}K_{D}) was evaluated, where the K_{D} value could be seen as relating to the concentration of antibody (the amount of antibody needed for a particular experiment), and so the lower the K_{D} value (lower concentration), thus the higher the affinity of the antibody.

It was established that the parental clone MN19 (also referred to as U-MN114-19) has an approximate Kp, ^{app}K_{D}, of approximately 0.16 nM on BHT-101 cells (i.e. cells of the BHT-101 cell line, DSMZ no. ACC 279, originating from thyroid carcinoma). All affinity matured variants of the parental MN19 (as defined in tables 3-6) show an appKD below 0.2 nM as measured on BHT-101. On the other hand, BIWA4 has an appKD on the BHT-101 cell line of 9 nM, which is significantly higher. It may be noted in this regard that, to be able to compare, the BIWA4 tested in these experiments are performed using a BIWA4 antibody in the identical format/construct of the antibodies of the present disclosure. On SPR, the affinities are higher than on the live cells.

### B. Biodistribution

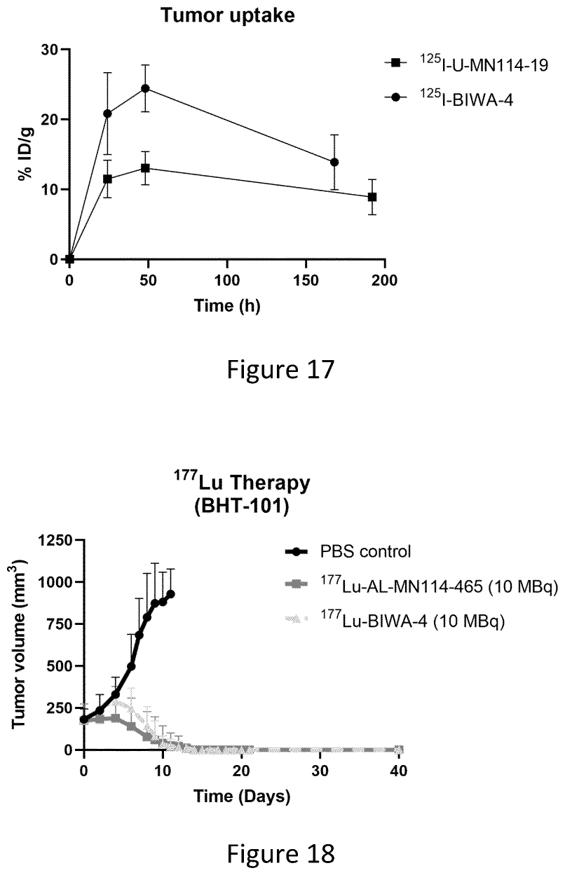

Regarding treatment efficiency using the antibodies, biodistribution of the injected dose (ID) was determined as % ID/g for the conjugated antibodies. Biodistribution of the iodinated parental clone U-MN114-19, ¹²⁵I-U-MN114-19 and iodinated BIWA4, ¹²⁵I-BIWA4, revealed significantly superior tumor uptake of ¹²⁵I-U-MN114-19 compared to ¹²⁵I-BIWA4, as illustrated in FIG. 17. FIG. 17 shows a plot of comparison of tumor uptake of ¹²⁵I-U-MN114-19 and ¹²⁵I-BIWA4 in ACT-1 xenografts. The calculated area under the curve, AUC, assuming 0% of the injected activity is in the tumors at t=0, was significantly greater for ¹²⁵I-U-MN114-19 than ¹²⁵I-BIWA4

### C. Response Times and Effect on Tumor Growth

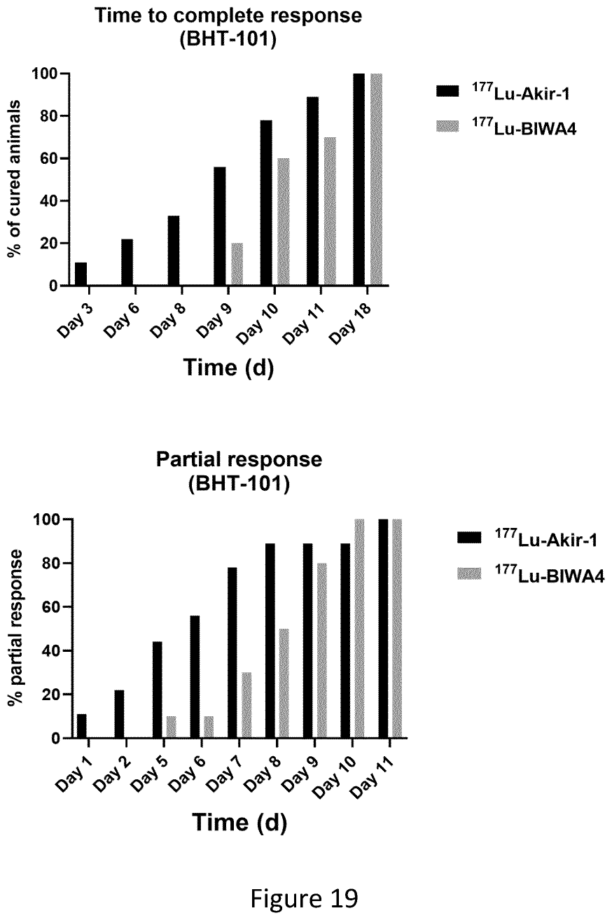

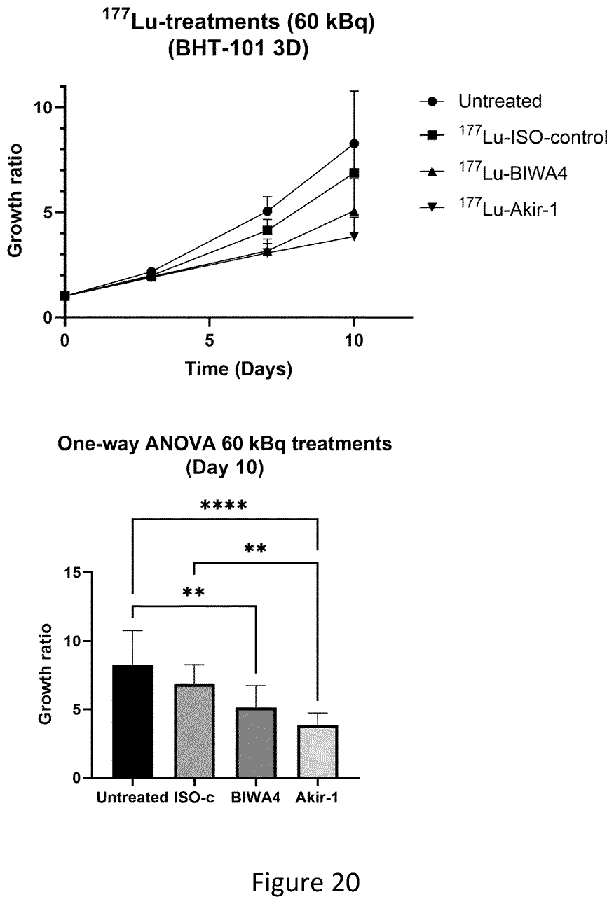

In an animal study using xenograft-bearing mice, ¹⁷⁷Lu-BIWA4 and ¹⁷⁷Lu-AL-MN114-465 were tested side-by-side in BHT-101 xenografts. A single dose (10 MBq/50 μg) was injected in the tail vein of the mice and caliper measurements followed the growth of the xenografts, while animal weights and behavior were used to monitor animal health. The results are shown in FIGS. 18 and 19.

FIG. 18 illustrates tumor growth following treatment with 10 MBq of ¹⁷⁷Lu-AL-MN114-465 or ¹⁷⁷Lu-BIWA4 in BHT-101 xenografts. It may be seen that survival was 100% for animals treated with either ¹⁷⁷Lu-AL-MN114-465 and ¹⁷⁷Lu-BIWA4, whereas all controls were euthanized by day 12 p.i. FIG. 19 illustrates time to complete response, where FIG. 19 (top) shows time to complete response of ¹⁷⁷Lu-AL-MN114-465 or ¹⁷⁷Lu-BIWA4 in BHT-101 xenografts, and FIG. 19 (bottom) shows time to partial response of ¹⁷⁷Lu-AL-MN114-465 or ¹⁷⁷Lu-BIWA4 in BHT-101 xenografts. It may be seen that both complete and partial responses were faster for animals treated with ¹⁷⁷Lu-AL-MN114-465 compared to ¹⁷⁷Lu-BIWA4 in the BHT-101 xenografts.

In a 3D multicellular tumor spheroid assay using BHT-101 cells, treatment with 60 kBq of ¹⁷⁷Lu-AL-MN114-465 resulted in significantly smaller spheroids at day 10 post treatment compared both untreated controls and spheroids treated with 60 kBq of ¹⁷⁷Lu-Isotope control antibody. Similarly, treatment with 60 kBq ¹⁷⁷Lu-BIWA4 resulted in significantly smaller spheroids compared to untreated controls at day 10 post treatment, but not compared to spheroids treated with 60 kBq ¹⁷⁷Lu-Isotope control antibody. Thus, while treatment with both 60 kBq of ¹⁷⁷Lu-AL-MN114-465 and ¹⁷⁷Lu-BIWA4 had a significant effect on spheroid growth compared to untreated controls, BIWA4 failed to have a significantly greater impact than the isotope control. This indicates that AL-MN114-465 is superior to BIWA4 in this 3D setting (FIG. 20).

FIG. 20 illustrates the size/growth ratios of the tumors, where FIG. 20 (top) shows growth ratios of 3D multicellular tumor spheroids of BHT-101 cells treated with 60 kBq of either ¹⁷⁷Lu-AL-MN114-465, BIWA4 or an isotope control (ISO-c) antibody. The spheroids are measured over time and the growth ratio is defined as relative size compared to size at day 0 (start of treatment). FIG. 20 (bottom) shows a one-way ANOVA of the size ratios at Day 10 post treatment, which demonstrated that while spheroids treated with AL-MN114-465 did not differ significantly from those treated with BIWA4, they did differ (**) from the isotope control, which spheroids treated with BIWA4 failed to do.

### Conclusions

All the binding proteins of the present disclosure as defined in Tables 3-6, the parental clone and the maturated clones, show higher affinity compared to BIWA4. Typically, the dissociation constant K_{D} as measured on cells from the BHT-101 cell line is below 9 nM, such as below 8, 7, 6, 5, 4, 3, 2, or 1 nM, preferably below 1 nM, such as below 0.8, 0.7, 0.6, 0.5, 0.4, 0.3 or 0.2 nM. Preferably, the CD44v6-binding proteins binds to BHT-101 cells such that the KD value of the interaction is at most 0.2 nM, such that K_{D}<0.2 nM for the disclosed binding proteins.

Further, it may be seen that the binding proteins of the current disclosure give rise to faster response in treatment, and greater treatment effects. Thus, the present binding proteins have a significant technical effect compared to the prior art BIWA4 antibody.

### ITEMIZED EMBODIMENTS

1. A binding protein that specifically binds CD44v6 and comprises a binding domain of an antibody, the binding domain comprising a heavy chain variable domain (VH) and a light chain variable domain (VL) or derivatives thereof, each VL and VH comprising three complementarity determining regions (CDRs), wherein the amino acid sequences of the CDRs are selected from the group comprising:
VHCDR1 as defined by SEQ ID NO: 1;
VHCDR2 as defined by SEQ ID NO: 2;
VHCDR3 as defined by SEQ ID NO: 3;
VLCDR1 as defined by SEQ ID NO: 4;
VLCDR2 as defined by X₁AS, where X₁ may be T, A, or S;
VLCDR3 as defined by SEQ ID NO: 6;
and CDR sequences having 95% or more, such as 96%, 97%, 98%, 99% or more, identity thereto, wherein said binding protein recognises an epitope of CD44v6 as defined by SEQ ID NO: 7.

2. The binding protein of item 1, wherein the VHCDR1 is defined by the amino acid sequence GFX₃FX₅X₆X₇A, and wherein X₃ is S, X₅ is G, X₆ is S and/or X₇ is Y.

3. The binding protein of items 1 or 2, wherein the VHCDR2 is defined by the amino acid sequence ISX₃X₄GX₆ST and wherein X₃ is A, X₄ is G and/or X₆ is S.

4. The binding protein of items 1-3, wherein the VLCDR1 is defined by the amino acid sequence QX₂₁X₄X₅Y and wherein X₂ is S, X₄ is S and/or X₅ is S.

5. The binding protein of items 1-4, wherein the VLCDR2 is defined by the amino acid sequence X₁AS and wherein X₁ is T or S.

6. The binding protein of item 1, wherein VHCDR1, VHCDR2 and VLCDR2 are present next to specific framework amino acids, wherein the CDR and framework amino acid (faa) sequences are selected from the group comprising:
VHCDR1 and faa defined by SEQ ID NO: 8;
VHCDR2 and faa as defined by SEQ ID NO: 9;
VLCDR2 and faa as defined by SEQ ID NO: 10;
and CDR sequences having 95% or more, such as 96%, 97%, 98%, 99% or more, identity thereto.

7. The binding protein of items 1-5, wherein the CDRs are individually selected from the group comprising:
VHCDR1 is selected from SEQ ID NO: 11-18;
VHCDR2 is selected from SEQ ID NO: 19-25, 32;
VHCDR3 as defined by SEQ ID NO: 3;
VLCDR1 is selected from SEQ ID NO: 26-31;
VLCDR2 is selected from TAS, SAS and AAS;
VLCDR3 as defined by SEQ ID NO:6;
and CDR sequences having 95% or more, such as 96%, 97%, 98%, 99% or more, identity thereto.

8. The binding protein of items 1-5 and 7, wherein the CDRs are selected from the group comprising:
VHCDR1 as defined by SEQ ID NO: 11;
VHCDR2 is selected from SEQ ID NO: 19-25;
VHCDR3 as defined by SEQ ID NO: 3;
VLCDR1 as defined by SEQ ID NO: 26;
VLCDR2 is selected from TAS, SAS and AAS;
VLCDR3 as defined by SEQ ID NO:6;
and CDR sequences having 95% or more, such as 96%, 97%, 98%, 99% or more, identity thereto.

9. The binding protein of items 1-5 and 7-8, wherein the amino acid sequences of the CDRs are selected from the group comprising:
i) a binding protein having
   VHCDR1 as defined by SEQ ID NO: 11;
   VHCDR2 as defined by SEQ ID NO: 19;
   VHCDR3 as defined by SEQ ID NO: 3;
   VLCDR1 as defined by SEQ ID NO: 26;
   VLCDR2 as defined by TAS; and
   VLCDR3 as defined by SEQ ID NO: 6;
ii) a binding protein having
   VHCDR1 as defined by SEQ ID NO: 11;
   VHCDR2 as defined by SEQ ID NO: 20;
   VHCDR3 as defined by SEQ ID NO: 3;
   VLCDR1 as defined by SEQ ID NO: 26;
   VLCDR2 as defined by SAS; and
   VLCDR3 as defined by SEQ ID NO: 6;
iii) a binding protein having
   VHCDR1 as defined by SEQ ID NO: 12;
   VHCDR2 as defined by SEQ ID NO: 20;
   VHCDR3 as defined by SEQ ID NO: 3;
   VLCDR1 as defined by SEQ ID NO: 26;
   VLCDR2 as defined by AAS; and
   VLCDR3 as defined by SEQ ID NO: 6;
iv) a binding protein having
   VHCDR1 as defined by SEQ ID NO: 16;
   VHCDR2 as defined by SEQ ID NO: 20;
   VHCDR3 as defined by SEQ ID NO: 3;
   VLCDR1 as defined by SEQ ID NO: 26;
   VLCDR2 as defined by AAS; and
   VLCDR3 as defined by SEQ ID NO: 6;
v) a binding protein having
   VHCDR1 as defined by SEQ ID NO: 17;
   VHCDR2 as defined by SEQ ID NO: 20;
   VHCDR3 as defined by SEQ ID NO: 3;
   VLCDR1 as defined by SEQ ID NO: 27;
   VLCDR2 as defined by SAS; and
   VLCDR3 as defined by SEQ ID NO: 6;
vi) a binding protein having
   VHCDR1 as defined by SEQ ID NO: 12;
   VHCDR2 as defined by SEQ ID NO: 21;
   VHCDR3 as defined by SEQ ID NO: 3;
   VLCDR1 as defined by SEQ ID NO: 26;
   VLCDR2 as defined by AAS; and
   VLCDR3 as defined by SEQ ID NO: 6;
vii) a binding protein having
   VHCDR1 as defined by SEQ ID NO: 12;
   VHCDR2 as defined by SEQ ID NO: 22;
   VHCDR3 as defined by SEQ ID NO: 3;
   VLCDR1 as defined by SEQ ID NO: 27;
   VLCDR2 as defined by TAS; and
   VLCDR3 as defined by SEQ ID NO: 6;
viii) a binding protein having
   VHCDR1 as defined by SEQ ID NO: 13;
   VHCDR2 as defined by SEQ ID NO: 19;
   VHCDR3 as defined by SEQ ID NO: 3;
   VLCDR1 as defined by SEQ ID NO: 27;
   VLCDR2 as defined by SAS; and
   VLCDR3 as defined by SEQ ID NO: 6;
ix) a binding protein having
   VHCDR1 as defined by SEQ ID NO: 11;
   VHCDR2 as defined by SEQ ID NO: 19;
   VHCDR3 as defined by SEQ ID NO: 3;
   VLCDR1 as defined by SEQ ID NO: 27;
   VLCDR2 as defined by SAS; and
   VLCDR3 as defined by SEQ ID NO: 6;
x) a binding protein having
   VHCDR1 as defined by SEQ ID NO: 14;
   VHCDR2 as defined by SEQ ID NO: 19;
   VHCDR3 as defined by SEQ ID NO: 3;
   VLCDR1 as defined by SEQ ID NO: 27;
   VLCDR2 as defined by SAS; and
   VLCDR3 as defined by SEQ ID NO: 6;
xi) a binding protein having
   VHCDR1 as defined by SEQ ID NO: 14;
   VHCDR2 as defined by SEQ ID NO: 19;
   VHCDR3 as defined by SEQ ID NO: 3;
   VLCDR1 as defined by SEQ ID NO: 26;
   VLCDR2 as defined by AAS; and
   VLCDR3 as defined by SEQ ID NO: 6;
xii) a binding protein having
   VHCDR1 as defined by SEQ ID NO: 12;
   VHCDR2 as defined by SEQ ID NO: 19;
   VHCDR3 as defined by SEQ ID NO: 3;
   VLCDR1 as defined by SEQ ID NO: 26;
   VLCDR2 as defined by AAS; and
   VLCDR3 as defined by SEQ ID NO: 6;
xiii) a binding protein having
   VHCDR1 as defined by SEQ ID NO: 15;
   VHCDR2 as defined by SEQ ID NO: 19;
   VHCDR3 as defined by SEQ ID NO: 3;
   VLCDR1 as defined by SEQ ID NO: 26;
   VLCDR2 as defined by AAS; and
   VLCDR3 as defined by SEQ ID NO: 6;
xiv) a binding protein having
   VHCDR1 as defined by SEQ ID NO: 17;
   VHCDR2 as defined by SEQ ID NO: 19;
   VHCDR3 as defined by SEQ ID NO: 3;
   VLCDR1 as defined by SEQ ID NO: 28;
   VLCDR2 as defined by AAS; and
   VLCDR3 as defined by SEQ ID NO: 6;
xv) a binding protein having
   VHCDR1 as defined by SEQ ID NO: 15;
   VHCDR2 as defined by SEQ ID NO: 19;
   VHCDR3 as defined by SEQ ID NO: 3;
   VLCDR1 as defined by SEQ ID NO: 26;
   VLCDR2 as defined by AAS; and
   VLCDR3 as defined by SEQ ID NO: 6;
xvi) a binding protein having
   VHCDR1 as defined by SEQ ID NO: 12;
   VHCDR2 as defined by SEQ ID NO: 19;
   VHCDR3 as defined by SEQ ID NO: 3;
   VLCDR1 as defined by SEQ ID NO: 26;
   VLCDR2 as defined by AAS; and
   VLCDR3 as defined by SEQ ID NO: 6;
xvii) a binding protein having
   VHCDR1 as defined by SEQ ID NO: 12;
   VHCDR2 as defined by SEQ ID NO: 23;
   VHCDR3 as defined by SEQ ID NO: 3;
   VLCDR1 as defined by SEQ ID NO: 29;
   VLCDR2 as defined by SAS; and
   VLCDR3 as defined by SEQ ID NO: 6;
xviii) a binding protein having
   VHCDR1 as defined by SEQ ID NO: 15;
   VHCDR2 as defined by SEQ ID NO: 20;
   VHCDR3 as defined by SEQ ID NO: 3;
   VLCDR1 as defined by SEQ ID NO: 30;
   VLCDR2 as defined by TAS; and
   VLCDR3 as defined by SEQ ID NO: 6;
xix) a binding protein having
   VHCDR1 as defined by SEQ ID NO: 18;
   VHCDR2 as defined by SEQ ID NO: 24;
   VHCDR3 as defined by SEQ ID NO: 3;
   VLCDR1 as defined by SEQ ID NO: 31;
   VLCDR2 as defined by SAS; and
   VLCDR3 as defined by SEQ ID NO: 6;
xx) a binding protein having
   VHCDR1 as defined by SEQ ID NO: 12;
   VHCDR2 as defined by SEQ ID NO: 25;
   VHCDR3 as defined by SEQ ID NO: 3;
   VLCDR1 as defined by SEQ ID NO: 26;
   VLCDR2 as defined by AAS; and
   VLCDR3 as defined by SEQ ID NO: 6;
xxi) a binding protein having
   VHCDR1 as defined by SEQ ID NO: 12;
   VHCDR2 as defined by SEQ ID NO: 32;
   VHCDR3 as defined by SEQ ID NO: 3;
   VLCDR1 as defined by SEQ ID NO: 26;
   VLCDR2 as defined by AAS; and
   VLCDR3 as defined by SEQ ID NO: 6;
and CDR sequences having 95% or more, such as 96%, 97%, 98%, 99% or more, identity thereto.

10. The binding protein of item 6, wherein the sequences of the CDRs including the framework amino acids (faa) are selected from the group comprising:
i) a binding protein having
   VHCDR1 and faa defined by SEQ ID NO: 121;
   VHCDR2 and faa as defined by SEQ ID NO: 129; and
   VLCDR2 and faa as defined by SEQ ID NO: 139;
ii) a binding protein having
   VHCDR1 and faa defined by SEQ ID NO: 121;
   VHCDR2 and faa as defined by SEQ ID NO: 130; and
   VLCDR2 and faa as defined by SEQ ID NO: 140;
iii) a binding protein having
   VHCDR1 and faa defined by SEQ ID NO: 122;
   VHCDR2 and faa as defined by SEQ ID NO: 131; and
   VLCDR2 and faa as defined by SEQ ID NO: 141;
iv) a binding protein having
   VHCDR1 and faa defined by SEQ ID NO: 126;
   VHCDR2 and faa as defined by SEQ ID NO: 130; and
   VLCDR2 and faa as defined by SEQ ID NO: 144;
v) a binding protein having
   VHCDR1 and faa defined by SEQ ID NO: 127;
   VHCDR2 and faa as defined by SEQ ID NO: 130; and
   VLCDR2 and faa as defined by SEQ ID NO: 140;
vi) a binding protein having
   VHCDR1 and faa defined by SEQ ID NO: 122;
   VHCDR2 and faa as defined by SEQ ID NO: 133; and
   VLCDR2 and faa as defined by SEQ ID NO: 141;
vii) a binding protein having
   VHCDR1 and faa defined by SEQ ID NO: 122;
   VHCDR2 and faa as defined by SEQ ID NO: 134; and
   VLCDR2 and faa as defined by SEQ ID NO: 142;
viii) a binding protein having
   VHCDR1 and faa defined by SEQ ID NO: 123;
   VHCDR2 and faa as defined by SEQ ID NO: 129; and
   VLCDR2 and faa as defined by SEQ ID NO: 140;
ix) a binding protein having
   VHCDR1 and faa defined by SEQ ID NO: 121;
   VHCDR2 and faa as defined by SEQ ID NO: 132; and
   VLCDR2 and faa as defined by SEQ ID NO: 140;
x) a binding protein having
   VHCDR1 and faa defined by SEQ ID NO: 124;
   VHCDR2 and faa as defined by SEQ ID NO: 129; and
   VLCDR2 and faa as defined by SEQ ID NO: 143;
xi) a binding protein having
   VHCDR1 and faa defined by SEQ ID NO: 124;
   VHCDR2 and faa as defined by SEQ ID NO: 132; and
   VLCDR2 and faa as defined by SEQ ID NO: 141;
xii) a binding protein having
   VHCDR1 and faa defined by SEQ ID NO: 122;
   VHCDR2 and faa as defined by SEQ ID NO: 129; and
   VLCDR2 and faa as defined by SEQ ID NO: 141;
xiii) a binding protein having
   VHCDR1 and faa defined by SEQ ID NO: 125;
   VHCDR2 and faa as defined by SEQ ID NO: 129; and
   VLCDR2 and faa as defined by SEQ ID NO: 141;
xiv) a binding protein having
   VHCDR1 and faa defined by SEQ ID NO: 127;
   VHCDR2 and faa as defined by SEQ ID NO: 135; and
   VLCDR2 and faa as defined by SEQ ID NO: 144;
xv) a binding protein having
   VHCDR1 and faa defined by SEQ ID NO: 125;
   VHCDR2 and faa as defined by SEQ ID NO: 132; and
   VLCDR2 and faa as defined by SEQ ID NO: 141;
xvi) a binding protein having
   VHCDR1 and faa defined by SEQ ID NO: 122;
   VHCDR2 and faa as defined by SEQ ID NO: 132; and
   VLCDR2 and faa as defined by SEQ ID NO: 141;
xvii) a binding protein having
   VHCDR1 and faa defined by SEQ ID NO: 122;
   VHCDR2 and faa as defined by SEQ ID NO: 136; and
   VLCDR2 and faa as defined by SEQ ID NO: 145;
xviii) a binding protein having
   VHCDR1 and faa defined by SEQ ID NO: 125;
   VHCDR2 and faa as defined by SEQ ID NO: 131; and
   VLCDR2 and faa as defined by SEQ ID NO: 146;
xix) a binding protein having
   VHCDR1 and faa defined by SEQ ID NO: 128;
   VHCDR2 and faa as defined by SEQ ID NO: 137; and
   VLCDR2 and faa as defined by SEQ ID NO: 145;
xx) a binding protein having
   VHCDR1 and faa defined by SEQ ID NO: 122;
   VHCDR2 and faa as defined by SEQ ID NO: 138; and
   VLCDR2 and faa as defined by SEQ ID NO: 141;
xxi) a binding protein having
   VHCDR1 and faa defined by SEQ ID NO: 122;
   VHCDR2 and faa as defined by SEQ ID NO: 33; and
   VLCDR2 and faa as defined by SEQ ID NO: 141;
and CDR sequences having 95% or more, such as 96%, 97%, 98%, 99% or more, identity thereto.

11. The binding protein of items 1-5 and 7-10 wherein the VH sequence comprises an amino acid sequence selected from the group consisting of SEQ ID NO: 35-54 and sequences having 80% or more, such as 85%, 90%, 95% or more, identity thereto, and wherein the VL sequence comprises an amino acid sequence selected from the group consisting of SEQ ID NO: 55-74 and sequences having 80% or more, such as 85%, 90%, 95% or more, identity thereto.

12. The binding protein of item 11, wherein the CDR sequences comprise no variations in the amino acid sequence, or wherein the sequence variation of the CDR amino acid sequences is at most 5%, such as 94%, 3%, 2%, 1% or less.

13. The binding protein of items 11-12, wherein the amino acid sequences of the VH and VL are selected from the group comprising:
i) a binding protein having
   VH as defined by SEQ ID NO: 35; and
   VL as defined by SEQ ID NO: 55;
ii) a binding protein having
   VH as defined by SEQ ID NO: 36; and
   VL as defined by SEQ ID NO: 56;
iii) a binding protein having
   VH as defined by SEQ ID NO: 37; and
   VL as defined by SEQ ID NO: 57;
iv) a binding protein having
   VH as defined by SEQ ID NO: 46; and
   VL as defined by SEQ ID NO: 66;
v) a binding protein having
   VH as defined by SEQ ID NO: 54; and
   VL as defined by SEQ ID NO: 74;
vi) a binding protein having
   VH as defined by SEQ ID NO: 38; and
   VL as defined by SEQ ID NO: 58;
vii) a binding protein having
   VH as defined by SEQ ID NO: 39; and
   VL as defined by SEQ ID NO: 59;
viii) a binding protein having
   VH as defined by SEQ ID NO: 40; and
   VL as defined by SEQ ID NO: 60;
ix) a binding protein having
   VH as defined by SEQ ID NO: 41; and
   VL as defined by SEQ ID NO: 61;
x) a binding protein having
   VH as defined by SEQ ID NO: 42; and
   VL as defined by SEQ ID NO: 62;
xi) a binding protein having
   VH as defined by SEQ ID NO: 43; and
   VL as defined by SEQ ID NO: 63;
xii) a binding protein having
   VH as defined by SEQ ID NO: 44; and
   VL as defined by SEQ ID NO: 64;
xiii) a binding protein having
   VH as defined by SEQ ID NO: 45; and
   VL as defined by SEQ ID NO: 65;
xiv) a binding protein having
   VH as defined by SEQ ID NO: 47; and
   VL as defined by SEQ ID NO: 67;
xv) a binding protein having
   VH as defined by SEQ ID NO: 48; and
   VL as defined by SEQ ID NO: 68;
xvi) a binding protein having
   VH as defined by SEQ ID NO: 49; and
   VL as defined by SEQ ID NO: 69;
xvii) a binding protein having
   VH as defined by SEQ ID NO: 50; and
   VL as defined by SEQ ID NO: 70;
xviii) a binding protein having
   VH as defined by SEQ ID NO: 51; and
   VL as defined by SEQ ID NO: 71;
xix) a binding protein having
   VH as defined by SEQ ID NO: 52; and
   VL as defined by SEQ ID NO: 72;
xx) a binding protein having
   VH as defined by SEQ ID NO: 53; and
   VL as defined by SEQ ID NO: 73;
xxi) a binding protein having
   VH as defined by SEQ ID NO: 147; and
   VL as defined by SEQ ID NO: 148;
and sequences having 80% or more, such as 85%, 90%, 95% or more, identity thereto.

14. The binding protein of items 1-13, wherein the binding protein is a monoclonal antibody, or an antigen-binding fragment selected from the group consisting of Fv fragments, Fab-like fragments and domain antibodies.

15. The binding protein of item 14, wherein the Fv fragment is an scFv fragment.

16. The binding protein of item 14, wherein the Fab-like fragment is a Fab or F(ab′)2 fragment.

17. The binding protein of items 1-14, wherein the binding molecule is a monoclonal antibody of the IgG1 isotype, such as an IgG1 LALA antibody or IgG1 LALA IAHA antibody, or a monoclonal antibody of the IgG4 isotype.

18. The binding protein of items 1-17, wherein the binding protein is human or of human origin.

19. The binding protein of items 17-18, wherein the antibody comprises a heavy chain and a light chain, the heavy chain comprising an amino acid sequence selected from the group consisting of SEQ ID NO: 34, 75-93 and 149, and sequences having 80% or more, such as 85%, 90%, 95% or more, identity thereto; and the light chain comprising an amino acid sequence selected from the group consisting of SEQ ID NO: 94-113 and 150, and sequences having 80% or more, such as 85%, 90%, 95% or more, identity thereto.

20. The binding protein of item 19, wherein the amino acid sequences of the heavy chain and the light chain are selected from the group comprising:
i) a binding protein having
   Heavy chain as defined by SEQ ID NO: 75; and
   Light chain as defined by SEQ ID NO: 94;
ii) a binding protein having
   Heavy chain as defined by SEQ ID NO: 76; and
   Light chain as defined by SEQ ID NO: 95;
iii) a binding protein having
   Heavy chain as defined by SEQ ID NO: 77; and
   Light chain as defined by SEQ ID NO: 96;
iv) a binding protein having
   Heavy chain as defined by SEQ ID NO: 34; and
   Light chain as defined by SEQ ID NO: 105;
v) a binding protein having
   Heavy chain as defined by SEQ ID NO: 93; and
   Light chain as defined by SEQ ID NO: 113;
vi) a binding protein having
   Heavy chain as defined by SEQ ID NO: 78; and
   Light chain as defined by SEQ ID NO: 97;
vii) a binding protein having
   Heavy chain as defined by SEQ ID NO: 79; and
   Light chain as defined by SEQ ID NO: 98;
viii) a binding protein having
   Heavy chain as defined by SEQ ID NO: 80; and
   Light chain as defined by SEQ ID NO: 99;
ix) a binding protein having
   Heavy chain as defined by SEQ ID NO: 81; and
   Light chain as defined by SEQ ID NO: 100;
x) a binding protein having
   Heavy chain as defined by SEQ ID NO: 82; and
   Light chain as defined by SEQ ID NO: 101;
xi) a binding protein having
   Heavy chain as defined by SEQ ID NO: 83; and
   Light chain as defined by SEQ ID NO: 102;
xii) a binding protein having
   Heavy chain as defined by SEQ ID NO: 84; and
   Light chain as defined by SEQ ID NO: 103;
xiii) a binding protein having
   Heavy chain as defined by SEQ ID NO: 85; and
   Light chain as defined by SEQ ID NO: 104;
xiv) a binding protein having
   Heavy chain as defined by SEQ ID NO: 86; and
   Light chain as defined by SEQ ID NO: 106;
xv) a binding protein having
   Heavy chain as defined by SEQ ID NO: 87; and
   Light chain as defined by SEQ ID NO: 107;
xvi) a binding protein having
   Heavy chain as defined by SEQ ID NO: 88; and
   Light chain as defined by SEQ ID NO: 108;
xviii) a binding protein having
   Heavy chain as defined by SEQ ID NO: 89; and
   Light chain as defined by SEQ ID NO: 109;
xviii) a binding protein having
   Heavy chain as defined by SEQ ID NO: 90; and
   Light chain as defined by SEQ ID NO: 110;
xix) a binding protein having
   Heavy chain as defined by SEQ ID NO: 91; and
   Light chain as defined by SEQ ID NO: 111;
xx) a binding protein having
   Heavy chain as defined by SEQ ID NO: 92; and
   Light chain as defined by SEQ ID NO: 112;
xxi) a binding protein having
   Heavy chain as defined by SEQ ID NO: 149; and
   Light chain as defined by SEQ ID NO: 150;
and sequences having 80% or more, such as 85%, 90%, 95% or more, identity thereto.

21. The binding protein of items 20-21, wherein the CDR sequences comprise no variations in the amino acid sequence, or wherein the sequence variation of the CDR amino acid sequences is at most 5%, such as 4%, 3%, 2%, 1% or less.

22. A conjugated binding protein comprising:
(i) at least one binding protein as defined in any one of items 1 to 21; and
(ii) at least one agent.

23. The conjugated binding protein of item 22, wherein the at least one agent is joined to the binding protein, wherein the binding protein and agent are directly or indirectly joined.

24. The conjugated binding protein of items 22 or 23, wherein the agent is radioisotope, a photoactivatable compound, a radioactive compound, an enzyme, a fluorescent dye, a biotin molecule, a toxin, a cytotoxic agent, a prodrug, a binding molecule with a different specificity, a cytokine or another immunomodulatory polypeptide.

25. The conjugated binding protein of items 22-24, wherein the agent is a therapeutic agent.

26. The conjugated binding protein of item 25, wherein the therapeutic agent is a cytotoxic agent that comprises or consists of one or more radioisotopes.

27. The conjugated binding protein of items 24 or 26, wherein the one or more radioisotopes is or are each independently selected from the group consisting of beta-emitters, auger-emitters, conversion electron-emitters, alpha-emitters, and low photon energy-emitters.

28. The conjugated binding protein of item 27, wherein the one or more radioisotopes each independently have an emission pattern of locally absorbed energy that creates a high dose absorbance in the vicinity of the agent.

29. The conjugated binding protein of items 27 or 28, wherein the one or more radioisotopes are each independently selected from the group consisting of long-range beta-emitters, such as ⁹⁰Y, ³²P, ¹⁸⁶Re/¹⁸⁸Re; ¹⁶Ho, ⁷⁶As/⁷⁷As, ¹⁵³Sm; medium range beta-emitters, such as ¹³¹I, ¹⁷⁷Lu, ⁶⁷Cu, ¹⁶¹Tb, ⁴⁷Sc; low-energy beta-emitters, such as ⁴⁵Ca, ³⁵S or ¹⁴C; conversion or auger-emitters, such as ⁵¹Cr, ⁶⁷Ga, ⁹⁹TC^{m}, ¹¹¹In, ¹²³I, ¹²⁵I, ²⁰¹Tl, and alpha-emitters, such as ²¹²Bi, ²¹²Pb, ²¹³Bi, ²²³Ac, ²²⁵Ac, ²²⁷Th, ¹⁴⁹Tb and ²¹¹At.

30. The conjugated binding protein of items 24 or 26-29, wherein the radioisotope is ¹⁷⁷Lu.

31. The conjugated binding protein of item 25, wherein the therapeutic agent is a cytotoxic agent that comprises or consists of one or more cytotoxic drugs.

32. The conjugated binding protein of item 31, wherein the one or more cytotoxic agent is selected from a cytostatic drug, a toxin, and a chemotherapeutic agent.

33. The conjugated binding protein of items 22-24, wherein the agent is a detectable agent.

34. The conjugated binding protein of item 33, wherein the detectable agent is selected from the group consisting of radioisotopes, enzymes, fluorescent molecules, dyes, digoxigenin, and biotin, among others.

35. The conjugated binding protein of items 33 or 34, wherein the detectable agent comprises or consists of a radioisotope.

36. The conjugated binding protein of items 34 or 35, wherein the radioisotope is selected from the group consisting of ¹¹¹In, ^{99m}Tc, ⁶⁷Ga, ⁶⁸Ga, ⁷²As, ⁸⁹Zr, ¹²³I, ¹²⁵I, ¹²⁴I, ⁴⁷Sc and ²⁰¹Tl.

37. The conjugated binding protein of items 24, 26-28 or 34-35, wherein the conjugated binding protein comprises a pair of detectable and cytotoxic radioisotopes, such as ⁸⁶γ/⁹⁰γ, ¹¹¹In/¹⁷⁷Lu or ¹²⁵I/²¹¹At.

38. The conjugated binding protein of item 37, wherein the radioisotope is capable of simultaneously acting in a multi-modal manner as a detectable agent and also as a cytotoxic agent.

39. The conjugated binding protein of items 33-38, wherein the detectable agent is detectable by an imaging technique such as SPECT, PET, MRI, optical or ultrasound imaging.

40. The conjugated binding protein of items 22 or 23, wherein the agent is indirectly joined to the binding protein via a linker.

41. The conjugated binding protein of item 40, wherein the linker is in the form of a chelator.

42. The conjugated binding protein of item 41, wherein the chelator is selected from the group consisting of derivatives of 1,4,7,10-tetraazacyclododecane-1,4,7,10, tetraacetic acid (DOTA), derivatives of deferoxamine (DFO), derivatives of diethylenetriaminepentaacetic avid (DTPA), derivatives of S-2-(4-Isothiocyanatobenzyl)-1,4,7-triazacyclononane-1,4,7-triacetic acid (NOTA), derivatives of (tBu) 4 (1-(1-carboxy-3-carbotertbutoxypropyl)-4,7,10-(carbotertbut-oxymethyl)-1,4,7,10-tetraazacyclododecane) (DOTAGA), derivatives of 1,4,8,11-tetraazacyclodocedan-1,4,8,11-tetraacetic acid (TETA), derivatives of 1,4,7-triazacyclononane, 1-glutaric acid-4,7-acetic acid (NODAGA), derivatives of 1,4,7-Triazacyclononane-1,4,7-triacetic acid (NOTA).

43. The conjugated binding protein of item 40, wherein the linker is a molecular attachment tag for a pretargeting treatment using a secondary binding molecule, wherein the secondary binding molecule is joined to the agent and wherein the secondary binding molecule binds the attachment tag of the binding protein, thereby linking the agent to the binding protein to form a conjugated binding protein.

44. A cell engineered to express a chimeric antigen receptor (CAR), wherein the CAR comprises an antigen-binding domain, a transmembrane domain connected to the antigen binding domain by a hinge region, and an intracellular domain optionally connected to one or more co-stimulatory domains, wherein the antigen binding domain comprises the binding protein of any one of items 1 to 15.

45. The cell of item 44, wherein the cell is a human cell.

46. The cell of items 44 or 45, wherein the cell is an immune effector cell, such as a T cell, an NK cell or a macrophage.

47. A pharmaceutical composition containing a binding protein of any one of items 1-21, a conjugated binding protein of any one of items 22-32 or 37-43, or a cell of any one of items 44-46, and a pharmaceutically acceptable carrier or excipient.

48. The binding protein of any one of items 1-21, a conjugated binding protein of any one of items 22-32 or 37-43, a cell of any one of items 44-46, or a pharmaceutical composition of item 47, for use in therapy.

49. The binding protein, conjugated binding protein, cell, or pharmaceutical composition according to item 48, for use in cancer therapy.

50. A method of treating a subject in need thereof, comprising administering a therapeutically effective amount of a binding protein of any one of items 1-21, a conjugated binding protein of any one of items 22-32 or 37-43, a cell of any one of items 44-46, or a pharmaceutical composition of item 47.

51. A method of treating a subject in need thereof, the method comprising:
administering therapeutically effective amount of a first binding protein of any one of items 1-21, the binding protein further comprising a molecular attachment tag;
allowing any unbound binding proteins to leave the circulation of the subject;
administering a therapeutically effective amount of a second molecule, wherein the second molecule is joined to a therapeutic agent, and wherein the second molecule bind the first binding protein, thereby delivering the therapeutic agent to the CD44v6 epitope bound by first binding protein.

52. The method according to items 50 or 51, wherein the subject has a disorder characterized by expression of the CD44 variant CD44v6

53. The method according to item 52, wherein the disorder is cancer or another angiogenesis related disorder.

54. Use of a binding protein of any one of items 1-21, a conjugated binding protein of any one of items 22-32 or 37-43, a cell of any one of items 44-46, or a pharmaceutical composition of item 47, for the treatment, prevention or diagnosis of cancer.

55. The binding protein, conjugated binding protein, cell, or pharmaceutical composition according to item 49, the method of item 53, or the use of item 54, wherein the cancer is selected from the group consisting of: advanced thyroid cancer, head and neck cancer, pancreatic cancer, squamous cell carcinoma, Hodgkin lymphoma, colorectal cancer, liver cancer, cervical cancer, gastric cancer, ovarian cancer, lung cancer, bladder cancer, acute myeloid leukemia, chronic lymphocytic leukemia, multiple myeloma, breast cancer, hepatocellular cancer, and esophageal cancer, and metastatic cancers of the brain.

56. An in vitro method for detecting expression of the CD44 variant CD44v6, the method comprising:
(i) contacting a binding protein according to items 1-21, or a conjugated binding protein of items 22-24 or 33-43, to a biological sample, such as a tissue sample or liquid, obtained from a subject, such that the binding protein or conjugated binding protein binds to an epitope of CD44v6 as defined by SEQ ID NO: 7 if present in the biological sample;
(ii) washing the biological sample to remove unbound binding proteins or conjugated binding proteins; and
(iii) detecting any binding proteins or conjugated binding proteins that have bound the epitope in the biological sample.

57. An in vivo method for detecting expression of the CD44 variant CD44v6, the method comprising:
(i) administering a conjugated binding protein of items 22-24 or 33-43, to a subject, wherein the conjugated binding protein binds an epitope of CD44v6 as defined by SEQ ID NO: 7; and
(ii) detecting that the conjugated binding protein has bound a cell expressing the epitope. 58. The in vivo method of item 57, wherein the conjugated binding protein comprises one or more 111 In radioisotope atoms.

59. A conjugated binding protein comprising:
(iii) at least one binding protein as defined in any one of items 1 to 14; and
(iv) at least one ¹⁷⁷Lu radioisotope atom joined to the binding protein; for use in the treatment of advanced thyroid cancer.

60. The binding protein of items 1-21, where in the dissociation constant Kp as measured on cells from the BHT-101 cell line is below 9 nM, such as below 8, 7, 6, 5, 4, 3, 2, or 1 nM, preferably below 1 nM, such as below 0.8, 0.7, 0.6, 0.5, 0.4, 0.3 or 0.2 nM.

61. The binding protein of item 60, where in the dissociation constant Kp as measured on cells from the BHT-101 cell line is below 0.2 nM, such that K_{D}<0.2 nM.

1. A binding protein that specifically binds CD44v6 and comprises a binding domain of an antibody, the binding domain comprising a heavy chain variable domain (VH) and a light chain variable domain (VL) or derivatives thereof, each comprising three complementarity determining regions (CDRs), wherein the amino acid sequences of the CDRs are selected from the group comprising:
VHCDR1 as defined by SEQ ID NO: 1;
VHCDR2 as defined by SEQ ID NO: 2;
VHCDR3 as defined by SEQ ID NO: 3;
VLCDR1 as defined by SEQ ID NO: 4;
VLCDR2 as defined by X₁AS, where X₁ may be T, A, or S;
VLCDR3 as defined by SEQ ID NO: 6;
and CDR sequences having 95% or more, such as 96%, 97%, 98%, 99% or more, identity thereto, wherein said binding protein recognises an epitope of CD44v6 as defined by SEQ ID NO: 7.

2. The binding protein of claim 1, wherein VHCDR1, VHCDR2 and VLCDR2 are present next to specific framework amino acids, wherein the CDR and framework amino acid (faa) sequences are selected from the group comprising:
VHCDR1 and faa defined by SEQ ID NO: 8;
VHCDR2 and faa as defined by SEQ ID NO: 9;
VLCDR2 and faa as defined by SEQ ID NO: 10;
and CDR sequences having 95% or more, such as 96%, 97%, 98%, 99% or more, identity thereto.

3. The binding protein of claim 1, wherein the CDRs are individually selected from the group comprising:
VHCDR1 is selected from SEQ ID NO: 11-18;
VHCDR2 is selected from SEQ ID NO: 19-25, 32;
VHCDR3 as defined by SEQ ID NO: 3;
VLCDR1 is selected from SEQ ID NO: 26-31;
VLCDR2 is selected from TAS, SAS and AAS;
VLCDR3 as defined by SEQ ID NO:6;
and CDR sequences having 95% or more, such as 96%, 97%, 98%, 99% or more, identity thereto.

4. The binding protein of claims 1-3, wherein the VH sequence comprises an amino acid sequence selected from the group consisting of SEQ ID NO: 35-54 and 147, and sequences having 80% or more, such as 85%, 90%, 95% or more, identity thereto, and wherein the VL sequence comprises an amino acid sequence selected from the group consisting of SEQ ID NO: 55-74 and 148, and sequences having 80% or more, such as 85%, 90%, 95% or more, identity thereto.

5. The binding protein of claim 1, wherein the binding protein is a monoclonal antibody, or an antigen-binding fragment selected from the group consisting of Fv fragments, such as scFv fragments, Fab-like fragments, such as Fab or F(ab′)₂ fragments, and domain antibodies.

6. The binding protein of claims 1-5, wherein the binding molecule is a monoclonal antibody of the IgG1 isotype, such as an IgG1 LALA antibody or IgG1 LALA IAHA antibody.

7. The binding protein of claims 1-6, wherein the binding protein is human or of human origin.

8. The binding protein of claims 5-7, wherein the antibody comprises a heavy chain and a light chain, the heavy chain comprising an amino acid sequence selected from the group consisting of SEQ ID NO: 34, 75-93 and 149 and sequences having 80% or more, such as 85%, 90%, 95% or more, identity thereto; and the light chain comprising an amino acid sequence selected from the group consisting of SEQ ID NO: 94-113 and 150, and sequences having 80% or more, such as 85%, 90%, 95% or more, identity thereto.

9. The binding protein of claim 4 or 8, wherein the CDR sequences comprise no variations in the amino acid sequence, or wherein the sequence variation of the CDR amino acid sequences is at most 5%, such as 4%, 3%, 2%, 1% or less.

10. The binding protein of claims 1-9, where the binding proteins bind to BHT-101 cells such that the KD value of the interaction is at most 1 nM, such as preferably at most 0.2 nM.

11. A conjugated binding protein comprising:
(v) at least one binding protein as defined in any one of claims 1 to 10; and
(vi) at least one agent.

12. The conjugated binding protein of claim 11, wherein the at least one agent is a therapeutic agent or a detectable agent.

13. The conjugated binding protein of claim 12, wherein the at least one therapeutic agent is one or more cytotoxic agent, such as a radioisotope, a cytostatic drug, a toxin, or a chemotherapeutic agent, and wherein the at least one detectable agent is one or more radioisotope, enzyme, fluorescent molecule, dye, digoxigenin, or biotin.

14. The conjugated binding protein of claim 13, wherein the radioisotopes used as therapeutic agents are selected from the group consisting of medium range beta-emitters, such as ¹⁷⁷Lu, ¹³¹I, ⁶⁷Cu, ¹⁶¹Tb, ⁴⁷Sc; long-range beta-emitters, such as ⁹⁰Y, ³²P, ¹⁸⁶Re/¹⁸⁸Re; ¹⁶⁶Ho, ⁷⁶As/⁷⁷As, ¹⁵³Sm; low-energy beta-emitters, such as ⁴⁵Ca, ³⁵S or ¹⁴C; conversion or auger-emitters, such as ⁵¹Cr, ⁶⁷Ga, ⁹⁹TC^{m}, ¹¹¹In, ¹²³I, ¹²⁵I, ²⁰¹Tl, and alpha-emitters, such as ²¹²Bi, ²¹²Pb, ²¹³Bi, ²²³Ac, ²²⁵Ac, ²²⁷Th, ¹⁴⁹Tb and ²¹¹At, and wherein the radioisotopes used as detectable agents are selected from the group consisting of ¹¹¹In, ^{99m}Tc, ⁶⁷Ga, ⁶⁸Ga, ⁷²As, ⁸⁹Zr, ¹²³I, ¹²⁵I, ¹²⁴I, ⁴⁷Sc and ²⁰¹Tl, or wherein the conjugated binding protein comprises a pair of detectable and cytotoxic radioisotopes, the radioisotope pairs are selected from ¹¹¹In/¹⁷⁷Lu, ⁸⁶γ/⁹⁰γ and ¹²⁵I/²¹¹At.

15. The conjugated binding protein of claims 11-14, wherein the agent is indirectly joined to the binding protein via a linker, such as a chelator, wherein the chelator is selected from the group consisting of derivatives of 1,4,7,10-tetraazacyclododecane-1,4,7,10, tetraacetic acid (DOTA), derivatives of deferoxamine (DFO), derivatives of diethylenetriaminepentaacetic avid (DTPA), derivatives of S-2-(4-Isothiocyanatobenzyl)-1,4,7-triazacyclononane-1,4,7-triacetic acid (NOTA), derivatives of (tBu) 4 (1-(1-carboxy-3-carbotertbutoxypropyl)-4,7,10-(carbotertbut-oxymethyl)-1,4,7,10-tetraazacyclododecane) (DOTAGA), derivatives of 1,4,8,11-tetraazacyclodocedan-1,4,8,11-tetraacetic acid (TETA), derivatives of 1,4,7-triazacyclononane, 1-glutaric acid-4,7-acetic acid (NODAGA), derivatives of 1,4,7-Triazacyclononane-1,4,7-triacetic acid (NOTA).

16. A cell engineered to express a chimeric antigen receptor (CAR), wherein the CAR comprises an antigen-binding domain, a transmembrane domain connected to the antigen binding domain by a hinge region, and an intracellular domain optionally connected to one or more co-stimulatory domains, wherein the antigen binding domain comprises the binding protein of any one of claims 1-5.

17. A pharmaceutical composition containing a binding protein of any one of claims 1-10, a conjugated binding protein of any one of claims 11-15, or a cell of claim 16, and a pharmaceutically acceptable carrier or excipient.

18. The binding protein of any one of claims 1-10, the conjugated binding protein of any one of claims 11-15, the cell of claim 16, or the pharmaceutical composition of claim 17, for use in therapy.

19. The binding protein, conjugated binding protein, cell, or pharmaceutical composition according to claim 18, for use in cancer therapy.

20. The binding protein, conjugated binding protein, cell, or pharmaceutical composition for use according to claim 19, wherein the cancer is advanced thyroid cancer.

21. An in vitro method for detecting expression of the CD44 variant CD44v6, the method comprising:
(i) contacting a binding protein according to claims 1-10, or a conjugated binding protein of claims 11-15, to a biological sample, such as a tissue sample or liquid, obtained from a subject, such that the binding protein or conjugated binding protein binds to an epitope of CD44v6 as defined by SEQ ID NO: 7 if present in the biological sample;
(ii) washing the biological sample to remove unbound binding proteins or conjugated binding proteins; and
(iii) detecting any binding proteins or conjugated binding proteins that have bound the epitope in the biological sample.

22. An in vivo method for detecting expression of the CD44 variant CD44v6, the method comprising:
detecting that a conjugated binding protein of claims 11-15 has bound a cell expressing an epitope of CD44v6 as defined by SEQ ID NO: 7.


SEQUENCE LISTING (150 sequences)

SEQ ID NO: 1 - protein, 8 aa, synthetic construct
  VARIANT 3: note = T or S
  VARIANT 5: note = S, R or G
  VARIANT 6: note = S or N
  VARIANT 7: note = Y or F
GFXFXXXA

SEQ ID NO: 2 - protein, 8 aa, synthetic construct
  VARIANT 3: note = G or A
  VARIANT 4: note = S or G
  VARIANT 6: note = G, T, S, Y or R
ISXXGXST

SEQ ID NO: 3 - protein, 18 aa, synthetic construct
ARHYYSDSDYRSSAAMDY

SEQ ID NO: 4 - skipped

SEQ ID NO: 5 - protein, 10 aa, Homo sapiens
WFGNRWHEGY

SEQ ID NO: 6 - protein, 8 aa, synthetic construct
QQDLYLLT

SEQ ID NO: 7 - protein, 6 aa, Homo sapiens
WFGNRW

SEQ ID NO: 8 - protein, 10 aa, synthetic construct
  VARIANT 3: note = T or S
  VARIANT 5: note = S, G or R
  VARIANT 6: note = S or N
  VARIANT 7: note = Y or F
GFXFXXXAMS

SEQ ID NO: 9 - protein, 10 aa, synthetic construct
  VARIANT 1: note = A or T
  VARIANT 4: note = A or G
  VARIANT 5: note = G or S
  VARIANT 7: note = G, T, Y, R or S
  VARIANT 10: note = Y or F
XISXXGXSTX

SEQ ID NO: 10 - skipped

SEQ ID NO: 11 - protein, 8 aa, synthetic construct
GFSFGSYA

SEQ ID NO: 12 - protein, 8 aa, synthetic construct
GFTFSSYA

SEQ ID NO: 13 - protein, 8 aa, synthetic construct
GFSFSSYA

SEQ ID NO: 14 - protein, 8 aa, synthetic construct
GFSFSSFA

SEQ ID NO: 15 - protein, 8 aa, synthetic construct
GFSFGSFA

SEQ ID NO: 16 - protein, 8 aa, synthetic construct
GFSFRSFA

SEQ ID NO: 17 - protein, 8 aa, synthetic construct
GFTFRSFA

SEQ ID NO: 18 - protein, 8 aa, synthetic construct
GFSFRNFA

SEQ ID NO: 19 - protein, 8 aa, synthetic construct
ISAGGSST

SEQ ID NO: 20 - protein, 8 aa, synthetic construct
ISGGGSST

SEQ ID NO: 21 - protein, 8 aa, synthetic construct
ISAGGTST

SEQ ID NO: 22 - protein, 8 aa, synthetic construct
ISAGGYST

SEQ ID NO: 23 - protein, 8 aa, synthetic construct
ISGGGYST

SEQ ID NO: 24 - protein, 8 aa, synthetic construct
ISGGGGST

SEQ ID NO: 25 - protein, 8 aa, synthetic construct
ISGSGRST

SEQ ID NO: 26 - protein, 6 aa, synthetic construct
QSISSY

SEQ ID NO: 27 - protein, 6 aa, synthetic construct
QSIGSY

SEQ ID NO: 28 - protein, 6 aa, synthetic construct
QSIANY

SEQ ID NO: 29 - protein, 6 aa, synthetic construct
QTIGSY

SEQ ID NO: 30 - protein, 6 aa, synthetic construct
QTIASY

SEQ ID NO: 31 - protein, 6 aa, synthetic construct
QNISSY

SEQ ID NO: 32 - protein, 8 aa, synthetic construct
ISGSGGST

SEQ ID NO: 33 - protein, 10 aa, synthetic construct
AISGSGGSTY

SEQ ID NO: 34 - protein, 455 aa, synthetic construct
EVQLLESGGGLVQPGGSLRLSCAASGFSFRSFAMSWVRQAPGKGLEWVSAISGGGSSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 125 aa, synthetic construct
EVQLLESGGGLVQPGGSLRLSCAASGFSFGSYAMSWVRQAPGKGLEWVSAISAGGSSTFYADSVKGRFTISRDNSKNTLYLQMNSLRAEDTAVYYCARHYYSDSDYRSSAAMDYWGQGTLVTVSS

SEQ ID NO: 36 - protein, 125 aa, synthetic construct
EVQLLESGGGLVQPGGSLRLSCAASGFSFGSYAMSWVRQAPGKGLEWVSAISGGGSSTFYADSVKGRFTISRDNSKNTLYLQMNSLRAEDTAVYYCARHYYSDSDYRSSAAMDYWGQGTLVTVSS

SEQ ID NO: 37 - protein, 125 aa, synthetic construct
EVQLLESGGGLVQPGGSLRLSCAASGFTFSSYAMSWVRQAPGKGLEWVSAISGGGSSTYYADSVKGRFTISRDNSKNTLYLQMNSLRAEDTAVYYCARHYYSDSDYRSSAAMDYWGQGTLVTVSS

SEQ ID NO: 38 - protein, 125 aa, synthetic construct
EVQLLESGGGLVQPGGSLRLSCAASGFTFSSYAMSWVRQAPGKGLEWVSAISAGGTSTYYADSVKGRFTISRDNSKNTLYLQMNSLRAEDTAVYYCARHYYSDSDYRSSAAMDYWGQGTLVTVSS

SEQ ID NO: 39 - protein, 125 aa, synthetic construct
EVQLLESGGGLVQPGGSLRLSCAASGFTFSSYAMSWVRQAPGKGLEWVSAISAGGYSTYYADSVKGRFTISRDNSKNTLYLQMNSLRAEDTAVYYCARHYYSDSDYRSSAAMDYWGQGTLVTVSS

SEQ ID NO: 40 - protein, 125 aa, synthetic construct
EVQLLESGGGLVQPGGSLRLSCAASGFSFSSYAMSWVRQAPGKGLEWVSAISAGGSSTFYADSVKGRFTISRDNSKNTLYLQMNSLRAEDTAVYYCARHYYSDSDYRSSAAMDYWGQGTLVTVSS

SEQ ID NO: 41 - protein, 125 aa, synthetic construct
EVQLLESGGGLVQPGGSLRLSCAASGFSFGSYAMSWVRQAPGKGLEWVSAISAGGSSTYYADSVKGRFTISRDNSKNTLYLQMNSLRAEDTAVYYCARHYYSDSDYRSSAAMDYWGQGTLVTVSS

SEQ ID NO: 42 - protein, 125 aa, synthetic construct
EVQLLESGGGLVQPGGSLRLSCAASGFSFSSFAMSWVRQAPGKGLEWVSAISAGGSSTFYADSVKGRFTISRDNSKNTLYLQMNSLRAEDTAVYYCARHYYSDSDYRSSAAMDYWGQGTLVTVSS

SEQ ID NO: 43 - protein, 125 aa, synthetic construct
EVQLLESGGGLVQPGGSLRLSCAASGFSFSSFAMSWVRQAPGKGLEWVSAISAGGSSTYYADSVKGRFTISRDNSKNTLYLQMNSLRAEDTAVYYCARHYYSDSDYRSSAAMDYWGQGTLVTVSS

SEQ ID NO: 44 - protein, 125 aa, synthetic construct
EVQLLESGGGLVQPGGSLRLSCAASGFTFSSYAMSWVRQAPGKGLEWVSAISAGGSSTFYADSVKGRFTISRDNSKNTLYLQMNSLRAEDTAVYYCARHYYSDSDYRSSAAMDYWGQGTLVTVSS

SEQ ID NO: 45 - protein, 125 aa, synthetic construct
EVQLLESGGGLVQPGGSLRLSCAASGFSFGSFAMSWVRQAPGKGLEWVSAISAGGSSTFYADSVKGRFTISRDNSKNTLYLQMNSLRAEDTAVYYCARHYYSDSDYRSSAAMDYWGQGTLVTVSS

SEQ ID NO: 46 - protein, 125 aa, synthetic construct
EVQLLESGGGLVQPGGSLRLSCAASGFSFRSFAMSWVRQAPGKGLEWVSAISGGGSSTFYADSVKGRFTISRDNSKNTLYLQMNSLRAEDTAVYYCARHYYSDSDYRSSAAMDYWGQGTLVTVSS

SEQ ID NO: 47 - protein, 125 aa, synthetic construct
EVQLLESGGGLVQPGGSLRLSCAASGFTFRSFAMSWVRQAPGKGLEWVSTISAGGSSTFYADSVKGRFTISRDNSKNTLYLQMNSLRAEDTAVYYCARHYYSDSDYRSSAAMDYWGQGTLVTVSS

SEQ ID NO: 48 - protein, 125 aa, synthetic construct
EVQLLESGGGLVQPGGSLRLSCAASGFSFGSFAMSWVRQAPGKGLEWVSAISAGGSSTYYADSVKGRFTISRDNSKNTLYLQMNSLRAEDTAVYYCARHYYSDSDYRSSAAMDYWGQGTLVTVSS

SEQ ID NO: 49 - protein, 125 aa, synthetic construct
EVQLLESGGGLVQPGGSLRLSCAASGFTFSSYAMSWVRQAPGKGLEWVSAISAGGSSTYYADSVKGRFTISRDNSKNTLYLQMNSLRAEDTAVYYCARHYYSDSDYRSSAAMDYWGQGTLVTVSS

SEQ ID NO: 50 - protein, 125 aa, synthetic construct
EVQLLESGGGLVQPGGSLRLSCAASGFTFSSYAMSWVRQAPGKGLEWVSAISGGGYSTYYADSVKGRFTISRDNSKNTLYLQMNSLRAEDTAVYYCARHYYSDSDYRSSAAMDYWGQGTLVTVSS

SEQ ID NO: 51 - protein, 125 aa, synthetic construct
EVQLLESGGGLVQPGGSLRLSCAASGFSFGSFAMSWVRQAPGKGLEWVSAISGGGSSTYYADSVKGRFTISRDNSKNTLYLQMNSLRAEDTAVYYCARHYYSDSDYRSSAAMDYWGQGTLVTVSS

SEQ ID NO: 52 - protein, 125 aa, synthetic construct
EVQLLESGGGLVQPGGSLRLSCAASGFSFRNFAMSWVRQAPGKGLEWVSAISGGGGSTFYADSVKGRFTISRDNSKNTLYLQMNSLRAEDTAVYYCARHYYSDSDYRSSAAMDYWGQGTLVTVSS

SEQ ID NO: 53 - protein, 125 aa, synthetic construct
EVQLLESGGGLVQPGGSLRLSCAASGFTFSSYAMSWVRQAPGKGLEWVSAISGSGRSTFYADSVKGRFTISRDNSKNTLYLQMNSLRAEDTAVYYCARHYYSDSDYRSSAAMDYWGQGTLVTVSS

SEQ ID NO: 54 - protein, 125 aa, synthetic construct
EVQLLESGGGLVQPGGSLRLSCAASGFTFRSFAMSWVRQAPGKGLEWVSAISGGGSSTFYADSVKGRFTISRDNSKNTLYLQMNSLRAEDTAVYYCARHYYSDSDYRSSAAMDYWGQGTLVTVSS

SEQ ID NO: 55 - protein, 106 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQDLYLLTFGQGTKLEIK

SEQ ID NO: 56 - protein, 106 aa, synthetic construct
DIQMTQSPSSLSASVGDRVTITCRASQSISSYLNWYQQKPGKAPKLLIYSASSLQSGVPSRFSGSGSGTDFTLTISSLQPEDFATYYCQQDLYLLTFGQGTKLEIK

SEQ ID NO: 57 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQDLYLLTFGQGTKLEIK

SEQ ID NO: 58 - protein, 106 aa, synthetic construct; identical to SEQ ID NO: 57

SEQ ID NO: 59 - protein, 106 aa, synthetic construct
DIQMTQSPSSLSASVGDRVTITCRASQSIGSYLNWYQQKPGKAPKLLIYTASTLQSGVPSRFSGSGSGTDFTLTISSLQPEDFATYYCQQDLYLLTFGQGTKLEIK

SEQ ID NO: 60 - protein, 106 aa, synthetic construct
DIQMTQSPSSLSASVGDRVTITCRASQSIGSYLNWYQQKPGKAPKLLIYSASSLQSGVPSRFSGSGSGTDFTLTISSLQPEDFATYYCQQDLYLLTFGQGTKLEIK

SEQ ID NO: 61 - protein, 106 aa, synthetic construct; identical to SEQ ID NO: 60

SEQ ID NO: 62 - protein, 106 aa, synthetic construct
DIQMTQSPSSLSASVGDRVTITCRASQSIGSYLNWYQQKPGKAPKLLIYSASNLQSGVPSRFSGSGSGTDFTLTISSLQPEDFATYYCQQDLYLLTFGQGTKLEIK

SEQ ID NO: 63 - protein, 106 aa, synthetic construct; identical to SEQ ID NO: 57

SEQ ID NO: 64 - protein, 106 aa, synthetic construct; identical to SEQ ID NO: 57

SEQ ID NO: 65 - protein, 106 aa, synthetic construct; identical to SEQ ID NO: 57

SEQ ID NO: 66 - protein, 106 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQDLYLLTFGQGTKLEIK

SEQ ID NO: 67 - protein, 106 aa, synthetic construct
DIQMTQSPSSLSASVGDRVTITCRASQSIANYLNWYQQKPGKAPKLLIYAASTLQSGVPSRFSGSGSGTDFTLTISSLQPEDFATYYCQQDLYLLTFGQGTKLEIK

SEQ ID NO: 68 - protein, 106 aa, synthetic construct; identical to SEQ ID NO: 57

SEQ ID NO: 69 - protein, 106 aa, synthetic construct; identical to SEQ ID NO: 57

SEQ ID NO: 70 - protein, 106 aa, synthetic construct
DIQMTQSPSSLSASVGDRVTITCRASQTIGSYLNWYQQKPGKAPKLLIYSASILQSGVPSRFSGSGSGTDFTLTISSLQPEDFATYYCQQDLYLLTFGQGTKLEIK

SEQ ID NO: 71 - protein, 106 aa, synthetic construct
DIQMTQSPSSLSASVGDRVTITCRASQTIASYLNWYQQKPGKAPKLLIYTASNLQSGVPSRFSGSGSGTDFTLTISSLQPEDFATYYCQQDLYLLTFGQGTKLEIK

SEQ ID NO: 72 - protein, 106 aa, synthetic construct
DIQMTQSPSSLSASVGDRVTITCRASQNISSYLNWYQQKPGKAPKLLIYSASILQSGVPSRFSGSGSGTDFTLTISSLQPEDFATYYCQQDLYLLTFGQGTKLEIK

SEQ ID NO: 73 - protein, 106 aa, synthetic construct; identical to SEQ ID NO: 57

SEQ ID NO: 74 - protein, 106 aa, synthetic construct; identical to SEQ ID NO: 60

SEQ ID NO: 75 - protein, 455 aa, synthetic construct
EVQLLESGGGLVQPGGSLRLSCAASGFSFGSYAMSWVRQAPGKGLEWVSAISAGGSSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 455 aa, synthetic construct
EVQLLESGGGLVQPGGSLRLSCAASGFSFGSYAMSWVRQAPGKGLEWVSAISGGGSSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 455 aa, synthetic construct
EVQLLESGGGLVQPGGSLRLSCAASGFTFSSYAMSWVRQAPGKGLEWVSAISGGGSSTYYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 455 aa, synthetic construct
EVQLLESGGGLVQPGGSLRLSCAASGFTFSSYAMSWVRQAPGKGLEWVSAISAGGTSTYYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 455 aa, synthetic construct
EVQLLESGGGLVQPGGSLRLSCAASGFTFSSYAMSWVRQAPGKGLEWVSAISAGGYSTYYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 455 aa, synthetic construct
EVQLLESGGGLVQPGGSLRLSCAASGFSFSSYAMSWVRQAPGKGLEWVSAISAGGSSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 455 aa, synthetic construct
EVQLLESGGGLVQPGGSLRLSCAASGFSFGSYAMSWVRQAPGKGLEWVSAISAGGSSTYYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 455 aa, synthetic construct
EVQLLESGGGLVQPGGSLRLSCAASGFSFSSFAMSWVRQAPGKGLEWVSAISAGGSSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 455 aa, synthetic construct
EVQLLESGGGLVQPGGSLRLSCAASGFSFSSFAMSWVRQAPGKGLEWVSAISAGGSSTYYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 455 aa, synthetic construct
EVQLLESGGGLVQPGGSLRLSCAASGFTFSSYAMSWVRQAPGKGLEWVSAISAGGSSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 455 aa, synthetic construct
EVQLLESGGGLVQPGGSLRLSCAASGFSFGSFAMSWVRQAPGKGLEWVSAISAGGSSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 455 aa, synthetic construct
EVQLLESGGGLVQPGGSLRLSCAASGFTFRSFAMSWVRQAPGKGLEWVSTISAGGSSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 455 aa, synthetic construct
EVQLLESGGGLVQPGGSLRLSCAASGFSFGSFAMSWVRQAPGKGLEWVSAISAGGSSTYYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 455 aa, synthetic construct
EVQLLESGGGLVQPGGSLRLSCAASGFTFSSYAMSWVRQAPGKGLEWVSAISAGGSSTYYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 455 aa, synthetic construct
EVQLLESGGGLVQPGGSLRLSCAASGFTFSSYAMSWVRQAPGKGLEWVSAISGGGYSTYYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 455 aa, synthetic construct
EVQLLESGGGLVQPGGSLRLSCAASGFSFGSFAMSWVRQAPGKGLEWVSAISGGGSSTYYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 455 aa, synthetic construct
EVQLLESGGGLVQPGGSLRLSCAASGFSFRNFAMSWVRQAPGKGLEWVSAISGGGGSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 455 aa, synthetic construct
EVQLLESGGGLVQPGGSLRLSCAASGFTFSSYAMSWVRQAPGKGLEWVSAISGSGRSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 455 aa, synthetic construct
EVQLLESGGGLVQPGGSLRLSCAASGFTFRSFAMSWVRQAPGKGLEWVSAISGGGSSTFYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 213 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 213 aa, synthetic construct
DIQMTQSPSSLSASVGDRVTITCRASQSISSYLNWYQQKPGKAPKLLIYSASSLQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 213 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 213 aa, synthetic construct; identical to SEQ ID NO: 96

SEQ ID NO: 98 - protein, 213 aa, synthetic construct
DIQMTQSPSSLSASVGDRVTITCRASQSIGSYLNWYQQKPGKAPKLLIYTASTLQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 213 aa, synthetic construct
DIQMTQSPSSLSASVGDRVTITCRASQSIGSYLNWYQQKPGKAPKLLIYSASSLQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 213 aa, synthetic construct; identical to SEQ ID NO: 99

SEQ ID NO: 101 - protein, 213 aa, synthetic construct
DIQMTQSPSSLSASVGDRVTITCRASQSIGSYLNWYQQKPGKAPKLLIYSASNLQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 213 aa, synthetic construct; identical to SEQ ID NO: 96

SEQ ID NO: 103 - protein, 213 aa, synthetic construct; identical to SEQ ID NO: 96

SEQ ID NO: 104 - protein, 213 aa, synthetic construct; identical to SEQ ID NO: 96

SEQ ID NO: 105 - protein, 213 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 213 aa, synthetic construct
DIQMTQSPSSLSASVGDRVTITCRASQSIANYLNWYQQKPGKAPKLLIYAASTLQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 213 aa, synthetic construct; identical to SEQ ID NO: 96

SEQ ID NO: 108 - protein, 213 aa, synthetic construct; identical to SEQ ID NO: 96

SEQ ID NO: 109 - protein, 213 aa, synthetic construct
DIQMTQSPSSLSASVGDRVTITCRASQTIGSYLNWYQQKPGKAPKLLIYSASILQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 213 aa, synthetic construct
DIQMTQSPSSLSASVGDRVTITCRASQTIASYLNWYQQKPGKAPKLLIYTASNLQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 213 aa, synthetic construct
DIQMTQSPSSLSASVGDRVTITCRASQNISSYLNWYQQKPGKAPKLLIYSASILQSGVPSRFSGSGSGTDFTLTISSLQPEDFATYYCQQDLYLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 213 aa, synthetic construct; identical to SEQ ID NO: 96

SEQ ID NO: 113 - protein, 213 aa, synthetic construct; identical to SEQ ID NO: 99

SEQ ID NO: 114 - protein, 44 aa, Rattus norvegicus
WADPNSTTEEAATQKEKWFENEWQGKNPPTPSEDSHVTEGTTAS

SEQ ID NO: 115 - protein, 43 aa, Homo sapiens
QATPSSTTEETATQKEQWFGNRWHEGYRQTPREDSHSTTGTAA

SEQ ID NO: 116 - protein, 107 aa, synthetic construct [sequence omitted]

SEQ ID NO: 117 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 118 - protein, 15 aa, synthetic construct
EPKSCDKTHTCPPCP

SEQ ID NO: 119 - protein, 110 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 120 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 10 aa, synthetic construct
GFSFGSYAMS

SEQ ID NO: 122 - protein, 10 aa, synthetic construct
GFTFSSYAMS

SEQ ID NO: 123 - protein, 10 aa, synthetic construct
GFSFSSYAMS

SEQ ID NO: 124 - protein, 10 aa, synthetic construct
GFSFSSFAMS

SEQ ID NO: 125 - protein, 10 aa, synthetic construct
GFSFGSFAMS

SEQ ID NO: 126 - protein, 10 aa, synthetic construct
GFSFRSFAMS

SEQ ID NO: 127 - protein, 10 aa, synthetic construct
GFTFRSFAMS

SEQ ID NO: 128 - protein, 10 aa, synthetic construct
GFSFRNFAMS

SEQ ID NO: 129 - protein, 10 aa, synthetic construct
AISAGGSSTF

SEQ ID NO: 130 - protein, 10 aa, synthetic construct
AISGGGSSTF

SEQ ID NO: 131 - protein, 10 aa, synthetic construct
AISGGGSSTY

SEQ ID NO: 132 - protein, 10 aa, synthetic construct
AISAGGSSTY

SEQ ID NO: 133 - protein, 10 aa, synthetic construct
AISAGGTSTY

SEQ ID NO: 134 - protein, 10 aa, synthetic construct
AISAGGYSTY

SEQ ID NO: 135 - protein, 10 aa, synthetic construct
TISAGGSSTF

SEQ ID NO: 136 - protein, 10 aa, synthetic construct
AISGGGYSTY

SEQ ID NO: 137 - protein, 10 aa, synthetic construct
AISGGGGSTF

SEQ ID NO: 138 - protein, 10 aa, synthetic construct
AISGSGRSTF

SEQ ID NO: 139 - protein, 4 aa, synthetic construct
TASS

SEQ ID NO: 140 - protein, 4 aa, synthetic construct
SASS

SEQ ID NO: 141 - protein, 4 aa, synthetic construct
AASS

SEQ ID NO: 142 - protein, 4 aa, synthetic construct
TAST

SEQ ID NO: 143 - protein, 4 aa, synthetic construct
SASN

SEQ ID NO: 144 - protein, 4 aa, synthetic construct
AAST

SEQ ID NO: 145 - protein, 4 aa, synthetic construct
SASI

SEQ ID NO: 146 - protein, 4 aa, synthetic construct
TASN

SEQ ID NO: 147 - protein, 125 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHYYSDSDYRSSAAMDYWGQGTLVTVSS

SEQ ID NO: 148 - protein, 106 aa, synthetic construct; identical to SEQ ID NO: 57

SEQ ID NO: 149 - protein, 455 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHYYSDSDYRSSAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 213 aa, synthetic construct; identical to SEQ ID NO: 96